(19) United States
(12) Patent Application Publication
(10) Pub. No.: US 2024/0003878 A1
(43) Pub. Date: Jan. 4, 2024

(54) ENRICHED BIOACTIVE RENAL CELL POPULATIONS, CHARACTERISTICS AND USES THEREOF

(71) Applicants: Timothy A. BERTRAM, Grand Cayman (KY); Deepak JAIN, Winston-Salem, NC (US)

(72) Inventors: Timothy A. BERTRAM, Grand Cayman (KY); Deepak JAIN, Winston-Salem, NC (US)

(21) Appl. No.: 18/252,619

(22) PCT Filed: Nov. 12, 2021
(86) PCT No.: PCT/US21/59215

§ 371 Date: May 11, 2023

Related U.S. Application Data
(60) Provisional application No. 63/113,437, filed on Nov. 13, 2020.
(60) Provisional application No. 63/214,483, filed on Jun. 24, 2021.
(60) Provisional application No. 63/255,704, filed on Oct. 14, 2021.

(51) Int. Cl. G01N 33/569; C12Q 1/6876; A61K 35/22; A61K 31/728; A61P 13/12

(52) U.S. Cl. CPC G01N 33/56966; C12Q 1/6876; A61K 35/22; A61K 31/728; A61P 13/12; C12Q 2600/158

(57) ABSTRACT

Methods of identifying an enriched heterogeneous renal cell population having therapeutic potential, enriched heterogeneous renal cell populations having therapeutic potential and uses for same.

### BACKGROUND

Chronic kidney disease (CKD) is characterized by progressive nephropathy that, without therapeutic intervention, will worsen; ultimately the patient may reach end stage renal disease (ESRD). Prevalence data from the U.S. to Europe show that approximately 10% of the general population have stage 1-3 CKD (ERA, 2009; USRDS, 2011; Jha et al. Chronic kidney disease: global dimension and perspectives. Lancet. 2013; 382:260-72). Worldwide, the incidence and prevalence of CKD and ESRD are increasing while therapeutic outcomes remain poor (Shaw et al. Global estimates of the prevalence of diabetes for 2010 and 2030. Diabetes Res Clin Pract. 2010; 87:4-14). The prevalence of chronic kidney disease has increased>33% between 1996 and 2006 in the US alone (U.S. Renal Data System. Costs of CKD and ESRD. Minneapolis, M N, 2007). The growing incidence of CKD presents a significant public health threat whose impact is only predicted to grow.

The greatest cause of ESRD is diabetes mellitus (Postma and de Zeeuw, 2009), and the incidence of CKD continues to increase, primarily due to the increases in the incidence of type 2 diabetes (Postma and de Zeeuw, 2009). CKD is often accompanied by adverse outcomes owing to underlying comorbidities and/or risk factors including hypertension and renovascular disease (Khan et al., 2002; Stenvinkel P. Chronic kidney disease—a public health priority and harbinger of premature cardiovascular disease J Intern med. 2010; 268:456-67). Due to serious comorbidities, patients with CKD are 5-11 times more likely to suffer premature death than survive to progress to ESRD (Collins et al., 2003; Smith et al., 2004). In order to survive, ESRD patients require renal replacement therapy (dialysis or transplantation). Currently, >500,000 people in the United States require dialysis or a kidney transplant, accounting for >$22 billion annually in Medicare costs (6% of the total Medicare budget) (Annual Report of the U.S. Organ Procurement and Transplantation Network and the Scientific Registry of Transplant Recipients: Transplant Data 1998-2007. Rockville, MD: HHS/HRSA/HSB/DOT, 2008). Kidney transplantation is the definitive standard of care for CKD, providing better long-term survival (and cost effectiveness) than dialysis; however, there remains a chronic shortage of organs. Despite increases in both cadaveric and living kidney donors, the rate of transplantation per 100 dialysis patient-years in the United States is actually decreasing. Preventing or delaying adverse outcomes of CKD by intervening early in the disease is the primary strategy in CKD management. Unfortunately, early therapeutic approaches to prevent disease progression have not been successful.

New treatment paradigms involving tissue engineering and cellular-based applications can provide substantial and durable augmentation of kidney functions, slow progression of disease and improve quality of life in this patient population. These next-generation regenerative medicine technologies provide isolated renal cells as a therapeutic option for CKD (Presnell et al. WO/2010/056328 and Ilagan et al. PCT/US2011/036347). Injection of these bioactive renal cells into the kidneys of animal models for CKD has resulted in significant improvement in animal survival and kidney function.

There is a need in the art to identify cells having therapeutic potential for treatment of kidney disease, based on their regeneration or nephrogenic capacity, and to insure renal cell-based therapeutics meet an expected level of efficacy.

### BRIEF SUMMARY

The present disclosure describes a method of identifying an enriched heterogeneous renal cell population as having a therapeutic potential. In the method, it is determined whether cells of the enriched heterogeneous renal cell population express at least one nephrogenic marker. The enriched heterogeneous renal cell population is identified as having a therapeutic potential if cells of the enriched heterogeneous renal cell population express the at least one nephrogenic marker. The at least one nephrogenic marker includes one or more of SIX2, OSR1, LHX1, RET and FGF8.

The present disclosure also describes a further method of identifying an enriched heterogeneous renal cell population as having a therapeutic potential. In this method, expression level of one or more of genes RHAMM, C2, C3, C4, fibrinogen, coagulation factor XIII, TEK, KDR, Notch1, Notch3, Timp3, Vwf, Adam15, Gas6, Igfbp1 and Tm4sf4 by cells of the enriched heterogeneous renal cell population is determined. The enriched heterogeneous renal cell population is identified as having therapeutic potential if the expression level of the one or more genes by cells of the enriched heterogeneous renal cell population is increased relative to expression level of the one or more genes by cells of a control renal cell population.

The present disclosure describes yet another method of identifying an enriched heterogeneous renal cell population as having a therapeutic potential. In the method, it is determined whether cells of the enriched heterogeneous renal cell population express SIX2, OSR1, RET and podocin. The enriched heterogeneous renal cell population is identified as having therapeutic potential if cells of the enriched heterogeneous renal cell population are determined to express SIX2, OSR1, RET and podocin.

The present disclosure describes yet a further method of identifying an enriched heterogeneous renal cell population as having a therapeutic potential. In the method, it is determined whether cells of the enriched heterogeneous renal cell population express nephrin, podocin and LHX1. The enriched heterogeneous renal cell population is identified as having therapeutic potential if cells of the enriched heterogeneous renal cell population are determined to express nephrin, podocin and LHX1.

### BRIEF DESCRIPTION OF THE DRAWINGS

FIG. 1A provides a table describing cell markers detected as expressed by cells of selected renal cell populations, e.g., enriched heterogeneous renal cell populations, cell sources for those markers and structures that cells expressing those markers may be involved in developing in a kidney.

FIG. 1B provides a graph showing percent cells of selected renal cell populations, e.g., enriched heterogeneous renal cell populations, that were determined to the express the markers described in FIG. 1A by fluorescence activated cell sorting (FACS). Mean percent and 95% CI displayed.

FIG. 2 provides a scatter plot of Kyoto Encyclopedia of Genes and Genomes (KEGG) pathways enriched in differentially express gene (DEG) set. Vertical coordinates represent pathway name, and horizontal coordinates represent Rich factor. The size and color of each point represent the number of differential genes in the pathway and the range of different Q value, respectively. The dark points for TGF-beta signaling pathway, Salmonella infection, Rheumatoid arthritis, Pyrimidine metabolism, Proteoglycans in cancer, Pathways in cancer, Other types of O-glycan biosynthesis, Legionelliosis, Hippo signaling pathway, HTLV-1 infection, FoxO signaling pathway, Drug metabolism—other enzymes, and Cell cycle are all in the high, close to 1.00, Q value range. The points for AGE-RAGE signaling pathway in diabetic complications and Amoebiasis are all in the low, close to 0.00 Q value range. The points for Protein digestion and absorption, Small cell lung cancer, ECM-receptor interaction, Axon guidance and Arginine and proline metabolism are in the mid-low Q value range.

FIG. 3 provides an annotation graph for KEGG pathway of DEG set.

FIG. 4 shows a Violin_boxplot of differentially methylated regions (DMR) average methylation level distribution. Hyper-dmr refers to the dmr that is hypermethylated in the sample, and hypo-dmr refers to the dmr that is hypomethylated in the sample.

FIG. 5A-H provide scatter diagrams from FACS analysis of cells for expression of: (A) SIX2, (B) OSR1, (C) LHX1, (D) RET, (E) nephrin, (F) podocin, (G) FGF8 and (H) RACK-1. Top scatter diagram for each of (A)-(H), isotype control antibodies from which negative cells were gated were used. Bottom scatter diagram each of (A)-(H), antigen-specific antibodies were used to detect positive cells, gated away from the negative cells. Positive cell populations are either to the right of the vertical axis or above the horizontal axis.

### DETAILED DESCRIPTION

The kidney is a complex organ, made up of numerous different cell types including podocytes, mesangial cells, endothelial cells, fibroblasts, epithelial cells and numerous stem and progenitor cell populations that are organized across the renal parenchyma into discrete, specialized functional units or nephrons, which serve to selectively filter electrolytes from the vasculature. This complexity of the kidney makes it exceptionally difficult to generate solid kidney organ replacement structures.

The complicated process of mammalian kidney development begins in a region of mesoderm known as the intermediate mesoderm. The pro-nephros, or “first kidney” represents the initial step of lineage specification in kidney development arising from the intermediate mesoderm. The pro-nephros is a small, hollow ball of epithelial tubular cells connected to the pro-nephric duct. The pro-nephric duct then extends caudally while the pro-nephros itself degenerates. The intermediate mesoderm now forms the second kidney, or meso-nephros, otherwise known as the meso-nephric duct. In females this regresses, but in males, it eventually becomes the epididymis, or the connective tissue between the testis and the bladder. The meso-nephric kidney consists of about 30 tubules. The lateral tip of the meso-nephric tubule fuses with the meso-nephric duct, opening a passage from the excretory units to the cloaca. The cloaca eventually becomes the bladder and the rectum. Finally, the last kidney or meta-nephric mesenchyme develops from the ureteric bud, which sprouts out and branches extensively from the nephric duct, with each new growing tip acquiring a cap-like aggregate of meta-nephric blastema tissue, thereby giving the meta-nephros a lobulated appearance.

Bidirectional signaling between the ureteric bud and the meta-nephric mesenchyme is ultimately responsible for mediating the seminal events of nephrogenesis. The ureteric bud, an outgrowth of the nephric duct at E10.5, signals to the surrounding meta-nephric mesenchyme, inducing condensation of meta-nephric mesenchymal cells around the tips of the invading ureteric bud. These mesenchymal condensates then undergo a mesenchymal-epithelial transition, to form primitive epithelial vesicles known as renal vesicles. Continued branching of the ureteric bud leads to development of components of the collecting duct system and renal pelvis. Meanwhile, the renal vesicles undergo a systematic series of morphological changes, eventually fusing with the ureteric bud epithelia to form a continuous epithelial tubule, the S-shaped body. Infiltration of the S-shaped body by endothelial cells leads to formation of the glomerular vasculature. Continued branching morphogenesis from the ureteric bud epithelia in response to signaling from the neighboring meta-nephric mesenchyme in turn leads to induction of new aggregates of meta-nephric mesenchyme at ureteric bud tips and continued nephrogenic events. This iterative process of ureteric bud branching morphogenesis and induction of additional mesenchymal condensates continues along the radial axis of the developing kidney with the youngest nephrons induced towards the periphery.

The molecular genetics underlying branching morphogenesis in the developing kidney and concomitant nephrogenesis are complex. Briefly, induction of the ureteric bud is triggered through up-regulation of the secreted growth factor GDNF through its receptor RET. Expression of RET along the pro-nephric duct is highest at the site of ureteric bud formation. Knockout mutations of either GDNF or RET are embryonic lethal, and are associated with failure of ureteric budding and consequent abrogation of kidney and ureter formation. Up-regulation of GDNF expression is through the action of transcription factors including PAX2, SIX1, 2, 4.

Mesenchymal-epithelial transition during conversion of meta-nephric mesenchymal condensates to renal vesicles is controlled principally by proteins of the WNT family, including WNT9b and WNT4. To this end, knockouts of WNT4 die within 24 hours of birth; kidneys are small and abnormal and consist of undifferentiated meta-nephric mesenchyme. Other growth factors modulating aspects of ureteric bud branching and nephrogenesis include TGF-β, FGF2, FGF7, LIF and LIM1. Multiple interacting signaling pathways are additionally involved in regulating aspects of both ureteric bud branching and nephrogenesis. These include the canonical WNT/β-catenin pathway, the sonic hedgehog pathway and the BMP and FGF members of the TGF-β super-family signaling pathway.

Regionalization of the intermediate mesoderm along the anterior/posterior axis is marked by expression of certain key transcription factors, including PAX2, PAX8, OSR1 and WT1. OSR1, which initially specifies intermediate mesoderm from paraxial and lateral plate fates, is nonetheless critical for cap mesenchyme specification and survival. OSR1 expression becomes restricted to the cap mesenchyme and OSR1 null mice fail to show expression of key cap mesenchyme genes (PAX2, SIX2, GDNF, EYA1, and SALL1). In contrast, OSR1 is not required for the formation of the FOXD1+ stromal compartment despite being expressed in metanephric mesenchyme prior to the separation of these lineages.

The WNT9b gene is expressed in the epithelial Wolffian duct prior to induction of metanephros development. WNT9b expression continues in the ureteric bud where it is more pronounced in the stalk region than in the tips. The expression of WNT9b in the mice is maintained in the collecting ducts until adulthood. WNT9b-mediated induction in the cap mesenchyme initiates expression of WNT4, fibroblast growth factor 8 (FGF8), paired box 8 (PAX8), and LIM homeobox protein 1 (LHX1)-encoding genes. These genes fail to become expressed in the cap mesenchyme of WNT9b-deficient embryonic kidneys and no nephrons form, as a result, WNT9b knockout mice die soon after birth.

Observed expression of multiple markers typically associated with the earliest signaling events during embryonic nephrogenesis may indicate that an enriched heterogeneous renal cell population, expanded following isolation from a kidney and subjected to a separation step, may have de-differentiated and acquired more renal progenitor-like properties. Therefore, the introduction of the enriched heterogeneous renal cell population having these renal progenitor-like properties into diseased renal parenchyma may trigger onset of critical signaling cascades that normally mediate nephrogenesis, but in the context of the adult parenchyma, are interpreted as regeneration.

Described herein are a method of identifying an enriched heterogeneous renal cell population as having a therapeutic potential, a heterogeneous renal cell population identified as having therapeutic potential, and methods and uses of heterogeneous renal cell populations having therapeutic potential.

In the methods of identifying an enriched heterogeneous renal cell population as having a therapeutic potential, the therapeutic potential of the enriched heterogeneous renal cell population may be in the treatment a kidney disease, a tubular transport deficiency, or a glomerular filtration deficiency.

If the enriched heterogeneous renal cell population is identified as having the potential to treat a kidney disease, the kidney disease may be associated with any stage or degree of acute or chronic renal failure that results in a loss of the kidney's ability to perform the function of blood filtration and elimination of excess fluid, electrolytes and wastes from the blood. The kidney disease may include endocrine dysfunctions such as anemia, e.g., erythropoietin-deficiency, and mineral imbalance, e.g., Vitamin D deficiency. The kidney disease may originate in the kidney or it may be secondary to another condition, e.g., heart failure, hypertension, diabetes, autoimmune disease or liver disease. Alternatively, the kidney disease may develop after an acute injury to the kidney, or it may be the result of an anomaly of the kidney and/or urinary tract.

If the enriched heterogeneous renal cell population is identified as having a therapeutic potential, the enriched heterogeneous renal cell population may restore kidney function, stabilize kidney function, improve kidney function, reduce renal fibrosis, reduce renal inflammation, induce tubulogenesis in a kidney, induce nephrogenesis in kidney, induce glomerulogenesis in a kidney, or have a regenerative effect in a kidney of a patient in need of such treatment. If the enriched heterogeneous renal cell population is identified as having a therapeutic potential, the enriched heterogeneous renal cell population may restore mineral balance or alleviate anemia in a patient in need of such treatment. If the enriched heterogeneous renal cell population is identified as having a therapeutic potential, it may delay or prevent the need for dialysis, or it may delay or prevent the need for a kidney transplant in a patient in need of a treatment for a kidney disease.

In the methods of identifying an enriched heterogeneous renal cell population as having a therapeutic potential, it may be determined whether cells of the enriched heterogeneous renal cell population express at least one nephrogenic marker. The at least one nephrogenic marker, whose expression is determined in the methods, may be any one of SIX Homeobox 2 (SIX2), odd-skipped-related 1 (OSR1), LIM homeobox 1 (LHX1), rearranged during transfection (RET) or fibroblast growth factor 8 (FGF8). The at least one nephrogenic marker whose expression is determined in the method may be, or may include, any one, any two, any three, any four or all of SIX2, OSR1, LHX1, RET and FGF8.

In these methods of identifying an enriched heterogeneous renal cell population as having a therapeutic potential, the determination of expression of the at least one nephrogenic marker may be, or may include, a determining of expression of any two of nephrogenic markers SIX2, OSR1, LHX1, RET or FGF8. If the determination of expression is of any two of these nephrogenic markers, the two nephrogenic markers may be or may include SIX2 and OSR1, or SIX2 and LHX1, or SIX2 and RET, or SIX2 and FGF8, or OSR1 and LHX1, or OSR1 and RET, or OSR1 and FGF8, or LHX1 and RET, or LHX1 and FGF8 or RET and FGF8.

In the methods of identifying an enriched heterogeneous renal cell population as having a therapeutic potential, the determination of expression of the at least one nephrogenic marker may be, or may include, a determination of expression of any three of nephrogenic markers SIX2, OSR1, LHX1, RET or FGF8. If the determination of expression is of any three of these nephrogenic markers, the three nephrogenic markers may be or may include SIX2, OSR1 and LHX1, or SIX2, OSR1 and RET, or SIX2, OSR1 and FGF8, or SIX2, LHX1 and RET, or SIX2, LHX1 and FGF8, or SIX2, RET and FGF8, or OSR1, LHX1 and RET, or OSR1, LHX1 and FGF8, or OSR1, RET and FGF8, or LHX1, RET and FGF8.

In the methods of identifying an enriched heterogeneous renal cell population as having a therapeutic potential, the determination of expression of the at least one nephrogenic marker may be, or may include, a determination of expression of any four of nephrogenic markers SIX2, OSR1, LHX1, RET or FGF8. If the determination of expression is of any four of these nephrogenic markers, the four nephrogenic markers may be or may include any of SIX2, OSR1, LHX1 and RET, or SIX2, OSR1, LHX1 and FGF8, or SIX2, LHX1, RET and FGF8, or SIX2, OSR1, RET and FGF8 or OSR1. LHX1, RET and FGF8.

In the methods of identifying an enriched heterogeneous renal cell population as having a therapeutic potential, the determination of expression of the at least one nephrogenic marker may be, or may include, a determination of expression of each of nephrogenic markers SIX2, OSR1, LHX1, RET and FGF8.

In the methods of identifying an enriched heterogeneous renal cell population as having therapeutic potential, determining that cells of the heterogeneous enriched renal cell population express the at least one (e.g., the any one, or any two, or any three, or any four or all five) nephrogenic marker may identify the enriched heterogeneous renal cell population as having therapeutic potential.

In the methods of identifying an enriched heterogeneous renal cell population as having a therapeutic potential, the determining expression of the at least one nephrogenic marker may further include a determining percentage of cells of the enriched heterogeneous renal cell population that express the at least one nephrogenic marker. If the percentage of cells of the enriched heterogeneous renal cell population that express the at least one nephrogenic marker is determined, then the percentage of cells that express the any one, any two, any three, any four or all five of nephrogenic markers SIX2, OSR1, LHX1, RET and FGF8 may be determined. If the percentage of cells of the enriched heterogeneous renal cell population that express the at least one nephrogenic marker is determined, then the enriched heterogeneous renal cell population may be identified as having therapeutic potential if about a certain, or particular, percentage of cells of the enriched heterogeneous renal cell population express the any one, any two, any three, any four or all five of nephrogenic markers SIX2, OSR1, LHX1, RET and FGF8.

If the percentage of cells of the enriched heterogeneous renal cell population that express SIX2 is determined in the methods, and it is determined that at least about 0.02% of cells of the enriched heterogeneous renal cell population express SIX2, then the enriched heterogeneous renal cell population may be identified as having a therapeutic potential. Alternatively, if the percentage of cells of the enriched heterogeneous renal cell population that express SIX2 is determined to be at least about 0.04%, or at least about 0.1%, or at least about 0.5%, or at least about 1.0%, or at least about 1.5%, or at least about 2.0%, or at least about 2.5%, or at least about 3.0%, or at least about 3.5%, or at least about 4.0%, or at least about 4.5%, or at least about 5.0%, or at least about 5.5%, then the enriched heterogeneous renal cell population may be identified as having a therapeutic potential. If the percentage of cells of the enriched heterogeneous renal cell population that express SIX2 is determined to be greater than 0% and up to at most about 15.0%, to be greater than 0% and up to at most about 10.0%, or greater than 0% and up to at most about 9.5%, or greater than 0% and up to at most about 9.0%, or greater than 0% and up to at most about 8.5%, or greater than 0% and up to at most about 8.0%, or greater than 0% and up to at most about 7.5%, or greater than 0% and up to at most about 7.0%, or greater than 0% and up to at most about 6.5%, or greater than 0% and up to up to at most about 6.0%, then the enriched heterogeneous renal cell population may be identified as having a therapeutic potential. Further, if the percentage of cells of the enriched heterogeneous renal cell population that express SIX2 is determined to be between about 0.02% to about 15.0%, or between about 0.02% to about 10.0%, or between about 0.02% to about 9.0%, or between about 0.02% and about 8.0%, or between about 0.02% and about 7.0%, or between about 0.02% and about 6.0%, or between about 0.04% to about 15.0%, or between about 0.04% to about 10.0%, or between about 0.04% to about 9.0%, or between about 0.04% and about 8.0%, or between about 0.04% and about 7.0%, or between about 0.04% and about 6.0%, or between about 1.0% to about 15.0%, or between about 1.0% to about 10.0%, or between about 1.0% to about 9.0%, or between about 1.0% and about 8.0%, or between about 1.0% and about 7.0% or between about 1.0% and about 6.0%, then the enriched heterogeneous renal cell population may be identified as having a therapeutic potential.

If the percentage of cells of the enriched heterogeneous renal cell population that express OSR1 is determined in the methods, and it is determined that at least about 30% of cells of the enriched heterogeneous renal cell population express OSR1, then the enriched heterogeneous renal cell population may be identified as having a therapeutic potential. Alternatively, if the percentage of cells of the enriched heterogeneous renal cell population that express OSR1 is determined to be at least about 35%, or at least about 36%, or at least about 37%, or at least about 38%, or at least about 39%, or at least about 40%, or at least about 41%, or at least about 42%, or at least about 43%, or at least about 44%, or at least about 45% or at least about 50%, then the enriched heterogeneous renal cell population may be identified as having a therapeutic potential. If the percentage of cells of the enriched heterogeneous renal cell population that express OSR1 is determined to be greater than 0% and up to at most about 90%, or greater than 0% and up to at most about 88%, or greater than 0% and up to at most about 86%, or greater than 0% and up to at most about 84%, or greater than 0% and up to at most about 82%, or greater than 0% and up to at most about 80%, or greater than 0% and up to at most about 75% or greater than 0% and up to at most about 70%, then the enriched heterogeneous renal cell population may be identified as having a regenerative potential. Further, if the percentage of cells of the enriched heterogeneous renal cell population that express OSR1 is determined to be between about 30% and about 90%, or between about 30% and about 88%, or between about 30% and about 86%, or between about 30% and about 84%, or between about 30% and about 82%, or between about 30% and about 80%, or between 34% and 90%, or between about 34% and about 88%, or between about 34% and about 86%, or between about 34% and about 84%, or between about 34% and about 82%, or between about 34% and about 80%, or between about 36% and about 90%, or between about 36% and about 88%, or between about 36% and about 86%, or between about 36% and about 84%, or between about 36% and about 82% or between about 36% and about 80%, or between about 40% and about 90%, or between about 40% and about 85%, or between about 45% and about 90%, or between about 45% and about 85%, or between about 50% and about 90%, or between about 50% and about 85%, or between about 55% and about 90%, or between about 55% and about 85%, or between about 60% and about 90%, or between about 60% and about 85%, or between about 65% and about 90%, or between about 65% and about 85%, or between about 70% and about 90%, or between about 70% and about 85%, then the enriched heterogeneous renal cell population may be identified as having a regenerative potential.

If the percentage of cells of the enriched heterogeneous renal cell population that express LHX1 is determined in the methods, and it is determined that at least about 5% of cells of the enriched heterogeneous renal cell population express LHX1, the heterogeneous enriched renal cell population may be identified as having a regenerative potential. Alternatively, if the percentage of cells of the enriched heterogeneous renal cell population that express LHX1 is determined to be at least about 6%, or at least about 7%, or at least about 8%, or at least about 9% or at least about 10, then the heterogeneous renal cell population may be identified as having therapeutic potential. If the percentage of cells of the enriched heterogeneous renal cell population that express LHX1 is determined to be greater than 0% and up to at most 75%, or greater than 0% and up to at most 70%, or greater than 0% and up to at most about 65%, or greater than 0% and up to at most about 64%, or greater than 0% and up to at most about 63%, or greater than 0% and up to at most about 62%, or greater than 0% and up to at most about 61%, or greater than 0% and up to at most about 60%, or greater than 0% and up to at most about 59%, or greater than 0% and up to at most about 58%, or greater than 0% and up to at most about 57%, or greater than 0% and up to at most about 56% or greater than 0% and up to at most about 55%, then the heterogeneous renal cell population may be identified as having therapeutic potential. Furthermore, if the percentage of cells of the enriched heterogeneous renal cell population that express LHX1 is determined to be between about 6% and about 60%, or between about 6% and about 59%, or between about 6% and about 58%, or between about 6% and about 57%, or between about 6% and about 56%, or between about 6% and about 55%, or between about 6% and about 54%, or between about 8% and about 60%, or between about 8% and about 59%, or between about 8% and about 58%, or between about 8% and about 57%, or between about 8% and about 56%, or between about 8% and about 55%, or between about 8% and about 54%, or between about 10% and about 60%, or between about 10% and about 59%, or between about 10% and about 58%, or between about 10% and about 57%, or between about 10% and about 56%, or between about 10% and about 55% or between about 10% and about 54%, or between about 16% and 80%, or between about 16% and 70%, or between about 16% and about 60%, or between about 16% and about 58%, or between about 16% and about 56%, or between about 16% and about 54%, or between about 16% and about 52%, or between about 16% and about 50%, or between 22% and about 60%, or between about 22% and about 58%, or between about 22% and about 56%, or between about 22% and about 54%, or between about 22% and about 52%, or between about 22% and about 50%, then the enriched heterogeneous renal cell population may be identified as having a regenerative potential.

If the percentage of cells of the enriched heterogeneous renal cell population that express RET is determined in the methods, and it is determined that at least about 45% of cells of the enriched heterogeneous renal cell population express RET, then the enriched heterogeneous renal cell population may be identified as having therapeutic potential. Alternatively, if the percentage of cells of the enriched heterogeneous renal cell population that express RET is determined to be at least about 22%, at least about 24%, at least about 26%, at least about 28%, at least about 30%, at least about 32%, at least about 34%, at least about 36%, at least about 38%, at least about 40%, at least about 42%, at least about 44%, at least about 46%, or at least about 47%, or at least about 48%, or at least about 49%, or at least about 50%, or at least about 51%, or at least about 52%, or at least about 53% or at least about 54%, then the enriched heterogeneous renal cell population may be identified as having a therapeutic potential. If the percentage of cells of the enriched heterogeneous renal cell population that express RET is determined to be greater than 0% and up to at most about 95%, or greater than 0% and up to at most about 94%, or greater than 0% and up to at most about 93%, or greater than 0% and up to at most about 92%, or greater than 0% and up to at most about 91%, or greater than 0% and up to at most about 90%, or greater than 0% and up to at most about 89%, or greater than 0% and up to at most about 88%, or greater than 0% and up to at most about 87%, or greater than 0% and up to at most about 86% or greater than 0% and up to at most about 85%, then the enriched heterogeneous renal cell population may be identified as having a therapeutic potential. Furthermore, if the percentage of cells of the enriched heterogeneous renal cell population that express RET is determined to be between about 45% and about 95%, or between about 45% and about 94%, or between about 45% and about 93%, or between about 45% and about 92%, or between about 45% and about 91%, or between about 45% and about 90%, or between about 45% and about 89%, or between about 45% and about 88%, or between about 47% and about 95%, or between about 47% and about 94%, or between about 47% and about 93%, or between about 47% and about 92%, or between about 47% and about 91%, or between about 47% and about 90%, or between about 47% and about 89%, or between about 47% and about 88%, or between about 49% and about 95%, or between about 49% and about 94%, or between about 49% and about 93%, or between about 49% and about 92%, or between about 49% and about 91%, or between about 49% and about 90%, or between about 49% and about 89% or between about 49% and about 88%, or between about 20% to about 60%, or between about 20% to about 55%, or between about 20% to about 50%, or between about 20% to about 45%, or between about 20% to about 40%, or between about 25% to about 60%, or between about 25% to about 55%, or between about 25% to about 50%, or between about 25% to about 45%, or between about 25% to about 40%, then the enriched heterogeneous renal cell population may be identified as having a therapeutic potential.

If the percentage of cells of the enriched heterogeneous renal cell population that express FGF8 is determined in the methods, and it is determined that at least about 0.2% of cells of the enriched heterogeneous renal cell population express FGF8, then the enriched heterogeneous renal cell population may be identified as having therapeutic potential. Alternatively, if the percentage of cells of the enriched heterogeneous renal cell population that express FGF8 is determined to be at least about 0.25%, or at least about 0.3%, or at least about 0.35%, or at least about 0.4%, or at least about 0.45%, or at least about 0.48%, or at least about 0.5%, or at least about 0.55%, or at least about 0.6%, or at least about 0.8%, or at least about 1.0%, or at least about 1.5%, or at least about 2.0% or at about 2.5%, then the enriched heterogeneous renal cell population may be identified as a having a therapeutic potential. If the percentage of cells of the enriched heterogeneous renal cell population that express FGF8 is determined to be greater than 0% and up to at most about 65% or greater than 0% and up to at most about 64%, or greater than 0% and up to at most about 63%, or greater than 0% and up to at most about 62%, or greater than 0% and up to at most about 61%, or greater than 0% and up to at most about 60%, or greater than 0% and up to at most about 59%, or at most about 58%, or greater than 0% and up to at most about 57%, or greater than 0% and up to at most about 56% or greater than 0% and up to at most about 55%, then the enriched heterogeneous renal cell population may be identified as having a therapeutic potential. Furthermore, if the percentage of cells of the enriched heterogeneous renal cell population that express FGF8 is determined to be between about 0.3% and about 64%, or between about 0.3% and about 63%, or between about 0.3% and about 62%, or between about 0.3% and about 61%, or between about 0.3% and about 60%, or between about 0.3% and about 59%, or between about 0.3% and about 58%, or between about 0.3% and about 57%, or between about 0.4% and about 64%, or between about 0.4% and about 63%, or between about 0.4% and about 62%, or between about 0.4% and about 61%, or between about 0.4% and about 60%, or between about 0.4% and about 59%, or between about 0.4% and about 58%, or between about 0.4% and about 57%, or between about 0.5% and about 64%, or between about 0.5% and about 63%, or between about 0.5% and about 62%, or between about 0.5% and about 61%, or between about 0.5% and about 60%, or between about 0.5% and about 59%, or between about 0.5% and about 58% or between about 0.5% and about 57%, or between about 1% and about 64%, or between about 1% and about 54%, or between about 1% and about 44%, or between about 1% and about 34%, or between 1% and about 24%, or between about 2% and about 64%, or between about 2% and about 54%, or between about 2% and about 44%, or between about 2% and about 34%, or between 2% and about 24%, or between about 3% and about 64%, or between about 3% and about 54%, or between about 3% and about 44%, or between about 3% and about 34%, or between 3% and about 24% then the enriched heterogeneous renal cell population may be identified as having a therapeutic potential.

The method of identifying an enriched heterogeneous renal cell population as having a therapeutic potential may include a step of determining the percentage of cells of the heterogeneous renal cell population that express a combination of any two, any three, any four or all five of nephrogenic markers SIX2, OSR1, LHX1, RET and FGF8. For example, the enriched heterogeneous renal cell population may be identified as having a therapeutic potential if a combination of any two, any three, any four or all five of the following are determined: greater than 0% and up to at most about 6% of cells of the heterogeneous renal cell population express SIX2, at least about 36% of cells of the heterogeneous renal cell population express OSR1, at least about 8% of cells of the enriched heterogeneous renal cell population express LHX1, at least about 49% of cells of the enriched heterogeneous renal cell population express RET and/or greater than 0% and up to at most about 59% of cells of the enriched heterogeneous renal cell population to express FGF8. In another example, the enriched heterogeneous renal cell population may be identified as having a therapeutic potential if any two, any three, any four or all five of the following are determined: at least about 0.04% of cells of enriched heterogeneous renal cell population express SIX2, greater than 0% and up to at most about 85% of cells of the enriched heterogeneous renal cell population express OSR1, greater than 0% and up to at most about 58% of cells of the enriched heterogeneous renal cell population express LHX1, greater than 0% and up to at most about 90% of cells of the enriched heterogeneous renal cell population express RET and/or at least about 0.48% of cells of the enriched heterogeneous renal cell population express FGF8. An enriched heterogeneous renal cell population may further be identified as having a therapeutic potential if any two, any three, any four, or all five of the following are determined: between about 0.04% and about 6.0% of cells of the enriched heterogeneous renal cell population express SIX2, between about 36% and about 85% of cells of the enriched heterogeneous renal cell population express OSR1, between about 8% and about 58% of cells of the enriched heterogeneous renal cell population express LHX1, between about 49% and about 90% of cells of the enriched heterogeneous renal cell population express RET and between about 0.48% and/or about 59% of cells express FGF8. The combination of any two, any three, any four or all five nephrogenic markers whose expression may be determined at these percentages may be a combination of any of SIX2 and OSR1; or SIX2 and LHX1; or SIX2 and RET; or SIX2 and FGF8; or OSR1 and LHX1; or OSR1 and RET; or OSR1 and FGF8; or LHX1 and RET; or LHX1 and FGF8; or RET and FGF8; or SIX2, OSR1 and LHX1; or SIX2, OSR1 and RET; or SIX2, OSR1 and FGF8; or SIX2, LHX1 and RET; or SIX2, LHX1 and FGF8; or SIX2, RET and FGF8; or OSR1, LHX1 and RET; or OSR1, LHX1 and FGF8; or OSR1, RET and FGF8; or LHX1, RET and FGF8; or SIX2, OSR1, LHX1 and RET; SIX2, OSR1, LHX1 and FGF8; SIX2, LHX1, RET and FGF8; SIX2, OSR1, RET and FGF8; OSR1, LHX1, RET and FGF8; or SIX2, OSR1, LHX1, RET and FGF8.

In the method of identifying an enriched heterogeneous renal cell population as having a therapeutic potential, the enriched heterogeneous renal cell population may be alternatively be identified as having a therapeutic potential if any two, any three, any four or all five of the following are determined: greater than 0% and up to at most about 10% of cells of the heterogeneous renal cell population express SIX2, at least about 30% of cells of the heterogeneous renal cell population express OSR1, at least about 5% of cells of the enriched heterogeneous renal cell population express LHX1, at least about 40% of cells of the enriched heterogeneous renal cell population express RET and/or greater than 0% and up to at most about 60% of cells of the enriched heterogeneous renal cell population to express FGF8. In another example, the enriched heterogeneous renal cell population may be identified as having a therapeutic potential if any two, any three, any four or all five of the following are determined: at least about 0.02% of cells of the enriched heterogeneous renal cell population express SIX2, greater than 0% and up to at most about 90% of cells of the enriched heterogeneous renal cell population express OSR1, greater than 0% and up to at most about 65% of cells of the enriched heterogeneous renal cell population express LHX1, greater than 0% and up to at most about 95% of cells of the enriched heterogeneous renal cell population express RET and/or at least about 0.4% of cells of the enriched heterogeneous renal cell population express FGF8. Further, the enriched heterogeneous renal cell population may be identified as having a therapeutic potential if any two, any three, any four, or all five of the following are determined: between about 0.02% and about 10.0% of cells of the enriched heterogeneous renal cell population express SIX2, between about 30% and about 90% of cells of the enriched heterogeneous renal cell population express OSR1, between about 5% and about 65% of cells of the enriched heterogeneous renal cell population express LHX1, between about 40% and about 95% of cells of the enriched heterogeneous renal cell population express RET and/or between about 0.4% and about 60% of cells express FGF8. The combination of any two, any three, any four or all five nephrogenic markers whose expression may be determined at these percentages may be a combination of any of SIX2 and OSR1; or SIX2 and LHX1; or SIX2 and RET; or SIX2 and FGF8; or OSR1 and LHX1; or OSR1 and RET; or OSR1 and FGF8; or LHX1 and RET; or LHX1 and FGF8; or RET and FGF8; or SIX2, OSR1 and LHX1; or SIX2, OSR1 and RET; or SIX2, OSR1 and FGF8; or SIX2, LHX1 and RET; or SIX2, LHX1 and FGF8; or SIX2, RET and FGF8; or OSR1, LHX1 and RET; or OSR1, LHX1 and FGF8; or OSR1, RET and FGF8; or LHX1, RET and FGF8; or SIX2, OSR1, LHX1 and RET; SIX2, OSR1, LHX1 and FGF8; SIX2, LHX1, RET and FGF8; SIX2, OSR1, RET and FGF8; OSR1, LHX1, RET and FGF8; or SIX2, OSR1, LHX1, RET and FGF8.

In the method of identifying an enriched heterogeneous renal cell population as having a therapeutic potential, the enriched heterogeneous renal cell population may be identified as having a therapeutic potential if any two, any three, any four or all five of the following are determined: greater than 0% and up to at most about 3% of cells of the heterogeneous renal cell population express SIX2, at least about 60% of cells of the heterogeneous renal cell population express OSR1, at least about 25% of cells of the enriched heterogeneous renal cell population express LHX1, at least about 65% of cells of the enriched heterogeneous renal cell population express RET and/or greater than 0% and up to at most about 35% of cells of the enriched heterogeneous renal cell population to express FGF8. In another example, the enriched heterogeneous renal cell population may be identified as having a therapeutic potential if any two, any three, any four or all five of the following are determined: at least about 0.5% of cells of the enriched heterogeneous renal cell population express SIX2, greater than 0% and up to at most about 80% of cells of the enriched heterogeneous renal cell population express OSR1, greater than 0% and up to at most about 55% of cells of the enriched heterogeneous renal cell population express LHX1, greater than 0% and up to at most about 90% of cells of the enriched heterogeneous renal cell population express RET and/or at least about 5% of cells of the enriched heterogeneous renal cell population express FGF8. Further, the enriched heterogeneous renal cell population may be identified as having a therapeutic potential if any two, any three, any four, or all five of the following are determined: between about 0.5% and about 3.0% of cells of the enriched heterogeneous renal cell population express SIX2, between about 60% and about 80% of cells of the enriched heterogeneous renal cell population express OSR1, between about 25% and about 55% of cells of the enriched heterogeneous renal cell population express LHX1, between about 65% and about 90% of cells of the enriched heterogeneous renal cell population express RET and/or between about 5% and about 35% of cells express FGF8. The combination of any two, any three, any four or all five nephrogenic markers whose expression may be determined at these percentages may be a combination of any of SIX2 and OSR1; or SIX2 and LHX1; or SIX2 and RET; or SIX2 and FGF8; or OSR1 and LHX1; or OSR1 and RET; or OSR1 and FGF8; or LHX1 and RET; or LHX1 and FGF8; or RET and FGF8; or SIX2, OSR1 and LHX1; or SIX2, OSR1 and RET; or SIX2, OSR1 and FGF8; or SIX2, LHX1 and RET; or SIX2, LHX1 and FGF8; or SIX2, RET and FGF8; or OSR1, LHX1 and RET; or OSR1, LHX1 and FGF8; or OSR1, RET and FGF8; or LHX1, RET and FGF8; or SIX2, OSR1, LHX1 and RET; SIX2, OSR1, LHX1 and FGF8; SIX2, LHX1, RET and FGF8; SIX2, OSR1, RET and FGF8; OSR1, LHX1, RET and FGF8; or SIX2, OSR1, LHX1, RET and FGF8.

In the method of identifying an enriched heterogeneous renal cell population as having a therapeutic potential, the enriched heterogeneous renal cell population may be identified as having a therapeutic potential if any two, any three, any four or all five of the following are determined: greater than 0% and up to at most about 10% of cells of the heterogeneous renal cell population express SIX2, at least about 35% of cells of the heterogeneous renal cell population express OSR1, at least about 8% of cells of the enriched heterogeneous renal cell population express LHX1, at least about 20% of cells of the enriched heterogeneous renal cell population express RET and/or greater than 0% and up to at most about 60% of cells of the enriched heterogeneous renal cell population to express FGF8. In another example, the enriched heterogeneous renal cell population may be identified as having a therapeutic potential if any two, any three, any four or all five of the following are determined: at least about 0.2% of cells of the enriched heterogeneous renal cell population express SIX2, greater than 0% and up to at most about 85% of cells of the enriched heterogeneous renal cell population express OSR1, greater than 0% and up to at most about 65% of cells of the enriched heterogeneous renal cell population express LHX1, greater than 0% and up to at most about 90% of cells of the enriched heterogeneous renal cell population express RET and/or at least about 0.5% of cells of the enriched heterogeneous renal cell population express FGF8. Further, the enriched heterogeneous renal cell population may be identified as having a therapeutic potential if any two, any three, any four, or all five of the following are determined: between about 0.2% and about 10.0% of cells of the enriched heterogeneous renal cell population express SIX2, between about 35% and about 85% of cells of the enriched heterogeneous renal cell population express OSR1, between about 8% and about 65% of cells of the enriched heterogeneous renal cell population express LHX1, between about 20% and about 90% of cells of the enriched heterogeneous renal cell population express RET and/or between about 0.5% and about 60% of cells express FGF8. The combination of any two, any three, any four or all five nephrogenic markers whose expression may be determined at these percentages may be a combination of any of SIX2 and OSR1; or SIX2 and LHX1; or SIX2 and RET; or SIX2 and FGF8; or OSR1 and LHX1; or OSR1 and RET; or OSR1 and FGF8; or LHX1 and RET; or LHX1 and FGF8; or RET and FGF8; or SIX2, OSR1 and LHX1; or SIX2, OSR1 and RET; or SIX2, OSR1 and FGF8; or SIX2, LHX1 and RET; or SIX2, LHX1 and FGF8; or SIX2, RET and FGF8; or OSR1, LHX1 and RET; or OSR1, LHX1 and FGF8; or OSR1, RET and FGF8; or LHX1, RET and FGF8; or SIX2, OSR1, LHX1 and RET; SIX2, OSR1, LHX1 and FGF8; SIX2, LHX1, RET and FGF8; SIX2, OSR1, RET and FGF8; OSR1, LHX1, RET and FGF8; or SIX2, OSR1, LHX1, RET and FGF8.

It should be understood that if the percentage of cells expressing a certain marker is provided as being a percentage of “about” a particular number e.g., about 5%, the percentage of cells need not be exactly the particular number, e.g., exactly 5%. Rather, it should be understood that if the percentage of cells expressing a certain marker is provided as being “about” a particular number, e.g., about 5%, then the percentage of cells expressing the certain marker may be within up to 10% of that particular number, e.g., between 4.5% and 5.5%.

In the methods of identifying an enriched heterogeneous renal cell population as having a therapeutic potential, it may be determined whether cells of the enriched heterogeneous renal cell population express one or more further markers, i.e., markers other than nephrogenic markers SIX2, OSR1, LHX1, RET and FGF8. The one or more further markers may be, or may include, one or more of podocyte markers, epithelial markers, developmental markers, or miRNAs. If the one or more further markers include a podocyte marker, the one or more further markers may include one or more nephrin, EpiCAM, NPHS2 (encoding podocin), podocin, Wilms' tumor protein (WT1), or podocalyxin. If the one or more further markers include an epithelial cell marker, the one or more further markers may include one or more of E-cadherin, N-cadherin, cubulin/megalin, vitamin D-25 hydroxylase (CYP2R1), Gamma-glutamyltransferase 1 (GGT1), liver-enriched transcriptional protein (LAP), cytokeratin (CK) 18, Aquaporin (AQP) 2, Kidney injury molecule (KIM1), erythropoietin (EPO), Kinase Insert Domain Receptor (KDR), Epithelial cell adhesion molecule (ECAM), or AQP1. If the one or more further markers includes a developmental marker, the developmental marker may include one or more of neutrophil gelatinase-associated lipocalin (NGAL), sonic hedgehog (SHH), NOTCH, C-X-C Motif Chemokine Receptor 4 (CXCR4), lunatic fringe (LFNG) or IL-11. The one or more further markers may be or may include receptor for activated C kinase 1 (RACK-1). If the one or more further markers include an miRNA, the miRNA may be one or more of miR22, miR181 or miR145.

If the one or more further markers includes a podocyte marker, the podocyte marker may be nephrin. In such a method, if cells of the enriched heterogeneous renal cell population are further determined to express nephrin, the enriched heterogeneous renal cell population may be identified as having a therapeutic potential. If the one or more further markers is, or includes, nephrin, the percentage of cells of the enriched heterogeneous renal cell population that express nephrin may be determined. If the percentage of cells of the enriched heterogeneous renal cell population that express nephrin is determined, the enriched heterogeneous renal cell population may be identified as having a therapeutic potential if at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%, of cells of the heterogeneous renal cell population express nephrin. If the percentage of cells of the enriched heterogeneous renal cell population that express nephrin is determined, the enriched heterogeneous renal cell population may be identified as having a therapeutic potential if between about 4% and about 95%, or between about 10% and about 95%, or between about 15% and about 95%, or between about 20% and about 95%, or between about 25% and about 95%, and between about 30% and about 95%, or between 35% and about 95%, or between about 40% and about 95%, or between about 45% and 95%, or between about 50% and about 95%, or between about 55% and about 95%, or between about 60% and about 95%, or between about 65% and about 95%, or between 70% and about 95%, or between about 75% and about 95%, or between about 80% and about 95% or between about 85% and about 95% of cells of the heterogeneous renal cell population express nephrin.

If the one or more further markers is or includes a podocyte marker, the podocyte marker may be podocin. In such a method, if cells of the enriched heterogeneous renal cell population are further determined to express podocin, the enriched heterogeneous renal cell population may be identified as having a therapeutic potential. If the one or more further markers is, or includes, podocin, the percentage of cells of the enriched heterogeneous renal cell population that express podocin may be determined. If the percentage of cells of the enriched heterogeneous renal cell population that express podocin is determined, the enriched heterogeneous renal cell population may be identified as having a therapeutic potential if at least about 80%, at least about 82%, at least about 84%, at least about 86%, at least about 88%, at least about 90%, at least about 92%, at least about 94%, at least about 96% or at least about 98% of cells of the heterogeneous renal cell population express podocin.

If the one or more further markers includes a podocyte marker, the podocyte marker may include both nephrin and podocin. In such a method, if cells of the enriched heterogeneous renal cell population are further determined to express nephrin and podocin, the enriched heterogeneous renal cell population may be identified as having a therapeutic potential. If the one or more further markers include nephrin and podocin, the percentage of cells of the enriched heterogeneous renal cell population that express nephrin and podocin may be determined. If the percentage of cells of the enriched heterogeneous renal cell population that express nephrin and podocin is determined, the enriched heterogeneous renal cell population may be identified as having a therapeutic potential if at least 4% of cells express nephrin and at least about 90% of cells express podocin, or if at least 8% of cells express nephrin and at least about 90% of cell express podocin, at if at least 10% of cells express nephrin and at least about 90% of cell express podocin, or if at least 12% of cells express nephrin and at least about 90% of cell express podocin, or if at least 14% of cells express nephrin and at least about 90% of cell express podocin, or if at least 20% of cells express nephrin and at least about 90% of cell express podocin, or if at least 25% of cells express nephrin and at least about 90% of cell express podocin, or if at least 30% of cells express nephrin and at least about 90% of cell express podocin, or if at least 35% of cells express nephrin and at least about 90% of cell express podocin, or if at least 40% of cells express nephrin and at least about 90% of cell express podocin, or if at least 45% of cells express nephrin and at least about 90% of cell express podocin, or if at least 50% of cells express nephrin and at least about 90% of cell express podocin, or if at least 55% of cells express nephrin and at least about 90% of cell express podocin, or if at least 60% of cells express nephrin and at least about 90% of cell express podocin, or if at least 65% of cells express nephrin and at least about 90% of cell express podocin, or if at least 70% of cells express nephrin and at least about 90% of cell express podocin, or if at least 75% of cells express nephrin and at least about 90% of cell express podocin.

If the one or more further markers is, or includes, RACK-1, the enriched heterogeneous renal cell population may be identified as having a therapeutic potential if cells of the population express RACK-1. If the one or more further markers is, or includes, RACK-1, the percentage of cells of the enriched heterogeneous renal cell population that express RACK-1 may be determined. If the percentage of cells of the enriched heterogeneous renal cell population that express RACK-1 is determined, the enriched heterogeneous renal cell population may be identified as having a therapeutic potential if at least about 80%, at least about 82%, at least about 84%, at least about 86%, at least about 88%, at least about 90%, at least about 92%, at least about 94%, at least about 96%, or at least about 98% of cells of the heterogeneous renal cell population express RACK-1.

If the one or more further markers includes an epithelial cell marker, the epithelial cell marker may be CYP2R1. In such a method, if cells of the enriched heterogeneous renal cell population are further determined to express CYP2R1, the enriched heterogeneous renal cell population may be identified as having a therapeutic potential. If the one or more further markers is, or includes CYP2R1, the percentage of cells of the enriched heterogeneous renal cell population that express CYP2R1 may be determined. If the percentage of cells of the enriched heterogeneous renal cell population that express CYP2R1 is determined, the enriched heterogeneous renal cell population may be identified as having a therapeutic potential if between about 75% and about 100%, or between about 80% and about 100%, or between about 85% and about 100%, or between about 86% and about 100%, or between about 87% and about 100%, or between about 88% and about 100%, or between about 75% and about 97%, or between about 80% and about 97%, or between about 85% and about 97%, or between about 86% and about 97%, or between about 87% and about 97% or between about 88% and about 97% of cells of the heterogeneous renal cell population express CYP2R1.

If the one or more further markers includes a developmental marker, the developmental marker may be CXCR4. In such a method, if cells of the enriched heterogeneous renal cell population are further determined to express CXCR4, then the enriched heterogeneous renal cell population may be identified as having a therapeutic potential. If the one or more further markers is, or includes CXCR4, the percentage of cells of the enriched heterogeneous renal cell population that express CXCR4 may be determined. If the percentage of cells of the enriched heterogeneous renal cell population that express CXCR4 is determined, the enriched heterogeneous renal cell population may be identified as having a therapeutic potential if greater than about 15%, or greater than about 16%, or greater than about 17%, or greater than about 18%, or greater than about 1⁹%, or greater than about 20%, or greater than about 21%, or greater than about 22%, or greater than about 23%, or greater than about 24% or greater than about 25% of cells of the heterogeneous renal cell population express CXCR4.

The one or more further markers may include combinations of one or more of bone morphogenetic protein (BMP)4, BMP7, glial cell derived neurotrophic factor (GDNF), homeobox protein HOX11, eyes absent homolog 1 (EYA1), SAL1 and SIX4 (SIX Homeobox 4). The one or more further markers may include combinations of one or more of Paired Box 2 (PAX2), Cbp/P300 interacting transactivator with Glu/Asp rich carboxy-terminal domain 1 (CITED1), Fibroblast growth factor receptor (FGFR)1, FGF7, FGF10, homeobox protein HOX10, capsuling/edicardin/Tcf21 (POD1) POD1 and mucin 1 (MUC1). The one or more further markers may include combinations of one or more of Receptor for Hyaluronan Mediated Motility (RHAMM), Complement Component C2, Complement Component C3, Complement Component C4, fibrinogen, coagulation factor XIII, TEK tyrosine kinase, KDR, Notch1, Notch3, Timp3, von Willebrand Factor (VWF), Adam15, Growth arrest-specific 6 (Gas6), Insulin like growth factor binding protein (Igfbp) 1, or Transmembrane 4 Superfamily member 4 (Tm4sf4). The one or more further markers may include RHAMM.

The one or more further markers may include combinations of one or more of CDK2A, interleukin 11 (IL11), transcription growth factor (TGF)β2, fibronectin (FN)1, cysteine rich secretory protein LCCL domain containing 2 (CRISPLD2), collagen type 1 alpha 1 chain (COL1A1), lysyl oxidase (LOX), runt-related transcription factor 2 (RUNX2), lunatic fringe (LFNG), brain-derived neurotrophic factor (BDNF), claudin (CLDN)3, uridine phosphorylase (UPP)1, Kruppel-like factor (KLF)14, glycosyltransferase-like (GYLTL)1B, mannosidase alpha class 1C member 1 (MAN1C1), polypeptide N-acetylgalactosaminyltransferase 9 (GALNT9), aquaporin (AQP1), solute carrier family 47 member 1 (SLC47A1), WNK lysine deficient protein kinase (WNK)2, calcium-sensing receptor (CASR), retinoic acid induced 2 (RAI2), plasmalemma vesicle associated protein (PLVAP), shisa family member (SHISA)3, prostate androgen-regulated mucin-like protein 1 (PARM1), FGF11, forkhead box E1 (FOXE1), WNT family member (WNT)5A, WNT10A, TGFβ1 and insulin like growth factor binding protein (IGFBP)3. The one or more further markers may include any one or more of IL11, TGFβ2, CRISPLD2, LOX, LNFG, BDNF, WNT5A or IGFBP3.

In some methods of identifying an enriched heterogeneous renal cell population as having therapeutic potential, it may be determined whether cells of the enriched heterogeneous renal cell population express SIX2, OSR1, RET and podocin. In such methods, the enriched heterogeneous renal cell population may be identified as having therapeutic potential if cells of the enriched heterogeneous renal cell population are determined to express SIX2, OSR1, RET and podocin. The determining that cells of the enriched heterogeneous renal cell population express SIX2, OSR, RET and podocin may include a determining of a percentage of cells of the enriched heterogeneous renal cell population that express SIX2, OSR, RET and podocin.

If the determining that cells of the enriched heterogeneous renal cell population express SIX2, OSR, RET and podocin includes determining a percentage of cells of the enriched heterogeneous renal cell population that express SIX2, OSR, RET and podocin, then the percentage of cells of the population that express: (i) SIX2 may be greater than 0% and up to at most 15.0%, or greater than 0% and up to at most about 10.0%, or greater than 0% and up to at most about 9.5%, or greater than 0% and up to at most about 9.0%, or greater than 0% and up to at most about 8.5%, or greater than 0% and up to at most about 8.0%, or greater than 0% and up to at most about 7.5%, or greater than 0% and up to at most about 7.0%, or greater than 0% and up to at most about 6.5%, or greater than 0% and up to up to at most about 6.0%, or between about 0.02% to about 15%, or between about 0.02% to about 10.0%, or between about 0.02% to about 9.0%, or between about 0.02% and about 8.0%, or between about 0.02% and about 7.0%, or between about 0.02% and about 6.0%, or between about 0.04% to about 15%, or between about 0.04% to about 10.0%, or between about 0.04% to about 9.0%, or between about 0.04% and about 8.0%, or between about 0.04% and about 7.0%, or between about 0.04% and about 6.0%, or between about 1.0% and about 15.0%, or between about 1.0% to about 10.0%, or between about 1.0% to about 9.0%, or between about 1.0% and about 8.0%, or between about 1.0% and about 7.0% or between about 1.0% and about 6.0%; (ii) OSR may be greater than 0% and up to at most about 90%, or greater than 0% and up to at most about 88%, or greater than 0% and up to at most about 86%, or greater than 0% and up to at most about 84%, or greater than 0% and up to at most about 82%, or greater than 0% and up to at most about 80%, or greater than 0% and up to at most about 75% or greater than 0% and up to at most about 70%, between about 30% and about 90%, or between about 30% and about 88%, or between about 30% and about 86%, or between about 30% and about 84%, or between about 30% and about 82%, or between about 30% and about 80%, or between 34% and 90%, or between about 34% and about 88%, or between about 34% and about 86%, or between about 34% and about 84%, or between about 34% and about 82%, or between about 34% and about 80%, or between about 36% and about 90%, or between about 36% and about 88%, or between about 36% and about 86%, or between about 36% and about 84%, or between about 36% and about 82% or between about 36% and about 80%, or between about 40% and about 90%, or between about 40% and about 85%, or between about 45% and about 90%, or between about 45% and about 85%, or between about 50% and about 90%, or between about 50% and about 85%, or between about 55% and about 90%, or between about 55% and about 85%, or between about 60% and about 90%, or between about 60% and about 85%, or between about 65% and about 90%, or between about 65% and about 85%, or between about 70% and about 90%, or between about 70% and about 85%; (iii) RET may be greater than 0% and up to at most about 94%, or greater than 0% and up to at most about 93%, or greater than 0% and up to at most about 92%, or greater than 0% and up to at most about 91%, or greater than 0% and up to at most about 90%, or greater than 0% and up to at most about 89%, or greater than 0% and up to at most about 88%, or greater than 0% and up to at most about 87%, or greater than 0% and up to at most about 86% or greater than 0% and up to at most about 85%, or between about 45% and about 95%, or between about 45% and about 94%, or between about 45% and about 93%, or between about 45% and about 92%, or between about 45% and about 91%, or between about 45% and about 90%, or between about 45% and about 89%, or between about 45% and about 88%, or between about 47% and about 95%, or between about 47% and about 94%, or between about 47% and about 93%, or between about 47% and about 92%, or between about 47% and about 91%, or between about 47% and about 90%, or between about 47% and about 89%, or between about 47% and about 88%, or between about 49% and about 95%, or between about 49% and about 94%, or between about 49% and about 93%, or between about 49% and about 92%, or between about 49% and about 91%, or between about 49% and about 90%, or between about 49% and about 89% or between about 49% and about 88%, or between about 20% to about 60%, or between about 20% to about 55%, or between about 20% to about 50%, or between about 20% to about 45%, or between about 20% to about 40%, or between about 25% to about 60%, or between about 25% to about 55%, or between about 25% to about 50%, or between about 25% to about 45%, or between about 25% to about 40%; and (iv) podocin may be at least about 80%, at least about 82%, at least about 84%, at least about 86%, at least about 88%, at least about 90%, at least about 92%, at least about 94%, at least about 96% or at least about 98%.

In the methods of identifying an enriched heterogeneous renal cell population as having therapeutic potential in which it is determined whether cells of the enriched heterogeneous renal cell population express SIX2, OSR1, RET and podocin, it may further be determined whether cells of the enriched heterogeneous renal cell population express one or more of LHX1, FGF8, RACK-1 and nephrin. In these methods, if it is determined that cells of the enriched heterogeneous renal cell population express SIX2, OSR, RET and podocin, and it is further determined that cells of the enriched heterogeneous population express one or more of LHX1, FGF8, RACK-1 and nephrin, the enriched heterogeneous renal cell population may be identified as having therapeutic potential.

In these methods, the determination of whether cells of the enriched heterogeneous renal cell population express SIX2, OSR1, RET and podocin, and further determination of whether cells of the enriched heterogeneous renal cell population express one or more of LHX1, FGF8, RACK-1 and nephrin, may be a determination of whether cells of the enriched heterogeneous renal cell population express any of the following: SIX2, OSR1, RET, podocin and LHX1; or SIX2, OSR1, RET, podocin and FGF8; or SIX2, OSR1, RET, podocin and RACK-1; or SIX2, OSR1, RET, podocin and nephrin; or SIX2, OSR1, RET, podocin, LHX1 and FGF8; or SIX2, OSR1, RET, podocin, LHX1 and RACK-1; or SIX2, OSR1, RET, podocin, LHX1 and nephrin; or SIX2, OSR1, RET, podocin, FGF8 and RACK-1; or SIX2, OSR1, RET, podocin, FGF8 and nephrin; or SIX2, OSR1, RET, podocin, RACK-1 and nephrin; or SIX2, OSR1, RET, podocin, LHX1, FGF8, and RACK-1; or SIX2, OSR1, RET, podocin, LHX1, FGF8 and nephrin; or SIX2, OSR1, RET, podocin, LHX, RACK-1 and nephrin; or SIX2, OSR1, RET, podocin, FGF8, RACK-1 and nephrin; or SIX2, OSR1, RET, podocin, LHX, FGF8, RACK-1 and nephrin.

The determination of whether cells of the enriched heterogeneous renal cell population express SIX2, OSR1, RET and podocin, and further one or more of LHX1, FGF8, RACK-1 and nephrin may be a determination of percent of cells that express SIX2, OSR1, RET, podocin, and further express one or more of LHX1, FGF8, RACK-1 and nephrin. If the determining that cells of the enriched heterogeneous renal cell population express SIX2, OSR, RET and podocin, and further one or more of LHX1, FGF8, RACK-1 then the percentage of cells of the population that express: (i) SIX2 may be greater than 0% and up to at most 15%, or may be greater than 0% and up to at most about 10.0%, or greater than 0% and up to at most about 9.5%, or greater than 0% and up to at most about 9.0%, or greater than 0% and up to at most about 8.5%, or greater than 0% and up to at most about 8.0%, or greater than 0% and up to at most about 7.5%, or greater than 0% and up to at most about 7.0%, or greater than 0% and up to at most about 6.5%, or greater than 0% and up to up to at most about 6.0%, or between about 0.02% to about 15.0%, or between about 0.02% to about 10.0%, or between about 0.02% to about 9.0%, or between about 0.02% and about 8.0%, or between about 0.02% and about 7.0%, or between about 0.02% and about 6.0%, or between about 0.04% and about 15%, or between about 0.04% to about 10.0%, or between about 0.04% to about 9.0%, or between about 0.04% and about 8.0%, or between about 0.04% and about 7.0%, or between about 0.04% and about 6.0%, or between about 1.0% to about 15.0%, or between about 1.0% to about 10.0%, or between about 1.0% to about 9.0%, or between about 1.0% and about 8.0%, or between about 1.0% and about 7.0% or between about 1.0% and about 6.0%; (ii) OSR1 may be greater than 0% and up to at most about 90%, or greater than 0% and up to at most about 88%, or greater than 0% and up to at most about 86%, or greater than 0% and up to at most about 84%, or greater than 0% and up to at most about 82%, or greater than 0% and up to at most about 80%, or greater than 0% and up to at most about 75% or greater than 0% and up to at most about 70%, between about 30% and about 90%, or between about 30% and about 88%, or between about 30% and about 86%, or between about 30% and about 84%, or between about 30% and about 82%, or between about 30% and about 80%, or between 34% and 90%, or between about 34% and about 88%, or between about 34% and about 86%, or between about 34% and about 84%, or between about 34% and about 82%, or between about 34% and about 80%, or between about 36% and about 90%, or between about 36% and about 88%, or between about 36% and about 86%, or between about 36% and about 84%, or between about 36% and about 82% or between about 36% and about 80%, or between about 40% and about 90%, or between about 40% and about 85%, or between about 45% and about 90%, or between about 45% and about 85%, or between about 50% and about 90%, or between about 50% and about 85%, or between about 55% and about 90%, or between about 55% and about 85%, or between about 60% and about 90%, or between about 60% and about 85%, or between about 65% and about 90%, or between about 65% and about 85%, or between about 70% and about 90%, or between about 70% and about 85%; (iii) RET may be greater than 0% and up to at most about 94%, or greater than 0% and up to at most about 93%, or greater than 0% and up to at most about 92%, or greater than 0% and up to at most about 91%, or greater than 0% and up to at most about 90%, or greater than 0% and up to at most about 89%, or greater than 0% and up to at most about 88%, or greater than 0% and up to at most about 87%, or greater than 0% and up to at most about 86% or greater than 0% and up to at most about 85%, or between about 45% and about 95%, or between about 45% and about 94%, or between about 45% and about 93%, or between about 45% and about 92%, or between about 45% and about 91%, or between about 45% and about 90%, or between about 45% and about 89%, or between about 45% and about 88%, or between about 47% and about 95%, or between about 47% and about 94%, or between about 47% and about 93%, or between about 47% and about 92%, or between about 47% and about 91%, or between about 47% and about 90%, or between about 47% and about 89%, or between about 47% and about 88%, or between about 49% and about 95%, or between about 49% and about 94%, or between about 49% and about 93%, or between about 49% and about 92%, or between about 49% and about 91%, or between about 49% and about 90%, or between about 49% and about 89% or between about 49% and about 88%, or between about 20% to about 60%, or between about 20% to about 55%, or between about 20% to about 50%, or between about 20% to about 45%, or between about 20% to about 40%, or between about 25% to about 60%, or between about 25% to about 55%, or between about 25% to about 50%, or between about 25% to about 45%, or between about 25% to about 40%; (iv) podocin may be at least about 80%, at least about 82%, at least about 84%, at least about 86%, at least about 88%, at least about 90%, at least about 92%, at least about 94%, at least about 96% or at least about 98%; and, optionally (v) LHX1 may be at least about 5%, at least about 6%, or at least about 7%, or at least about 8%, or at least about 9% or at least about 10%, greater than 0% and up to at most about 80%, or greater than 0% and up to at most about 70%, or greater than 0% and up to at most about 65%, or greater than 0% and up to at most about 64%, or greater than 0% and up to at most about 63%, or greater than 0% and up to at most about 62%, or greater than 0% and up to at most about 61%, or greater than 0% and up to at most about 60%, or greater than 0% and up to at most about 59%, or greater than 0% and up to at most about 58%, or greater than 0% and up to at most about 57%, or greater than 0% and up to at most about 56% or greater than 0% and up to at most about 55%, between about 6% and about 60%, or between about 6% and about 59%, or between about 6% and about 58%, or between about 6% and about 57%, or between about 6% and about 56%, or between about 6% and about 55%, or between about 6% and about 54%, or between about 8% and about 60%, or between about 8% and about 59%, or between about 8% and about 58%, or between about 8% and about 57%, or between about 8% and about 56%, or between about 8% and about 55%, or between about 8% and about 54%, or between about 10% and about 60%, or between about 10% and about 59%, or between about 10% and about 58%, or between about 10% and about 57%, or between about 10% and about 56%, or between about 10% and about 55% or between about 10% and about 54%, or between about 16% and about 80%, or between about 16% and about 70%, or between about 16% and about 60%, or between about 16% and about 58%, or between about 16% and about 56%, or between about 16% and about 54%, or between about 16% and about 52%, or between about 16% and about 50%, or between 22% and about 60%, or between about 22% and about 58%, or between about 22% and about 56%, or between about 22% and about 54%, or between about 22% and about 52%, or between about 22% and about 50%, and, optionally (vi) FGF8 may be at least about 0.2%, least about 0.45%, or at least about 0.48%, or at least about 0.5%, or at least about 0.55%, or at least about 0.6%, or at least about 0.8%, or at least about 1.0%, or at least about 1.5%, or at least about 2.0% or at about 2.5%, greater than 0% and up to at most about 65% or greater than 0% and up to at most about 64%, or greater than 0% and up to at most about 63%, or greater than 0% and up to at most about 62%, or greater than 0% and up to at most about 61%, or greater than 0% and up to at most about 60%, or greater than 0% and up to at most about 59%, or at most about 58%, or greater than 0% and up to at most about 57%, or greater than 0% and up to at most about 56% or greater than 0% and up to at most about 55%, between about 0.3% and about 64%, or between about 0.3% and about 63%, or between about 0.3% and about 62%, or between about 0.3% and about 61%, or between about 0.3% and about 60%, or between about 0.3% and about 59%, or between about 0.3% and about 58%, or between about 0.3% and about 57%, or between about 0.4% and about 64%, or between about 0.4% and about 63%, or between about 0.4% and about 62%, or between about 0.4% and about 61%, or between about 0.4% and about 60%, or between about 0.4% and about 59%, or between about 0.4% and about 58%, or between about 0.4% and about 57%, or between about 0.5% and about 64%, or between about 0.5% and about 63%, or between about 0.5% and about 62%, or between about 0.5% and about 61%, or between about 0.5% and about 60%, or between about 0.5% and about 59%, or between about 0.5% and about 58% or between about 0.5% and about 57%, or between about 1% and about 64%, or between about 1% and about 54%, or between about 1% and about 44%, or between about 1% and about 34%, or between 1% and about 24%, or between about 2% and about 64%, or between about 2% and about 54%, or between about 2% and about 44%, or between about 2% and about 34%, or between 2% and about 24%, or between about 3% and about 64%, or between about 3% and about 54%, or between about 3% and about 44%, or between about 3% and about 34%, or between 3% and about 24%; and, optionally, (vii) RACK-1 may be at least about 80%, at least about 82%, at least about 84%, at least about 86%, at least about 88%, at least about 90%, at least about 92%, at least about 94%, at least about 96%, or at least about 98%; and, optionally, (viii) nephron may be at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, between about 4% and about 95%, or between about 10% and about 95%, or between about 15% and about 95%, or between about 20% and about 95%, or between about 25% and about 95%, and between about 30% and about 95%, or between 35% and about 95%, or between about 40% and about 95%, or between about 45% and 95%, or between about 50% and about 95%, or between about 55% and about 95%, or between about 60% and about 95%, or between about 65% and about 95%, or between 70% and about 95%, or between about 75% and about 95%, or between about 80% and about 95% or between about 85% and about 95%.

In some other methods of identifying an enriched heterogeneous renal cell population as having therapeutic potential, it may be determined whether cells of the enriched heterogeneous renal cell population express nephrin, podocin and LHX1. In such methods, the enriched heterogeneous renal cell population may be identified as having therapeutic potential if cells of the enriched heterogeneous renal cell population are determined to express nephrin, podocin and LHX1.

The determining that cells of the enriched heterogeneous renal cell population express nephrin, podocin and LHX1 may include a determining of percentage of cells of the enriched heterogeneous renal cell population that express nephrin, podocin and LHX1. If the determining includes determining of a percentage of cells of the enriched heterogeneous renal cell population that express nephrin, podocin and LHX1, then to identify the enriched heterogeneous renal cell population as having therapeutic potential, the percentage of cells of the population that express: (i) nephrin may be at least about 70%, at least about 72%, at least about 75%, at least about 77%, at least about 80%, at least about 82%, at least about 85%, at least about 87%, at least about 90%, at least about 92%, at least about 95%, or at least about 97%; (ii) podocin may be at least about 80%, at least about 82%, at least about 84%, at least about 86%, at least about 88%, at least about 90%, at least about 92%, at least about 94%, at least about 96% or at least about 98%; and (iii) LHX1 may be at least about 15%, at least about 20%, at least about 25%, at least about 30%, at least about 35%, at least about 40%, at least about 45%, at least about 50%, at least about 55%, at least about 60%, at least about 65%, at least about 70%, between about 15% and about 80%, between about 20% and about 80%, between about 15% and about 75%, between about 15% and about 70%, or between about 20% and about 80%.

In the methods of identifying an enriched heterogeneous renal cell population as having therapeutic potential, in which it is determined whether cells of the enriched heterogeneous renal cell population express nephrin, podocin and LHX1, it may further be determined whether cells of the enriched heterogeneous renal cell population express one or more of nephrogenic markers SIX2, OSR1, RET or FGF8. In these methods, if it is determined that cells of the enriched heterogeneous renal cell population express nephrin, podocin and LHX1, and it is further determined that cells of the enriched heterogeneous population express one or more of SIX2, OSR1, RET or FGF8, then the enriched heterogeneous renal cell population may be identified as having therapeutic potential.

In these methods, the determination of whether cells of the enriched heterogeneous renal cell population express nephrin, podocin and LHX1, and further express one or more of SIX2, OSR1, RET or FGF8, may be a determination of whether cells of the enriched heterogeneous renal cell population express any of: nephrin, podocin, LHX1 and SIX2; nephrin, podocin, LHX1 and OSR1, nephrin, podocin, LHX1 and RET; nephrin, podocin, LHX1 and FGF8; nephrin, podocin, LHX1, SIX2 and OSR1; nephrin, podocin, LHX1, SIX2 and RET; nephrin, podocin, LHX1, SIX2 and FGF8; nephrin, podocin, LHX1, OSR1 and RET; nephrin, podocin, LHX1, ORS1 and FGF8; nephrin, podocin, LHX1, RET and FGF8; nephrin, podocin, LHX1, SIX2, OSR1 and RET; nephrin, podocin, LHX1, SIX2, RET and FGF8; nephrin, podocin, LHX1, OSR1, RET and FGF8; nephrin, podocin, LHX1, SIX2, OSR1 and FGF8; or nephrin, podocin, LHX1, SIX2, OSR1, RET and FGF8.

The determination of whether cells of the enriched heterogeneous renal cell population express nephrin, podocin and LHX1, and further express one or more of SIX2, OSR1, RET and FGF8 may be a determination of percent of cells that express nephrin, podocin and LHX1, and further express one or more of SIX2, OSR1, RET and FGF8. If the percentage of cells of the population that express nephrin, podocin and LHX1, and one or more of SIX2, OSR1, RET and FGF8 is determined, then to identify the enriched heterogeneous renal cell population as having therapeutic potential, then the percentage of cells that express: (i) nephrin may be at least about 70%, at least about 72%, at least about 75%, at least about 77%, at least about 80%, at least about 82%, at least about 85%, at least about 87%, at least about 90%, at least about 92%, at least about 95%, or at least about 97%; (ii) podocin may be at least about 80%, at least about 82%, at least about 84%, at least about 86%, at least about 88%, at least about 90%, at least about 92%, at least about 94%, at least about 96% or at least about 98%; (iii) LHX1 may be at least about 15%, at least about 20%, at least about 25%, at least about 30%, at least about 35%, at least about 40%, at least about 45%, at least about 50%, at least about 55%, at least about 60%, at least about 65%, at least about 70%, between about 15% and about 80%, between about 20% and about 80%, between about 15% and about 75%, between about 15% and about 70%, or between about 20% and about 80%; and, optionally, (iv) SIX2 may be may be greater than 0% and up to at most about 15.0%, or greater than 0% and up to at most about 13%, or greater than 0% and up to at most about 11%, or greater than 0% and up to at most about 9%, or greater than 0% and up to at most about 7%, or greater than 0% and up to at most about 5%, or greater than 0% and up to at most about 3%, or between about 0.02% to about 15%, or between about 0.02% to about 13%, or between about 0.02% and about 11%, or between about 0.02% and about 9%, or between about 0.02% and about 7%, or between about 0.02% and about 5%, or between about 0.02% and about 3%, or between about 0.04% to about 15%, or between about 0.04% to about 13%, or between about 0.04% and about 11%, or between about 0.04% and about 9%, or between about 0.04% and about 7%, or between about 0.04% and about 5%, or between about 0.04% and about 3%, or between about 1% to about 15%, or between about 1% to about 13%, or between about 1% and about 11%, or between about 1% and about 9%, or between about 1% and about 7%, or between about 1% and about 5%, or between about 1% and about 3%; and, optionally, (v) OSR1 may be greater than 0% and up to at most about 90%, or greater than 0% and up to at most about 88%, or greater than 0% and up to at most about 86%, or greater than 0% and up to at most about 84%, or greater than 0% and up to at most about 82%, or greater than 0% and up to at most about 80%, or greater than 0% and up to at most about 75% or greater than 0% and up to at most about 70%, between about 30% and about 90%, or between about 30% and about 88%, or between about 30% and about 86%, or between about 30% and about 84%, or between about 30% and about 82%, or between about 30% and about 80%, or between 34% and 90%, or between about 34% and about 88%, or between about 34% and about 86%, or between about 34% and about 84%, or between about 34% and about 82%, or between about 34% and about 80%, or between about 36% and about 90%, or between about 36% and about 88%, or between about 36% and about 86%, or between about 36% and about 84%, or between about 36% and about 82% or between about 36% and about 80%, or between about 40% and about 90%, or between about 40% and about 85%, or between about 45% and about 90%, or between about 45% and about 85%, or between about 50% and about 90%, or between about 50% and about 85%, or between about 55% and about 90%, or between about 55% and about 85%, or between about 60% and about 90%, or between about 60% and about 85%, or between about 65% and about 90%, or between about 65% and about 85%, or between about 70% and about 90%, or between about 70% and about 85%; and, optionally, (vi) RET may be greater than 0% and up to at most about 94%, or greater than 0% and up to at most about 93%, or greater than 0% and up to at most about 92%, or greater than 0% and up to at most about 91%, or greater than 0% and up to at most about 90%, or greater than 0% and up to at most about 89%, or greater than 0% and up to at most about 88%, or greater than 0% and up to at most about 87%, or greater than 0% and up to at most about 86% or greater than 0% and up to at most about 85%, or between about 45% and about 95%, or between about 45% and about 94%, or between about 45% and about 93%, or between about 45% and about 92%, or between about 45% and about 91%, or between about 45% and about 90%, or between about 45% and about 89%, or between about 45% and about 88%, or between about 47% and about 95%, or between about 47% and about 94%, or between about 47% and about 93%, or between about 47% and about 92%, or between about 47% and about 91%, or between about 47% and about 90%, or between about 47% and about 89%, or between about 47% and about 88%, or between about 49% and about 95%, or between about 49% and about 94%, or between about 49% and about 93%, or between about 49% and about 92%, or between about 49% and about 91%, or between about 49% and about 90%, or between about₄₉% and about 89% or between about 49% and about 88%, or between about 20% to about 60%, or between about 20% to about 55%, or between about 20% to about 50%, or between about 20% to about 45%, or between about 20% to about 40%, or between about 25% to about 60%, or between about 25% to about 55%, or between about 25% to about 50%, or between about 25% to about 45%, or between about 25% to about 40%; and, optionally, (vii) FGF8 may be at least about 0.2%, least about 0.45%, or at least about 0.48%, or at least about 0.5%, or at least about 0.55%, or at least about 0.6%, or at least about 0.8%, or at least about 1.0%, or at least about 1.5%, or at least about 2.0% or at least about 2.5%, greater than 0% and up to at most about 65%, or greater than 0% and up to at most about 60%, or greater than 0% and up to at most about 59%, or at most about 58%, or greater than 0% and up to at most about 57%, or greater than 0% and up to at most about 56% or greater than 0% and up to at most about 55%, between about 0.3% and about 64%, or between about 0.3% and about 63%, or between about 0.3% and about 62%, or between about 0.3% and about 61%, or between about 0.3% and about 60%, or between about 0.3% and about 59%, or between about 0.3% and about 58%, or between about 0.3% and about 57%, or between about 0.4% and about 64%, or between about 0.4% and about 63%, or between about 0.4% and about 62%, or between about 0.4% and about 61%, or between about 0.4% and about 60%, or between about 0.4% and about 59%, or between about 0.4% and about 58%, or between about 0.4% and about 57%, or between about 0.5% and about 64%, or between about 0.5% and about 63%, or between about 0.5% and about 62%, or between about 0.5% and about 61%, or between about 0.5% and about 60%, or between about 0.5% and about 59%, or between about 0.5% and about 58% or between about 0.5% and about 57%, or between about 1% and about 64%, or between about 1% and about 54%, or between about 1% and about 44%, or between about 1% and about 34%, or between 1% and about 24%, or between about 2% and about 64%, or between about 2% and about 54%, or between about 2% and about 44%, or between about 2% and about 34%, or between 2% and about 24%, or between about 3% and about 64%, or between about 3% and about 54%, or between about 3% and about 44%, or between about 3% and about 34%, or between 3% and about 24%.

In the methods of identifying an enriched heterogeneous renal cell population as having therapeutic potential, in which it is determined whether cells of the enriched heterogeneous renal cell population express nephrin, podocin and LHX1, it may further be determined whether cells of the enriched heterogeneous renal cell population express RACK1, in addition or as an alternative to, determining whether the cells express one or more of nephrogenic markers SIX2, OSR1, RET or FGF8. In these methods, the enriched heterogeneous renal cell population may be identified as having therapeutic potential if cells of the heterogeneous renal cell population express nephrin, podocin and LHX1, and further express RACK-1.

In these methods, the determination of whether cells of the enriched heterogeneous renal cell population express RACK1 may be a determination of percentage of cells of the enriched heterogeneous renal cell population that express RACK1, in addition to percentage of cells that express nephrin, podocin and LHX1, and optionally percentage of cells that express one or more of SIX1, OSR1, RET and FGF8. If the percentage of cells of the enriched heterogeneous renal cell population express RACK1 is determined as part of the method, then the percentage that identifies the enriched renal cell population as having therapeutic potential may be at least about 80%, at least about 82%, at least about 84%, at least about 86%, at least about 88%, at least about 90%, at least about 92%, at least about 94%, at least about 96%, or at least about 98%.

In yet another method of identifying an enriched heterogeneous renal cell population as having therapeutic potential, it may be determined whether cells of the enriched heterogeneous renal cell population express gamma-glutamyl transpeptidase (GGT)-1, CK18 and podocin. In such methods, the enriched heterogeneous renal cell population may be identified as having therapeutic potential if cells of the enriched heterogeneous renal cell population are determined to express GGT-1, CK18 and podocin. The determining that cells of the enriched heterogeneous renal cell population express GGT-1 CK18 and podocin may include a determining of a percentage of cells of the enriched heterogeneous renal cell population that express GGT-1, CK18 and podocin. If the determining that cells of the enriched heterogeneous renal cell population express GGT-1, CK18 and podocin, then the enriched heterogeneous renal cell population may be identified as having therapeutic potential if: (i) at least 4.5% or at least 10% or at least 18% of cells of the population express GGT-1, (ii) at least 80% of cells of the population express CK18, and (iii) at least about 80%, at least about 82%, at least about 84%, at least about 86%, at least about 88%, at least about 90%, at least about 92%, at least about 94%, at least about 96% or at least about 98% of cells of the population express podocin. In these methods, VEGF and/or KIM-1 secreted in the cell culture media by cells of the population may further identify the cells as having therapeutic potential.

In some methods of identifying an enriched heterogeneous renal cell population as having therapeutic potential, it is not necessary to determine whether cells of the enriched heterogeneous renal cell population express at least one nephrogenic marker. In these, alternative, methods of identifying an enriched heterogeneous renal cell population as having a therapeutic potential, the cells may be identified as having therapeutic potential by determining expression level of one or more of genes RHAMM, C2, C3, C4, fibrinogen, coagulation factor XIII, TEK, KDR, Notch1, Notch3, Timp3, Vwf, Adam15, Gas6, Igfbp1, or Tm4sf4. In these alternative methods, the one or more genes whose expression level may be determined may be RHAMM. In these alternative methods, the enriched heterogeneous renal cell population may be identified as having therapeutic potential if the determined expression level of the one or more genes by cells of the enriched heterogeneous renal cell population is increased relative to expression of the one or more genes by cells of a control renal cell population.

Further, in any of the methods of identifying an enriched heterogeneous renal cell population as having therapeutic potential, the enriched heterogeneous renal cell population may be further subjected to transcriptional, or transcriptomic signature, analysis. If the enriched heterogeneous renal cell population is subjected to transcriptional, or transcriptomic signature, analysis, it may be identified as having therapeutic potential if it is determined that cells of the enriched heterogeneous renal cell population are up-regulated for transcriptional pathways or signatures relating complement or complement cascades, blood vessel development, blood vessel morphogenesis, vasculature development or response to wounding and/or are down-regulated for transcriptional pathways or signatures relating to extracellular matrix-receptor interaction.

Markers useful to identify an enriched heterogenous renal cell population as having therapeutic potential as discussed herein, e.g., SIX2, OSR1, RET, LHX1, FGF8, nephrin, podocin and RACK-1, may also be useful in methods of identifying whether a patient in need of treatment with the enriched heterogeneous renal cell population, e.g., patient in need of treatment for a kidney disease, a tubular transport deficiency, or a glomerular filtration deficiency, will be a moderate to high responder or a low responder to the treatment. In such methods, if a patient is identified as a moderate to high responder, the patient may be identified as one whose response to the treatment may be an increase in estimated glomerular filtration rate (eGFR). If the patient is identified as a low responder, the patient may be identified as one whose response to the treatment may be an improved slope of eGFR, but without an increase in eGFR. To identify whether the patient may be a moderate to high responder or a low responder, percentage of cells of the enriched heterogeneous renal cell population that express the one or more of markers, e.g., SIX2, OSR1, RET, LHX1, FGF8, nephrin, podocin and RACK-1, may be determined. For example, to identify whether the patient is a moderate to high responder or a low responder, the percentage of cells of the enriched heterogeneous renal cell population that express LHX1 may be determined. If the percentage of cells of the heterogeneous renal cell population that express LHX1 is determined to be at least 50%, the patient may be identified as a moderate to high responder. If the percentage of cells of the heterogeneous renal cell population that express LHX1 is determined to be less than 50%, the patient may be identified as a low responder. By way of another example, to identify whether the patient is a moderate to high responder or a low responder, the percentage of cells of the enriched heterogeneous renal cell population that express SIX2 may be determined. If the percentage of cells of the heterogeneous renal cell population that express SIX2 is determined to be at least 3.5%, the patient may be identified as a moderate to high responder. If the percentage of cells of the heterogeneous renal cell population that express SIX2 are determined to be less than 3.5% but greater than 0%, the patient may be identified as a low responder. Percentages of both LHX1 and SIX2 may be determined to identify the patient as a moderate to high responder or a low responder.

In any of the methods of identifying an enriched heterogeneous renal cell population as having a therapeutic potential, the determining whether cells of the enriched heterogeneous renal cell population express the one or more, e.g., nephrogenic and/or further, marker (or the determining expression level of the one or more genes) may be a determining whether the cells of the enriched heterogeneous renal cell population express the marker (or may be a determining expression level of the one or more genes) in a nucleic acid, e.g., mRNA or miRNA, or polypeptide form. The marker (or gene whose expression or expression level is determined) may be membrane bound or membrane associated, it may be intracellular or it may be secreted from the cells. The expression of the one or more, e.g., nephrogenic and/or further, marker (or the expression level of the gene) by cells of the enriched heterogeneous renal cell population may be determined via any assay suitable for detecting presence of the marker (or level of expression of the gene). Many such assays are known in the art. For example, if the marker (or the gene's expression level is determined and the expression) is determined in polypeptide form, it may be determined by assays such as Western blot, fluorescence activated cell sorting (FACS), enzyme linked immunosorbent assay (ELISA). If the marker (or the gene's expression level is determined and the expression) is determined in nucleic acid form, it may be determined by assays such as Southern blot, polymerase chain reaction (PCR) or reverse transciptase PCR, serial analysis of gene expression (SAGE), Mass ARRAY, or fluorescence in situ hybridization (FISH). Regardless of whether the assay determines whether the nephrogenic and/or further, marker is expressed (or determines expression level of the one or more genes) by cells of the enriched heterogeneous renal cell population, the assay may include a labeled detection reagent for determining whether the marker (or gene whose expression level is determined) is present and/or percent of cells that express the marker (or gene). The labeled detection reagent may include (i) a portion that complexes, directly or indirectly, with the marker (or expression product of the gene) and (ii) a detection moiety. Non-limiting detection moieties include radioactive isotopes, e.g., ³⁵S, ¹⁴C, ¹²⁵I, ³H and ¹³¹I, colloidal gold particles, fluorescent labels, e.g., Texas Red, rhodamine, fluorescein, dansyl, Lissamine, phycocryterin, phycocyanin, SPECTRUM ORANGE, SPECTRUM GREEN1 and enzyme substrates, e.g., firefly luciferase, bacterial luciferase, luciferin, horseradish peroxidase, alkaline phosphatase, or beta galactosidase.

The enriched heterogeneous renal cell population, which may be identified as having a therapeutic potential in any of the methods, may be enriched for one or more renal cell types such as renal epithelial cells, renal tubular cells, renal tubular epithelial cells, or renal proximal tubular cells. The enrichment of the enriched heterogeneous renal cell population for these one or more of renal cell types may be a reference to the enriched heterogeneous renal cell population having a greater percentage of the one or more renal cell types than does a kidney tissue of a patient, a kidney biopsy of a patient, or an in vitro culture of cells established from a kidney tissue or kidney biopsy of a patient, (which, collectively, may be referred to as a “starting renal cell population”). A starting renal cell population, if an in vitro culture of cells established from a kidney tissue of a patient or a kidney biopsy of a patient, may be a renal cell preparation comprising dissociated cells of a kidney tissue or kidney biopsy (e.g., cells dissociated from the kidney tissue or kidney biopsy via mincing and/or enzyme digestion), that may or may not have been treated to remove red blood cells and debris. An example of an enriched heterogeneous renal cell population is a selected renal cell population (SRC) as described in the Examples herein.

The enriched heterogeneous renal cell population may be enriched for the one or more renal cell types as a result of having been prepared from a starting renal cell population, (e.g., a kidney tissue of a patient, a kidney biopsy of a patient, or an in vitro culture of cells established from a kidney tissue or kidney biopsy of a patient), via a method that includes a separation step. The separation step may be one that separates cells of the starting renal cell population that have passaged no more than one, two, or three times, on the basis of their buoyant density. If the separation step is one that separates cells on the basis of their buoyant density, the separation step may utilize a single or multi-step continuous or discontinuous density gradient using a density gradient media such as glycerol, glucose OptiPrep, Percoll, or Ficoll-Paque. The use of such a density gradient media in this manner may result in cells of the starting renal cell population (or starting renal cell population having been passaged at most one, two or three times) separating into one or more distinguishable fractions from which cells of the enriched heterogeneous renal cell population may be distinctly identified and isolated. The distinguishable fraction(s) may be those in which the buoyant density of cells in the fraction(s) is greater than about 1.045 g/mL, or greater than 1.045 g/mL, or greater than or equal to 1.045 g/mL. The distinguishable fraction(s) may be those in which the buoyant density of cells in the fraction(s) is greater than about 1.04 g/mL, or greater than 1.04 g/mL, or greater to or equal than 1.04 g/mL, or greater than about 1.0419 g/mL, or greater than 1.0419 g/mL, or greater to or equal than 1.0419 g/mL. The distinguishable fraction(s) may be those in which the buoyant density is between about 1.045 g/mL and about 1.091 g/mL, or between about 1.045 g/mL and about 1.052 g/mL. Alternatively, the separation step may be one that separates cells of the starting renal cell population (or cells of the starting renal cell population that have been passaged no more than one, two or three times), on the basis of whether they express particular markers on their surface. If the separation step separates cells on the basis of their expression of particular cell surface markers, the separation step may be one that utilizes flow cytometry. The flow cytometry may sort out cells from the starting renal cell population (or starting renal cell population having been passaged at most one, two or three times) if they express particular surface markers, such as nephrin, characteristic of, e.g., renal epithelial cells, renal tubular cells, renal tubular epithelial cells, or renal proximal tubular cells, to thereby form the isolated enriched heterogeneous renal cell population.

The enriched heterogeneous renal cell population, having been prepared from a starting renal cell population (or starting renal cell population having been passaged at most one, two or three times) may be cultured under hypoxic conditions prior to the separation step. If the cells are cultured under hypoxic conditions prior to the separation step, the cells may be cultured under conditions in which the oxygen levels are less than about 20%, or less than about 15%, or less than about 10%, or less than about 9%, or less than about 8%, or less than about 7%, or less than about 6%, or less than about 5% oxygen, or less than about 4% oxygen, or less than about 3% oxygen or less than about 2% oxygen. If the cells are cultured under hypoxic conditions, the cells may be cultured under the hypoxic conditions for at least 6 hours, at least 8 hours, at least 10 hours, at least 12 hours, at least 14 hours, at least 16 hours, at least 20 hours, at least 24 hours, at least 30 hours, at least 36 hours, at least 42 hours or at least 48 hours.

In general, the preparation of an enriched heterogeneous renal cell population may be from any starting cell population, for example, an in vitro culture of cells established from a kidney tissue of a patient or a kidney biopsy of a patient. If the enriched heterogeneous renal cell population is prepared from the in vitro culture of cells established from the kidney tissue or kidney biopsy of the patient, the cells of the in vitro culture may be expanded by passaging at most one, or at most two or at most three times. Alternatively, if desired, cells of the in vitro culture of cells established from the kidney tissue or kidney biopsy may be cryopreserved and then expanded by passaging at most one, or at most two or at most three times. Once the cells have been expanded, the expanded cells may be cryopreserved. The expanded cells, whether or not cryopreserved, may then be subject to a separation step or may then be subject to hypoxic culture conditions followed by a separation step. The enriched heterogeneous renal cell population is isolatable by having performed the separation step. Once the enriched renal cell population has been isolated, it may be frozen and/or analyzed prior to use as a therapeutic.

Further, in the preparation of the enriched heterogeneous renal cell population from the starting renal cell population, one or more control renal cell populations may be generated. A control renal cell population may be any renal cell population generated as a result of subjecting the starting renal cell population to one or more steps or conditions, prior to the separation step, in the preparation of the enriched heterogeneous renal cell population. A control renal cell population may be a renal cell population generated from the starting renal cell population following subjecting the starting renal cell population to an expansion step, e.g., by passaging the starting renal cell population at most one, two or three times, prior to the separation step. Alternatively, the control renal cell population may be a renal cell population generated from the starting renal cell population following culturing the starting renal cell population under hypoxic conditions, prior to the separation step. In another example, the control renal cell population may be a renal cell population generated from the starting renal cell population as a result of the starting renal population having been passaged at most one, two or three times and having been cultured under hypoxic conditions, prior to performance of the separation step.

In yet another example, the control renal cell population may be a renal cell population generated from the starting renal cell population as a result of the starting renal cell population having been expanded by passaging at most one, two or three times and/or having been cultured under hypoxic conditions and/or having been cryopreserved, prior to performance of the separation step. A control renal cell population may be a renal cell population generated from a starting renal cell population that has been subjected to a complete set of steps or conditions in preparation for performing the separation step, but that has not yet been subject to the separation step.

Such control renal cell populations may be used in methods provided herein in which an enriched heterogeneous renal cell population may be identified as having therapeutic potential if expression level of one or more genes, e.g., RHAMM, C2, C3, C4, fibrinogen, coagulation factor XIII, TEK, KDR, Notch1, Notch3, Timp3, Vwf, Adam15, Gas6, Igfbp1, or Tm4sf4, by cells of the enriched heterogeneous renal cell population is increased relative to level of expression of the one or more genes by cells of a control renal cell population. The increased level in expression of the one or more genes may be an increase of at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In such methods, the one or more genes may be, or may include, RHAMM and the increase RHAMM expression level in the enriched heterogeneous renal cell population relative to the control population may be an increase of at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In such methods, an increase in expression level may be an increase in percentage of cells that express the one or more gene or may be an increase in total quantity of expression (adjusted for number of cells) of the one or more gene.

If the enriched heterogeneous renal cell population is identified as having a therapeutic potential in accordance with any of the methods disclosed herein, it may be included in a pharmaceutical composition, or administered in a method of treating kidney disease in a patient in need thereof, and/or used in the manufacture of a medicament to treat kidney disease. If the enriched heterogeneous renal cell population is identified as having a therapeutic potential and is included in a pharmaceutical composition, it may formulated as a hydrogel composition or as a liquid composition. It may, or may not, include hyaluronic acid.

If the pharmaceutical composition is formulated as a hydrogel composition, cells of the enriched heterogeneous renal cell composition may be combined with a temperature-sensitive cell-stabilizing biomaterial that maintains a gel state at about 8° C. or below, maintains a substantially liquid state at about ambient temperature or above, and is in a solid-to-liquid transitional state between about 8° C. and about ambient temperature or above. The temperature-sensitive cell-stabilizing biomaterial comprised in the hydrogel composition may be one or more of an extracellular matrix protein of recombinant origin, an extracellular matrix sourced from kidney or another tissue or organ, or gelatin. If the temperature-sensitive cell-stabilizing biomaterial includes gelatin, the gelatin may be derived from a Type I, alpha I collagen such as porcine Type I, alpha I collagen or recombinant human Type I, alpha I collagen. If the temperature-sensitive cell-stabilizing biomaterial comprises gelatin, the gelatin may present in the pharmaceutical composition at about 0.5% to about 1% (w/v) or about 0.8% to about 0.9% (w/v) or about 0.88% (w/v). Cells of the enriched heterogeneous renal cell population may be dispersed throughout the biomaterial, or substantially uniformly distributed throughout the biomaterial.

If the pharmaceutical composition is formulated as a liquid composition, the enriched heterogeneous renal cell population may be combined with any suitable liquid, e.g. appropriate cell storage or culture medium, a saline, or combinations thereof, for immediate use or for frozen storage up until the timing of its use.

If the enriched heterogeneous renal cell population is identified as having a therapeutic potential, the enriched heterogeneous renal cell population, or a pharmaceutical composition comprising the enriched heterogeneous renal cell population, may be administered to a patient in a method of treating kidney disease. If the enriched heterogeneous renal cell population is identified as having a therapeutic potential, the enriched heterogeneous renal cell population, or a pharmaceutical composition comprising the enriched heterogeneous renal cell population, may be used in the manufacture of a medicament to treat kidney disease. The kidney disease may be at any stage or degree of acute or chronic renal failure. It may originate in the kidney or it may secondary to another condition, e.g., heart failure, hypertension, diabetes, autoimmune disease or liver disease. Alternatively, the kidney disease may be a kidney disease arising from an acute injury to the kidney, or the result of an anomaly of the kidney and/or urinary tract. The kidney disease may further include endocrine dysfunctions such as anemia, e.g., erythropoietin-deficiency, and mineral imbalance, e.g., Vitamin D deficiency.

If the enriched heterogeneous real cell population is identified as having a therapeutic potential, then administering the enriched heterogeneous renal cell population, or a pharmaceutical composition comprising the enriched heterogeneous renal cell population, may treat the kidney disease. It may treat the kidney disease by restoring kidney function, stabilizing kidney function, improving kidney function, reducing renal fibrosis, reducing renal inflammation, inducing in a kidney of a patient in need of such treatment. The treating the kidney disease may restore mineral balance or alleviate anemia in a patient in need of such treatment. The treating the kidney disease may delay or prevent the need for dialysis, or it may delay or prevent the need for a kidney transplant in a patient in need of a treatment for kidney disease. If the treating the kidney disease delays the need for dialysis or the need for a kidney transplant in the patient, the delay may be by at least 1 year, at least 1.5 years, at least 2 years, at least 2.5 years, at least 3 years, at least 3.5 years, at least 4 years, at least 4.5 years, at least 5 years, at least 5.5 years, at least 6 years, at least 6.5 year, at least 7 years, at least 7.5 years, at least 8 years, at least 8.5 years, at least 9 years, at least 9.5 years or at least 10 years. The treating the kidney disease may be determined by observation in an improvement in the patient's serum albumin, albumin to globulin ratio (A/G ratio), serum phosphorous, serum sodium, kidney size (measurable by ultrasound), serum calcium, phosphorous:calcium ratio, serum potassium, proteinuria, urine creatinine, serum creatinine, blood nitrogen urea (BUN), cholesterol levels, triglyceride levels and glomerular filtration rate (GFR), weight, blood pressure (mean systemic blood pressure, diastolic blood pressure, or systolic blood pressure), and physical endurance performance.

If the enriched heterogeneous renal cell population is identified as having a therapeutic potential, it may be administered to a patient by any suitable administration route known in the art. For instance, the enriched heterogeneous renal cell population, or a pharmaceutical composition comprising the enriched heterogeneous renal cell population, may be systemically administered to a patient in need of treatment for kidney disease. The enriched heterogeneous renal cell population, or a pharmaceutical composition comprising the enriched heterogeneous renal cell population, may be administered at or into the kidney(s) of a patient in need of treatment for kidney disease. If the enriched heterogeneous renal cell population is administered at or into the kidney(s) of the patient in need of treatment for kidney disease, it may be administered over a single or over multiple injection(s). It may be administered via direct laparotomy, via direct laparoscopy, transabdominally, or percutaneously. The enriched heterogeneous renal cell population, or pharmaceutical composition comprising the enriched heterogeneous renal cell population, may be administered by percutaneous injection into the renal cortex of a kidney, or may be administered by inserting a guiding cannula percutaneously to puncture the kidney capsule and then injecting the enriched heterogeneous renal cell population into the kidney.

The enriched heterogeneous renal cell population, or pharmaceutical composition comprising the enriched heterogeneous renal cell population, is administered, by the any suitable route, at a therapeutically effective dose. A therapeutically effective dose, or amount, for administration to the patient in need of treatment for kidney disease may include about 1-9×10⁶ enriched heterogeneous renal cell population cells per gram estimated kidney weight of the patient. A therapeutically effective amount of the pharmaceutical composition may be a dose of about 1×10⁶ cells, 1×10⁶ cells, about 2×10⁶ cells, 2×10⁶ cells, about 3×10⁶ cells, 3×10⁶ cells, about 4×10⁶ cells, 4×10⁶ cells, about 5×10⁶ cells, or 5×10⁶ cells of an enriched heterogeneous renal cell population per gram estimated kidney weight of the patient.

Those skilled in the art will recognize, or be able to ascertain using no more than routine experimentation, many equivalents to the specific embodiments described herein. Such equivalents are intended to be encompassed by the appended claims.

All publications, patents and patent applications mentioned in this specification are herein incorporated by reference into the specification to the same extent as if each individual publication, patent or patent application was specifically and individually indicated to be incorporated herein by reference in their entirety.

### EXAMPLES

### Example 1: Preparation of an Enriched Heterogeneous Renal Cell Population

Solutions. In one method of preparing an enriched heterogeneous renal cell population, the reagents provided in Table 1 were used to prepare an enriched heterogeneous renal cell population from a kidney tissue or biopsy sample.

TABLE 1
Culture Media and Solutions

| Material | Composition |
|---|---|
| Tissue Transport Medium | Viaspan ™ or HypoThermosol-FRS ® |
| | or DMEM |
| | Kanamycin: 100 μg/mL |
| Renal Cell Growth Medium | DMEM:KSFM (50:50) |
| | 5% FBS |
| | Growth Supplements: |
| | HGF: 10 mg/L |
| | EGF: 2.5 μg/L |
| | Insulin: 10.0 mg/L |
| | Transferrin: 5.5 mg/L |
| | Selenium: 670 μg/L |
| | Kanamycin: 10 μg/L |
| Tissue Wash Solution | DMEM |
| | Kanamycin: 10 μg/mL |
| Digestion Solution | Collagenase IV: 300 Units |
| | Dispase: 5 mg/mL |
| | Calcium Chloride: 5 mM |
| Cell Dissociation Solution | TrypLE ™ |
| Density Gradient Solution | 7% OptiPrep |
| | OptiMEM |
| Cryopreservation Solution | DMEM or HypoThermosol - FRS |
| | 10% DMSO |
| | 10% FBS |

Dulbecco's Phosphate Buffered Saline (DPBS) is useful for all cell washes.

Isolating cells frm a kidney sample. In this method, renal tissue via kidney biopsy was used as a source material for arriving at a preparation of unenriched of renal cells. Generally, the renal tissue used in the preparation may be comprised of one or more of cortical, corticomedullary junction or medullary tissue. The corticomedullary junction tissue is preferred. Multiple biopsy cores (minimum 2), avoiding scar tissue, may be required from a CKD kidney. Renal tissue was obtained by a clinician from a patient. If necessary, tissue may be transported in Tissue Transport Medium.

The tissue was washed with Tissue Wash Solution in order to reduce incoming bioburden before processing the tissue for cell extractions.

Renal tissue was minced and dissociated in the Digestion Solution. The resulting cell suspension was neutralized, washed and resuspended in Dulbecco's Modified Eagle Medium (D-MEM)+10% fetal bovine serum (FBS) (Invitrogen, Carlsbad Calif.), washed, and resuspended in Renal Cell Growth Medium (RCGM). Initiation of culture onto tissue culture treated polystyrene flasks or dishes was done in RCGM. For example, one biopsy was plated into one T25 Nunc flask in 8 mL of RCGM.

Expansion of the unenriched heterogeneous renal culture. Renal cell expansion was dependent on the amount of tissue received and on the success of isolating renal cells from the incoming tissue. If required, isolated cells can be cryopreserved. Renal cell growth kinetics may vary from sample to sample due to the inherent variability of cells isolated from individual patients.

A defined cell expansion process has been developed and the process accommodates the range of cell recoveries resulting from the variability of incoming tissue. Table 2. Expansion of renal cells involved serial passages in closed culture vessels (e.g., T-flasks, Cell Factories, HyperStacks®) in Renal Cell Growth Medium using defined cell culture procedures.

TABLE 2
Renal Cell Yields from Human Biopsies

| | Renal cells | |
|---|---|---|
| | (cells/10 mg tissue) | |
| Source | (passage 0) | (passage 1) |
| Human Kidney Biopsy | 402,000 − 11.8 × 10⁶ | 2.21 − 42.735 × 10⁷ |
| Samples (n = 82) | | |

Once cell growth was observed in the initial T-flasks (passage 0) and there were no visual signs of contamination, culture medium was replaced and changed thereafter every 2-4 days. Cells were assessed to verify renal cell morphology by visual observation of cultures under the microscope. Cultures characteristically demonstrated a tight pavement or cobblestone appearance, due to the cells clustering together. These morphological characteristics can vary during expansion and may not be present at every passage. Cell culture confluence was estimated using an Image Library of cells at various levels of confluence in the culture vessels employed throughout cell expansions.

Renal cells were passaged by trypsinization when culture vessels were at least 50% confluent. Detached cells were collected into vessels containing Renal Cell Growth Medium, counted and cell viability was calculated. At each cell passage, cells were seeded at 500-4000 cells/cm² in a sufficient number of culture vessels in order to expand the cell number to one required for therapeutic formulation or evaluation. Culture vessels were placed in a 37° C. incubator in a 5% CO₂ environment. As described above, cell morphology and confluence were monitored and tissue culture media was replaced every 2-4 days. Table 3 lists the viability of human renal cells observed during cell isolation and expansion of kidney biopsies from human donors.

TABLE 3
Cell Viability of Human Renal Cells in Culture

| | Cell Viability | Range |
|---|---|---|
| Passage | (Average %) | (%) |
| P0 (n = 82) | 89.2 | 68.7-99.1 |
| P1 (n = 82) | 96.4 | 89.3-99.1 |
| P2 (n = 82) | 97.4 | 91.9-100 |
| P3 (n = 91) | 94.7 | 78.1-100 |

Inherent variability of tissue from different patients can result in different cell yield in culture. Therefore, it was not practical to strictly define the timing of cell passages or number and type of culture vessels required at each passage to attain target cell numbers. Typically renal cells undergo 2 or 3 passages; however, duration of culture and cell yield can vary depending on the cell growth rate.

Cells were detached for harvest or passage with TrypLE (Invitrogen). Viability was assessed via Trypan Blue exclusion and enumeration was performed manually using a hemacytometer or using the automated Cellometer® counting system (Nexcelom Bioscience, Lawrence Mass.) or AO/DAPI using a NC-200 nucleocounter.

Cryopreservation of cultured cells. Expanded renal cells may be cryopreserved to accommodate for inherent variability of cell growth from individual patients and to deliver therapeutic, e.g., an enriched heterogeneous renal cell population, if needed, on a pre-determined clinical schedule. Cryopreserved cells also provide a backup source of cells in the event that therapeutic/additional therapeutic doses are needed (e.g., additional doses in the event of delay due to patient sickness, unforeseen process events, etc.). Conditions have been established that have been used to cryopreserve cells and recover viable, functional cells upon thawing.

If expanded renal cells were cryopreserved, cells were suspended to a final concentration of about 50×10⁶ cells/mL in Cryopreservation Solution and dispensed into vials. One ml vials containing about 50×10⁶ cells/mL were placed in the freezing chamber of a controlled rate freezer and frozen at a pre-programmed rate. After freezing, the cells were transferred to a liquid nitrogen freezer for in-process storage.

Preparation of an Enriched Heterogeneous Renal Cell Population. The enriched heterogeneous renal cell population can be prepared from the final culture vessels that have been grown from cryopreserved cells or directly from expansion cultures.

For cryopreserved cells, the cells were thawed and plated on tissue culture vessels for one final expansion step. When the final culture vessels were approximately 50-100% confluent, the cells were ready for processing for enriched heterogeneous renal cell separation. Media exchanges and final washes of NKA diluted any residual Cryopreservation Solution in the final product.

Once the final cell culture vessels reached at least 50% confluence the culture vessels were transferred to a hypoxic incubator set for 2% oxygen in a 5% CO₂ environment at 37° C. and cultured overnight. Cells may be held in the oxygen-controlled incubator set to 2% oxygen for less than 24 hours or overnight. Exposure to the more physiologically relevant low-oxygen (2%) environment improved cell separation efficiency and enabled greater detection of hypoxia-induced markers such as VEGF.

After the cells had been exposed to the hypoxic conditions for a sufficient time (overnight to 48 hours), the cells were detached with TrypLE (Invitrogen). Viability was assessed via Trypan Blue exclusion and enumeration was performed manually using a hemacytometer or using the automated Cellometer® counting system (Nexcelom Bioscience, Lawrence Mass.) or AO/DAPI using the NC-200 nucleocounter. Cells were washed once with OptiMEM and resuspended to about 850×10⁶ cells/mL in DPBS.

Centrifugation across a density boundary/interface was used to separate harvested renal cell populations based on cell buoyant density. Renal cell suspensions were separated by centrifugation over a 7% iodixanol Solution (OptiPrep; 60% (w/v)) in OptiMEM.

The 7% OptiPrep density interface solution was prepared and refractive index indicative of desired density was measured (R.I. 1.3456+/−0.0004) prior to use. Harvested renal cells were layered on top of the solution. The density interface was centrifuged at 800×g for 20 min at room temperature (without brake) in either centrifuge tubes or a cell processor (e.g., COBE 2991). The cellular fraction exhibiting buoyant density greater than approximately 1.045 g/mL was collected after centrifugation as a distinct pellet. Cells maintaining a buoyant density of less than 1.045 g/mL were excluded and discarded.

The enriched heterogeneous renal cell population pellet was re-suspended in DPBS. The carry-over of residual OptiPrep, FBS, culture medium and ancillary materials in the final product was minimized by 4 DPBS wash steps.

### Example 2: Identification, and Determination, of Nephrogenic Marker Expression by Enriched Heterogeneous Renal Cell Populations Indicating their Therapeutic Potential

Introduction. Regeneration of complex tissues and organs, including kidney, may leverage mechanistic elements common to development and organogenesis. Expression of multiple markers typically associated with the earliest signaling events during embryonic nephrogenesis would indicate that an enriched heterogeneous renal cell population, e.g., prepared via the process described in Example 1, has de-differentiated and acquired more renal progenitor-like properties. The introduction of an enriched heterogeneous renal cell population having these traits into diseased renal parenchyma may trigger onset of critical signaling cascades that normally mediate nephrogenesis, but which (in the context of the adult parenchyma) can be interpreted as regeneration.

Method. Enriched heterogeneous renal cell populations, e.g., prepared as described in Example 1, were tested for the expression of nephrogenic markers by FACS analysis. Antibodies used for detection of nephrogenic marker expression of cells are identified in Table 4.

TABLE 4
Antibodies useful for FACS analysis to detect developmental
markers expressed by renal cell populations

| Antigen | Antibody | Conjugate | Catalog # | Vendor |
|---|---|---|---|---|
| Pax2 | Rabbit monoclonal | None | EP3251/ab79389 | Abcam |
| FoxD1 | Rabbit, polyclonal, N-term | None | ab179940 | Abcam |
| Six2 | Monoclonal Six1, H4 | PE | sc377193-PE | Scbt |
| Osr1 | Monoclonal IgGa, C8 | PE | sc376545-PE | Scbt |
| Bmp7 | Rabbit polyclonal, full length | None | ab27569 | Abcam |
| Bim | Rabbit monoclonal, HY36 | AlexaFluor488 | ab200667 | Abcam |
| Lim1 | Rabbit polyclonal | None | NB110-12933 | Novus bio |
| Sal1 | Monoclonal IgG2a | None | ab41974 | Abcam |
| GDNF | Rabbit monoclonal | None | ab176564 | Abcam |
| RET | Rabbit monoclonal | AlexaFluor488 | ab237105 | Abcam |
| Wnt9b | Rabbit polyclonal | None | NBP1-44348 | Novus bio |
| Bmp4 | Rabbit monoclonal | AlexaFluor488 | ab200794 | Abcam |
| Wt1 | Rabbit monoclonal | None | ab89901 | Abcam |
| Wnt4 | Rabbit polyclonal | None | ab91226 | Abcam |
| Pax8 | Monoclonal IgG1 | None | ab53490 | Abcam |
| Fgf8 | Monoclonal, full protein | None | ab89550 | Abcam |
| Lhx1 | Monoclonal IgG2a | PE | sc-515631 PE | scbt |
| Notch1 | Rabbit polyclonal | None | ab118824 | Abcam |
| Muc1 | Rabbit monoclonal | None | ab109185 | Abcam |

Cell preparation for FACS. To perform FACS analysis, cells of the enriched heterogeneous renal cell populations were harvested by trypsin digestion and centrifuged at 400 g for 10 minutes to form a cell pellet. The cell pellet was washed two times with phosphate buffered saline (PBS) and resuspended in a solution of 4% paraformaldehyde with saponin, for cell permeabilization where required for detection of intracellular antigens, for fixation. Following fixation, the cells were pelleted and washed twice with 1×Wash Buffer (Becton Dickenson, proprietary composition) and resuspended in 100 μl to 200 μl Wash Buffer.

FACS staining and analysis. FACS staining was carried out by adding 2 μg of primary antibody (directly labeled or unlabeled), as indicated in Table 4, for 20 minutes at 4° C. Following the incubation of the cells with antibody, the cells were washed twice with Wash Buffer. If needed, e.g., if the primary antibody was unlabeled, the cells were further immunolabeled with an appropriate secondary antibody bearing a detectable fluorophore such as AlexaFluor 488. To permit appropriate gating of channels during FACS analysis, appropriate isotype controls and secondary antibody controls were set up in parallel with the principle staining reactions.

All cell populations were again washed with Wash Buffer and resuspended in 200 μl PBS. Analysis of the immunostained enriched heterogeneous renal cell population was carried out on a MACSQuant Analyzer.

Results. In a total of 18 clinical enriched heterogeneous renal cell population samples, particular levels of expression of 5 nephrogenic markers was observed. See Tables 5 and 6.

TABLE 5
Expression of nephrogenic markers in enriched heterogeneous
renal cell populations with therapeutic potential

| Sample | Six2 | OSR1 | LHx1 | RET | FGF8 |
|---|---|---|---|---|---|
| NKA-013 | 0 | 43.77 | 13.64 | 2.49 | 0 |
| NKA-031 | 0 | 78.66 | 21.48 | 10.15 | 0 |
| NKA-056 | 4.36 | 65.19 | 3.84 | 14.25 | 0.49 |
| NKA-048 | 0.12 | 71.53 | 11.66 | 9.34 | 0.25 |
| NKA-028 | 0.18 | 72.77 | 13.92 | 18.8 | 0.63 |
| NKA-039 | 0 | 84.49 | 5.65 | 6.49 | 1 |
| NKA-030 | 0.18 | 96.28 | 25.14 | 19.1 | 1.39 |
| NKA-023 | 0.58 | 94.37 | 31.78 | 10.86 | 0.54 |
| NKA-032 | 0.99 | 96.72 | 38.33 | 2.07 | 0.23 |
| NKA-070 | 4.08 | 97.19 | 42.58 | 1.33 | 1.08 |
| NKA-087 | 8.11 | 98.29 | 90.79 | 1.98 | 0.31 |
| NKA-089 | 1.7 | 91.95 | 31.46 | 1.29 | 1.15 |
| NKA-042 | 0.24 | 52.82 | 0.87 | 1.42 | 0.01 |
| NKA-034 | 0.98 | 57.23 | 2.53 | 1.26 | 2.65 |
| NKA-092 | 3.11 | 74.95 | 3.97 | 0.58 | 0.11 |
| NKA-063 | 0.32 | 67.2 | 8.68 | 1.45 | 0.16 |
| NKA-097 | 0.06 | 69.29 | 6.43 | 1.09 | 0.08 |
| NKA-075 | 1.12 | 81.8 | 6.63 | 11.37 | 0.52 |

TABLE 6
Summary of range of percent of cells of the enriched
heterogeneous renal cell populations, having therapeutic
potential, expressing the nephrogenic markers

| Marker | Low (% of cells) | High (% of cells) |
|---|---|---|
| SIX2 | 0.04 | 6 |
| OSR1 | 36.3 | 84.43 |
| LHX1 | 8.5 | 57.1 |
| RET | 49.1 | 89.4 |
| FGF8 | 0.48 | 58.55 |

Nephrin was also found to be expressed by about between 4.34 and 98.72 percent of cells of the enriched heterogeneous renal cell population having therapeutic potential.

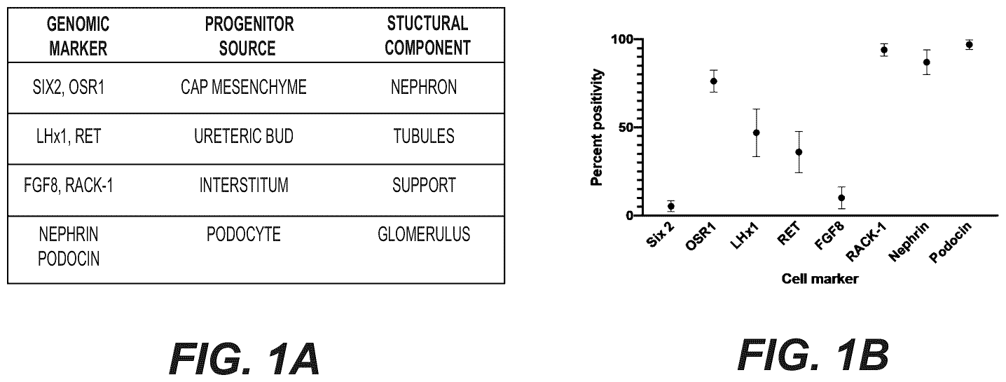

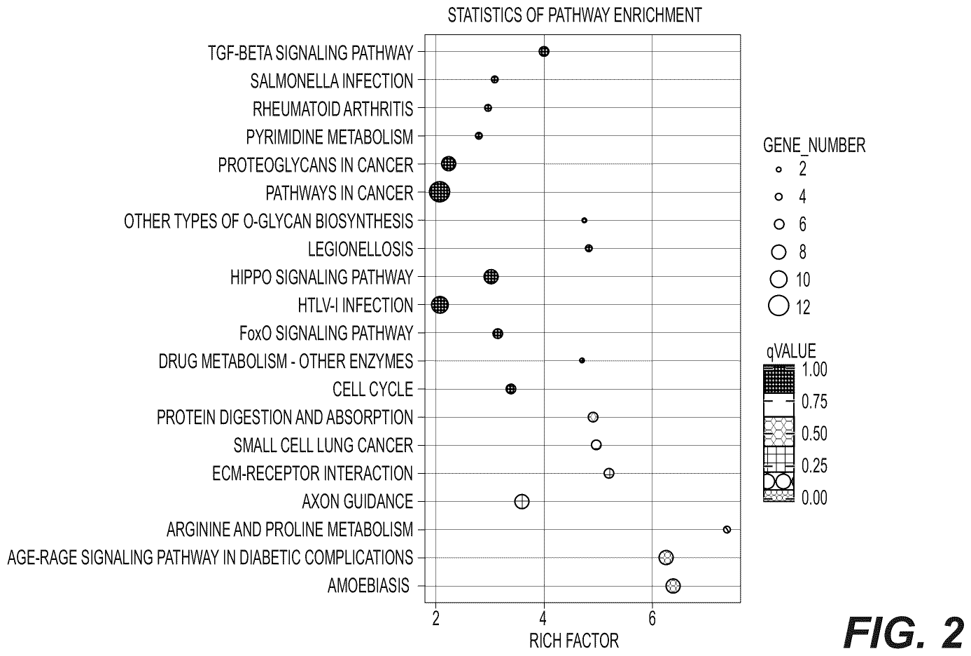

Analysis of further clinical enriched heterogeneous renal cell population samples was conducted, again to determine expression levels of the 5 nephrogenic markers. In addition to the 5 nephrogenic markers, particular levels of nephrin and podocin (which may be important to glomerular development during nephrogenesis) and RACK-1 were also determined. See FIG. 1B.

The regeneration of kidney may need to leverage mechanistic elements common to development and organogenesis. Expression of markers typically associated with the earliest signaling events in embryonic nephrogenesis would indicate that an enriched heterogeneous renal cell population, e.g., prepared as described in Example 1, has dedifferentiated and acquired more renal progenitor-like properties. Therefore, the introduction of such an enriched heterogeneous renal cell population into a diseased kidney may trigger onset of critical signaling cascades that normally mediate nephrogenesis, but which (in the context of the adult parenchyma) can be interpreted as regeneration. Expression of nephrogenic markers SIX2, OSR1, LHX1, RET and FGF8 may explain a mechanism as to how enriched heterogeneous renal cell populations are able to provide therapeutic benefit, e.g., via a regenerative effect to a diseased kidney. Enriched heterogeneous renal cell populations, e.g., prepared consistent with the method described in Example 1, have demonstrated therapeutic effects such as delaying dialysis, decreasing albumin-to-creatinine ratios and increasing eGFR in patients in need of treatment for kidney disease. A brief description of each of the nephrogenic markers and its role in nephrogenesis follows.

SIX2 is a member of the vertebrate gene family which encodes proteins homologous to the Drosophila ‘sine oculis’ homeobox protein. The SIX2 protein is a transcription factor that plays an important role in the development of several organs, including kidney, skull and stomach. During kidney development, SIX2 maintains cap mesenchyme multipotent nephron progenitor cells in an undifferentiated state by opposing the inductive signals emanating from the ureteric bud and cooperates with WNT9B to promote renewing progenitor cells proliferation. SIX2 may function through its interaction with TCF7L2 and OSR1 in a canonical WNT signaling independent manner preventing the transcription of differentiation genes in cap mesenchyme such as WNT4. Additionally, it acts independently of OSR1 to activate expression of many cap mesenchyme genes, including itself, GDNF and OSR1.

OSR1 is the earliest marker of the intermediate mesoderm, which will form the gonads and kidneys. This expression is not essential for the formation of the intermediate mesoderm but rather for the differentiation towards renal and gonadal structures. OSR1 acts upstream of and causes expression of the transcription factors LHX1, PAX2 and WT1 which are involved in early urogenital development. In normal kidney development, activation of the PAX2-EYA1-HOX11 complex and subsequent activation of SIX2 and GDNF expression allows for branching of the ureteric bud and maintenance of the nephron-forming cap mesenchyme. SIX2 maintains the self-renewing state of the cap mesenchyme and GDNF, via the GDNF-RET signalling pathway, is required for attraction and branching of the growing ureteric bud. Within the developing kidney, OSR1 expressing cells will become mesangial cells, pericytes, ureteric smooth muscle and the kidney capsule. The cell types that OSR1 expressing cells will differentiate into are determined by the timing of loss of expression—cells that will become part of the vasculature or ureteric epithelium lose expression of OSR1 early (E8.5), and those that become nephrons lose expression later (E11.5). All three stages of kidney formation are affected in mice lacking OSR1 expression and are similar to mice with reduced WT1 and PAX2 expression—the Wollfian duct is abnormal, there are fewer mesonephric tubules and the kidney-forming metanephros and gonads are missing. In embryonic day 10.5, embryos lacking OSR1 expression fail to grow a ureteric bud that migrates into the uncompacted metanephric mesenchyme. The lack of inductive signals from the ureteric bud combined with a downstream reduction in PAX2 expression results in apoptosis and agenesis of the kidney

LHX1 is a transcription factor. In the vertebrate embryo, the kidney is derived from the intermediate mesoderm. The LIM-class homeobox transcription factor LHX1 is expressed early in the intermediate mesoderm and is one of the first genes to be expressed in the nephric mesenchyme. Depletion of LHX1 in Xenopus embryos results in almost complete loss of the kidney field.

RET, among other things, is necessary for normal kidney development and the production of sperm (spermatogenesis). The RET protein spans the cell membrane, so that one end of the protein remains inside the cell and the other end projects from the outer surface of the cell. This positioning of the protein allows it to interact with specific factors outside the cell and to receive signals that help the cell respond to its environment. When molecules that stimulate growth and development (growth factors) attach to the RET protein, a complex cascade of chemical reactions inside the cell is triggered. These reactions instruct the cell to undergo certain changes, such as dividing or maturing to take on specialized functions.

The FGF8 gene encodes fibroblast growth factor 8 (FGF8). This protein is part of a family of proteins called fibroblast growth factors that are involved in many processes, including cell division, regulation of cell growth and maturation, and development before birth. FGF8 attaches (binds) to another protein called fibroblast growth factor receptor 1 (FGFR1) on the cell surface, which triggers a cascade of chemical reactions inside the cell.

A panel of nephrogenic markers expressed by enriched heterogeneous renal cell populations having therapeutic potential has been identified. Expression of these markers provides evidence that cells of enriched heterogeneous renal cell populations have the ability to influence signaling cascades that mediate nephrogenesis or regeneration of the kidney, and thereby provide a therapeutic effect. It is noted that there is some variability in expression of the markers, which is expected owing to the autologous sourcing of the cells.

### Example 3: Identification of Transcript Modulations of Mechanistic Significance for Therapeutic Bioactivity of Enriched Heterogeneous Renal Cell Populations

Introduction: To further understand mechanisms that underlie reparative, restorative and/or regenerative activities of the enriched heterogeneous renal cell populations, genome-wide transcriptomic profiling was performed on subpopulations of renal cells included therein. Four component enriched renal cell subpopulations (B2, B3, B4 and B5), prepared by density gradient separation of PreG (pre-gradient fractionation) material, are included in enriched heterogeneous renal cell populations. In this Example, transcriptomic profiles of rodent PreG material and the four component enriched renal cell subpopulations (B2, B3, B4 and B5) that make up rodent enriched heterogeneous renal cell populations were separately analyzed, compared and contrasted. By separately analyzing each of the four component enriched renal cell subpopulations (B2, B3, B4 and B5) and comparing their transcriptomic profiles to that of the PreG material, it was possible to understand contributions of each to the enriched heterogeneous renal cell population's observed reparative, restorative and regenerative bioactivity.

Materials and Methods/Preparation of Renal Cell Populations: Kidneys from 3 independent donors (5 week old, male, isogenic Lewis laboratory rats) were used as starting material for 3 independent biological replicates of all renal cells population and subpopulations characterized in this analysis. Preparation of selected bioactive primary renal cells from whole rat kidney has been described in detail (A. T., Guthrie, K. I., Kelley, R. Methods Mol Biol 1001, 53, 2013; Kelley, R., et al. American Journal of Physiology & Renal Physiology 299, 1026, 2010; Kelley, R., et al. Cell Transplantation 22, 1023, 2013). Briefly, whole kidneys were harvested from 5 week old male Lewis rats (Hilltop Labs, Scottsdale, PA, USA) and kidney tissue was dissociated enzymatically in a buffer containing 4.0 units/mL dispase (Stem Cell Technologies, Inc., Vancouver, BC, Canada) and 300 units/mL collagenase IV (Worthington Biochemical, Lakewood, NJ, USA). Red blood cells and debris were removed by centrifugation through 15% iodixanol (Optiprep®, Axis Shield, Norton, MA, USA). Primary renal cells were seeded onto tissue culture treated polystyrene plates (NUNC, Rochester, NY, USA) and cultured in 50:50 media, a 1:1 mixture of high glucose Dulbecco's Modified Eagle Medium (DMEM):Keratinocyte Serum Free Medium (KSFM) containing 5% Fetal Bovine Serum (FBS), 2.5 μg EGF, 25 mg Bovine Pituitary Extract (BPE), 1×ITS (insulin/transferrin/sodium selenite medium supplement), and antibiotic/antimycotic (all from Invitrogen, Carlsbad, CA, USA). Prior to post-culture cell separation, primary renal cell cultures were transferred from atmospheric oxygen conditions (21%) to a more physiologically relevant low-oxygen (2%) environment for 24 hours, to improve cell separation efficiency. Separation of primary renal cell cultures, prepared as 75×10⁶ cells in 2 mL unsupplemented KSFM (uKSFM), was performed by centrifugation through a four-step iodixanol (OptiPrep; 60% w/v in uKSFM) density gradient layered specifically for rodent (16%, 13%, 11%, and 7%) in 15 mL conical polypropylene tubes and centrifuged at 800×g for 20 minutes at room temperature (without brake). After centrifugation, cellular sub-fractions were extracted from the gradient via pipette and collected as 4 distinct bands (B1-B4) and a pellet (B5). All bands were washed 3 times with sterile phosphate buffered saline (PBS) prior to use. A pre-gradient sample (“PreG”) and whole kidney tissue sample (“Macro”) was collected from each rodent for comparative purposes. Culture conditions used for each rodent cell population are summarized in Table 7 below.

TABLE 7
Culture conditions and gradient load¹

| Cell | Seeding | Culture | Final | Gradient |
|---|---|---|---|---|
| Prep | Density | time | Confluency | Load |
| RK086 | 17.5e⁶/flask | 3 d 21% O₂ | 100% | 72.8e⁶ |
| | | 1 d 2% O₂ | | |
| RK087 | 15e⁶/flask | 2 d 21% O₂ | 85% | 91e⁶ |
| | | 1 d 2% O₂ | | |
| RK097 | 19.3e⁶/flask | 2 d 21% O₂ | 85% | 92.5e⁶ |
| | | 1 d 2% O₂ | | |

¹Gradient load refers to the number of PreG cells loaded onto the iodixinol gradient for separation into the component enriched renal cell populations included in the enriched heterogeneous renal cell, e.g., SRC, population.

Materials and Methods/Transcript Analysis: RNA for genome-wide transcript analysis was prepared using Qiagen's RNA isolation kit according to the manufacturer's instructions. 2ug of RNA from each sample normalized as per Table 8 below was used for genome-wide transcript analysis on the Affymetrix GeneChip Rat Genome 230 2.0 Array (Wake Forest University Health Sciences Microarray Core Facility, Winston-Salem, NC).

TABLE 8
RNA concentration and normalization
RNA Normalization

| | | | | | Vol, | Norm |
|---|---|---|---|---|---|---|
| | | Fraction | Symbol | ng/ul | 2 μg | 20 μl |
| 1 | RK086 | 3812 | PreG | 412.19 | 4.852 | 15.148 |
| 2 | | 3813 | B1 | 511.62 | 3.909 | 16.091 |
| 3 | | 3814 | B2 | 460.28 | 4.345 | 15.655 |
| 4 | | 3815 | B3 | 284.08 | 7.040 | 12.960 |
| 5 | | 3816 | B4 | 163.64 | 12.222 | 7.778 |
| 6 | | 3817 | Pellet | 354.38 | 5.644 | 14.356 |
| 7 | RK087 | 3821 | Macro | 213.05 | 9.387 | 10.613 |
| 8 | | 3825 | PreG | 301.08 | 6.643 | 13.357 |
| 9 | | 3826 | B1 | 363.74 | 5.498 | 14.502 |
| 10 | | 3827 | B2 | 351.53 | 5.689 | 14.311 |
| 11 | | 3828 | B3 | 370.35 | 5.400 | 14.600 |
| 12 | | 3829 | B4 | 387.13 | 5.166 | 14.834 |
| 13 | | 3830 | Pellet | 136.67 | 14.634 | 5.366 |
| 14 | RK097 | 4692 | Macro | 125.76 | 15.903 | 4.097 |
| 15 | | 4697 | PreG | 379.67 | 5.268 | 14.732 |
| 16 | | 4698 | B1 | 366.56 | 5.456 | 14.544 |
| 17 | | 4699 | B2 | 420.82 | 4.753 | 15.247 |
| 18 | | 4700 | B3 | 439.3 | 4.553 | 15.447 |
| 19 | | 4701 | B4 | 350.43 | 5.707 | 14.293 |
| 20 | | 4702 | Pellet | 167.94 | 11.909 | 8.091 |

RNA was normalized by resuspending a total of 2 ug of RNA in a total volume of 20 ul. In Table 2, the column “vol, 2 ug” lists the volume of the RNA preparation representing a total of 2 ug of RNA. The column “Norm 20 ul” lists the volume of additional buffer to be added to make up a final volume of 20 ul.

Materials and Methods/Data Analysis: Affymetrix GeneChip data was normalized using RMA (Rafael et al., 2003. Nucleic Acids Research 31:e15). The gene expression profile from each component enriched renal cell subpopulation (B1, B2, B3, B4, B5) was compared to the gene expression profile in the PreG material using paired t-test. Differentially expressed genes were detected by paired t-test at P<0.05, and were then analyzed by DAVID (http://david. abcc.ncifcrf.gov) to detect Gene Ontology (GO) categories and Kyoto Encyclopedia of Genes and Genomes (KEGG) Pathways that were significantly different between each fraction and PreG. For GO analysis, GO-BP-FAT was used, which is GO biological process categories provided by DAVID to minimize redundancy and increase specificity in GO terms. Bonferroni adjustment was applied to P-values generated from GO and KEGG pathway analysis for multiple testing correction. KEGG pathway plots were generated using Pathview in Bioconductor (Weijun Luo and Cory Brouwer. Bioinformatics, 29(14):1830-1831, 2013). Gene networks for GO categories were generated using QIAGEN's Ingenuity Pathway Analysis (IPA®, QIAGEN Redwood City, www.qiagen.com/ingenuity).

Results/Subfraction B1: GO analysis of the B1 subpopulation relative to the PreG material showed significant down-regulation of GO categories associated with regulation of the cell cycle (P=9.40E-04), cell division (P=9.81E-05) and cell cycle phase (P=0.016) (Table 9). Similar outcomes were also generated by KEGG pathway analysis (Table 9). Down-regulated cycle pathway genes were distributed in each phase of the cell cycle, including cell growth (G1, G2), DNA replication (S) and Mitosis (M). For example, key cell cycle regulatory proteins subject to negative regulation in B1 relative to PreG included CDK2, CDK7, CYCE, CDC45, ORC, CHK1, CHK2, MAD2, CDC20 and APC. In addition, GO/KEGG categories related to regulation of transcription, including regulation of transcription (P=0.001), regulation of RNA metabolic process (P=0.044) and spliceosome (P=0.002) were significantly down-regulated (Table 9).

TABLE 9
GO categories and KEGG pathways that were significantly
up or down regulated in B1 by comparing to PreG.

| | | | | Bonferroni |
|---|---|---|---|---|
| | Category | Term | Raw_P | adjusted P |
| Up- | GO_BP_FAT | GO-0019221: cytokine-mediated | 7.12E−06 | 0.022732 |
| regulated | | signaling pathway | | |
| | | GO-0022610: biological adhesion | 1.42E−05 | 0.044785 |
| | | GO-0007155: cell adhesion | 1.42E−05 | 0.044785 |
| | KEGG | rno04142: Lysosome | 2.57E−06 | 4.44E−04 |
| Down- | GO_BP_FAT | GO-0051301: cell division | 3.24E−08 | 9.81E−05 |
| regulated | | GO-0007049: cell cycle | 3.11E−07 | 9.40E−04 |
| | | GO-0045449: regulation of | 4.95E−07 | 0.001496 |
| | | transcription | | |
| | | GO-0022403: cell cycle phase | 5.32E−06 | 0.015942 |
| | | GO-0051252: regulation of RNA | 1.51E−05 | 0.04449 |
| | | metabolic process | | |
| | KEGG | rno03040: Spliceosome | 1.35E−05 | 0.00234 |
| | | rno04110: Cell cycle | 2.45E−04 | 0.041792 |

These transcriptomic data suggested that B1 subpopulations present significantly less proliferative and regenerative potential, consistent with observed absence of functional impact by B1 cells on renal pathophysiology in rodent models of CKD (Bruce, A. T., Guthrie, K. I., Kelley, R. Methods Mol Biol 1001, 53, 2013; Kelley, R., et al. American Journal of Physiology & Renal Physiology 299, 1026, 2010; Kelley, R., et al. Cell Transplantation 22, 1023, 2013).

Significantly up-regulated GO/KEGG categories in the B1 subpopulation relative to the PreG material included cytokine-mediated signaling pathway (P=0.023), which is involved in immune response, and lysosome (P=4.44E-04) which is involved in the digestion of foreign material and cell waste. Cell adhesion (P=0.045) was also up-regulated (Table 9). These data, again, are consistent with the observed absence of functional impact by B1 cells on renal pathophysiology in rodent models of CKD. The B1 enriched renal cell subpopulation is not a component of the enriched heterogeneous renal cell populations.

Results/Subfraction B2: The transcriptomic profile of B2 was found to be similar to PreG at the pathway level. No GO/KEGG categories were identified as significantly up-regulated. Only one pathway, ECM-receptor interaction (rno04512), was found to be significantly down-regulated (P=0.039) relative to the PreG material. Consistent with the ECM-receptor interaction down-regulation, numerous ECM proteins, including collagen, laminin, reelin, tenascin, vitronectin and thrombospondin (THBS), were significantly down-regulated in B2; it is well known that excessive deposition of extracellular matrix (ECM) components causes tissue fibrosis (Liu Y. Kidney International 69, 213, 2006; Kisseleva and Brenner, 2008. Proc Am Thorac Soc 5: 338-42; El-Nahas, 2003. Kidney Int 64: 1553-63). In addition, renal fibrosis is the hallmark of disease progression from chronic kidney disease (CDK) to end-stage kidney disease.

In contrast to the negatively regulated transcriptomic profile observed with ECM-associated genes, Receptor for Hyaluronan Mediated Motility (RHAMM; also known as CD168 or Hyaluronan-mediated motility receptor (HMMR)) expression was specifically up-regulated in the B2 subpopulation relative to the PreG material (fold change=1.114, P=0.030). The expression of RHAMM has been directly linked to regenerative bioactivity in a number of in vitro and in vivo model systems. For example, RHAMM has also been shown to promote each of angiogenesis, regeneration of 3D salivary gland spheroids, and Xenopus tadpole tail and cell motility. Thus, the increased expression level of RHAMM could be beneficial for tissue regeneration (Pradhan-Bhatt et al., 2014. Laryngoscope 124: 456-461; Contreras et al., 2009. Development 136: 2987-96; Tolg et al., 2006. J Cell Biol 175: 1017-28; Savani R C, et al. 2001. J. Biol. Chem. 276 (39): 36770-8; Hall C L, et al. 1994. J. Cell Biol. 126 (2): 575-88; Hamilton S R, et al. 2007. J. Biol. Chem. 282 (22): 16667-80). Further, hyaluronic acid (HA), for which RHAMM is a receptor, is an ECM component shown to: be present throughout the interstitial space of the developing kidney (Bakala, H., et al., 1988. J Morphol 196: 1-14); stimulate ureteric bud differentiation to collecting ducts; and mesenchymal to epithelial transition of the metanephric mesenchyme in a molecular weight independent manner. RHAMM's significant up-regulation in the B2 subpopulation and these data raise interesting possibilities for renal tissue engineering and cell-based medicine. By way of example, scaffolds designed to release HA of defined molecular weights at certain concentrations may be implanted within kidney with the expectation of modulating regenerative outcomes.

Results/Subfraction B3: KEGG pathway analysis found categories related to complement and coagulation cascades are significantly up-regulated in the B3 subpopulation relative to the PreG material (P=0.008) (Table 10). Up-regulated genes included complement component genes, such as C2, C3 and C4, and coagulation related genes, such as fibrinogen (FG) and coagulation factor XIII (F13). The complement and coagulation system is considered the “first line of defense” against injuries and invaders (Choi G, et al. Swiss Med Wkly. 2006; 136:139-144). Thus, the up-regulation of this pathway indicates an increased immune response. Consistent with KEGG analysis, GO analysis also found a significant increase in the humoral immune response (P=0.025) (Table 10). It is well established that multiple components of both cellular and humoral immune systems contribute to reparative, restorative, or regenerative outcomes in multiple organs and tissues, including limbs, skeletal muscle, heart and nervous system (Aurora and Olson, 2014. Cell Stem Cell 15: 14-25).

TABLE 10
GO categories and KEGG pathways that were significantly
up- or down-regulated on B3 by comparing to PreG

| | | | | Bonferroni |
|---|---|---|---|---|
| | Category | Term | Raw_P | adjusted P |
| Up- | GO_BP_FAT | GO: 0043112~receptor metabolic | 2.14E−06 | 0.005782 |
| regulated | | process | | |
| | | GO: 0007242~intracellular | 3.59E−06 | 0.009661 |
| | | signaling cascade | | |
| | | GO: 0006959~humoral immune | 9.21E−06 | 0.024598 |
| | | response | | |
| | | GO: 0007167~enzyme linked | 1.89E−05 | 0.049871 |
| | | receptor protein signaling | | |
| | | pathway | | |
| | KEGG | rno04610: Complement and | 6.60E−05 | 0.008351 |
| | | coagulation cascades | | |
| Down- | GO_BP_FAT | GO: 0008064~regulation of actin | 2.42E−06 | 0.00522 |
| regulated | | polymerization or depolymerization | | |
| | | GO: 0030832~regulation of actin | 2.42E−06 | 0.00522 |
| | | filament length | | |
| | | GO: 0030833~regulation of actin | 8.68E−06 | 0.018633 |
| | | filament polymerization | | |

Significantly down-regulated GO categories in the B3 subpopulation were related to the regulation of actin polymerization and actin filament length (Table 10). The dynamic reorganization of the actin cytoskeleton is a principal driver of cell migration. Inhibition of actin polymerization may have an adverse effect on cell migration and the therapeutic efficacy of the B3 subpopulation.

Results/Subfraction B4: The B4 subpopulation has been observed to have functional outcomes in rodent models for CKD. Consistent with its observed in vivo therapeutic bioactivity, multiple regeneration-related GO categories, including vasculature development (P=7.76E-07), blood vessel development (P=1.36E-06), blood vessel morphogenesis (P=1.76E-06), angiogenesis (P=1.82E-06) and response to wounding (P=1.35E-05) were found to be significantly up-regulated in the B4 subpopulation over the PreG material (Table 11). The GO category angiogenesis is central to reparative, restorative, or regeneration, in the kidney, heart and multiple other organs and tissues (Takiya and Borojevic, 2011. Kidney Int Suppl 1: 99-102; Kramann and Humphreys, 2014. Semin Nephrol 34: 374-83; Park and Gerecht, 2014. Development 141: 2760-9; Kaully et al., 2009. Tissue Eng Part B 15: 159-69). Key receptors central to angiogenesis and other regeneration-associated signaling pathways, including CXCR4, TEK, FGFR1 and KDR, were found to be up-regulated. Of these, the SDF1/CXCR4 axis is central to renal reparative, restorative, or regeneration in response to ectopic cell-based therapies (Togel and Westenfelder, 2011. Kidney Int Suppl 1: 87-89, Sharma et al., 2011. Stem Cells Dev 20: 933-46; Sagrinati, C., et al. Trends Mol Med 14, 277, 2008). In the GO category response to wounding, many genes that were up-regulated, such as Notch1, Notch3, Timp3, Vwf, Adam15, Gas6, Igfbp1 and Tm4sf4, also belong to the GO categories wound healing and tissue restoration, repair or regeneration. The significant up-regulation of these genes over PreG is also consistent with the regenerative bioactivity of subpopulation B4.

TABLE 11
GO categories significantly up-regulated in B4 compared to PreG

| | | Bonferroni |
|---|---|---|
| GO-BP-FAT Term | Raw P | adjusted P |
| GO: 0001944~vasculature development | 2.61E−10 | 7.76E−07 |
| GO: 0001568~blood vessel development | 4.59E−10 | 1.36E−06 |
| GO: 0048514~blood vessel morphogenesis | 5.91E−10 | 1.76E−06 |
| GO: 0001525~angiogenesis | 6.13E−10 | 1.82E−06 |
| GO: 0009611~response to wounding | 4.55E−09 | 1.35E−05 |
| GO: 0007155~cell adhesion | 9.76E−09 | 2.90E−05 |
| GO: 0022610~biological adhesion | 9.76E−09 | 2.90E−05 |
| GO: 0007242~intracellular signaling | 6.29E−07 | 0.001869 |
| cascade | | |
| GO: 0019932~second-messenger-mediated | 1.40E−06 | 0.004152 |
| signaling | | |
| GO: 0010033~response to organic | 1.41E−06 | 0.004182 |
| substance | | |
| GO: 0055066~di-, tri-valent inorganic | 1.13E−05 | 0.033116 |
| cation homeostasis | | |

Additionally, cell adhesion related GO categories were found to be significantly up-regulated in B4 relative to PreG material (Table 11). Cell adhesion is essential for the incorporation and functional integration of transplanted cells to the host environment. The induction of cell adhesion genes could be beneficial for the homing of transplanted cells.

No analyzed pathways were found to be significantly down regulated in B4.

Results/Subfraction B5: Sub-population B5 was found to be the most distinctive fraction relative to the PreG material at the gene expression level. Forty GO/KEGG categories were significantly different from PreG (Tables 12 and 13).

TABLE 12
GO Categories and KEGG pathways significantly up-regulated in B5 compared to PreG

| | | | Bonferroni |
|---|---|---|---|
| Category | Term | PValue | adjusted P |
| GOTERM_BP_FAT | GO: 0022610~biological adhesion | 1.36E−14 | 4.62E−11 |
| | GO: 0007155~cell adhesion | 1.36E−14 | 4.62E−11 |
| | GO: 0035295~tube development | 7.52E−10 | 2.57E−06 |
| | GO: 0001525~angiogenesis | 2.88E−09 | 9.84E−06 |
| | GO: 0001568~blood vessel development | 9.91E−09 | 3.38E−05 |
| | GO: 0048754~branching morphogenesis of a tube | 1.46E−08 | 5.00E−05 |
| | GO: 0048514~blood vessel morphogenesis | 1.57E−08 | 5.37E−05 |
| | GO: 0001944~vasculature development | 2.29E−08 | 7.82E−05 |
| | GO: 0032989~cellular component morphogenesis | 3.12E−08 | 1.06E−04 |
| | GO: 0000902~cell morphogenesis | 6.13E−08 | 2.09E−04 |
| | GO: 0000904~cell morphogenesis involved in | 1.24E−07 | 4.24E−04 |
| | differentiation | | |
| | GO: 0035239~tube morphogenesis | 1.31E−07 | 4.46E−04 |
| | GO: 0030030~cell projection organization | 1.49E−07 | 5.07E−04 |
| | GO: 0048858~cell projection morphogenesis | 2.42E−07 | 8.25E−04 |
| | GO: 0016337~cell-cell adhesion | 2.79E−07 | 9.52E−04 |
| | GO: 0048812~neuron projection morphogenesis | 5.46E−07 | 0.001862 |
| | GO: 0032990~cell part morphogenesis | 6.75E−07 | 0.0023 |
| | GO: 0048666~neuron development | 7.48E−07 | 0.002549 |
| | GO: 0033273~response to vitamin | 7.72E−07 | 0.002632 |
| | GO: 0001763~morphogenesis of a branching | 1.06E−06 | 0.003597 |
| | structure | | |
| | GO: 0021700~developmental maturation | 1.20E−06 | 0.004074 |
| | GO: 0031175~neuron projection development | 2.56E−06 | 0.008704 |
| | GO: 0050678~regulation of epithelial cell | 6.75E−06 | 0.022778 |
| | proliferation | | |
| | GO: 0007584~response to nutrient | 1.49E−05 | 0.049576 |
| KEGG | rno04514: Cell adhesion molecules (CAMs) | 5.89E−05 | 0.009201 |

TABLE 13
GO categories and KEGG pathways significantly
down-regulated in B5 compared to PreG

| | | | Bonferroni |
|---|---|---|---|
| Category | Term | Raw-P | adjusted-P |
| GOTERM_BP_FAT | GO: 0044265~cellular macromolecule catabolic | 5.44E−08 | 1.79E−04 |
| | process | | |
| | GO: 0007242~intracellular signaling cascade | 2.07E−07 | 6.83E−04 |
| | GO: 0051603~proteolysis involved in cellular | 5.90E−07 | 0.001943 |
| | protein catabolic process | | |
| | GO: 0044257~cellular protein catabolic process | 7.90E−07 | 0.002602 |
| | GO: 0043632~modification-dependent | 1.48E−06 | 0.004857 |
| | macromolecule catabolic process | | |
| | GO: 0019941~modification-dependent protein | 1.48E−06 | 0.004857 |
| | catabolic process | | |
| | GO: 0015031~protein transport | 1.99E−06 | 0.006541 |
| | GO: 0030163~protein catabolic process | 2.23E−06 | 0.007313 |
| | GO: 0045184~establishment of protein | 2.78E−06 | 0.009125 |
| | localization | | |
| | GO: 0009057~macromolecule catabolic process | 3.47E−06 | 0.011365 |
| KEGG | rno04120: Ubiquitin mediated proteolysis | 1.17E−07 | 2.02E−05 |
| | rno04310: Wnt signaling pathway | 8.10E−06 | 0.001392 |
| | rno05200: Pathways in cancer | 8.19E−06 | 0.001408 |
| | rno05222: Small cell lung cancer | 2.02E−05 | 0.003468 |
| | rno04530: Tight junction | 2.11E−04 | 0.035682 |

Up-regulated GO/KEGG categories included cell adhesion, angiogenesis, branching morphogenesis of a tube, cell morphogenesis, regulation of epithelial cell proliferation, blood vessel development, blood vessel morphogenesis, vasculature development, cellular component morphogenesis, cell morphogenesis, cell morphogenesis involved in differentiation, tube morphogenesis, cell projection organization and morphogenesis, cell part morphogenesis, morphogenesis of a branching structure, developmental maturation and response to nutrient, etc. The majority of these significantly up-regulated GO/KEGG categories are clearly involved in catalyzing reparative, restorative, or regenerative outcomes.

As observed with B4, the key pro-angiogenic receptors CXCR4, TEK and KDR are prominently up-regulated in B5. In addition, the pro-angiogenic ligands PGF, ANGPT2, ANGPT4, VEGFA and PDGFA were observed to be significantly up-regulated in B5 subpopulation relative to the PreG material. The regenerative bioactivity of these ligands is well established.

Several significantly differentially regulated pathways of particular interest, included the canonical Wnt/b-catenin signaling pathway. The canonical Wnt/b-catenin signaling pathway was significantly down-regulated in the B5 subpopulation relative to the PreG material (Table 13). Wnt signaling is intimately involved in nephrogenesis and organogenesis but may also in the appropriate context be pro-fibrotic and contribute to the maintenance of the disease state (Kawakami et al., 2013. J Pathol 229: 221-31; Shkreli et al., 2011. Nat Med 18: 111-9; Cisternas et al., 2014. Curr Mol Med 14: 510-22). It is possible that the relevant Wnt signaling bioactivity for B5 in the context of SRC may be its pro-fibrotic action; therefore, any reduction in Wnt signaling would presumably be a desired outcome upon implantation within a fibrotic, diseased kidney (Cuevas et al., 2015. Biomed Res Int. Article ID 726012).

Also of interest, the GO category “branching morphogenesis of a tube” was up-regulated in the B5 subpopulation relative to the PreG material. This was of interest given that mammalian nephrogenesis is driven by an iterative process of branching morphogenesis between the ureteric bud, a derivative of the nephric duct, and the surrounding metanephric mesenchyme. A continued branching morphogenesis from the ureteric bud epithelia in response to signaling from neighboring metanephric mesenchyme in turn leads to induction of new aggregates of metanephric mesenchyme at the ureteric bud tips and continued nephrogenic events. This iterative process continues along the radial axis of the developing kidney with the youngest nephrons induced toward the periphery (reviewed by Basu and Ludlow, 2012. Birth Defects Research Part C 96: 30-38). To this end, implantation of an enriched heterogeneous renal cell population, e.g., SRC, in adult rodent kidney has been associated with the induction of neo-nephron-like structures de novo (Basu, J., et al. Cell Transplantation 20, 1771, 2011).

Finally of interest, activation of the regulation of GO category epithelial cell proliferation through the TGF-β1 signaling pathway was noted as consistent with a potential role for enriched heterogeneous renal cell populations, e.g., SRCs, in the promotion of host tubular epithelial cell proliferation. Indeed, to this end, transplanted enriched heterogeneous renal cell populations have been observed to promote tubular cell proliferation in the 5/6Nₓ model. Enriched heterogeneous renal cell population, e.g., SRC, treatment specifically increased the number of Ki67+ proliferating cells specifically in the tubular epithelia. This compartment-specific proliferation may be a direct indicator of therapeutic outcome: epithelial proliferation leads to replenishment of renal function, while interstitial proliferation leads to fibrosis.

A significant down-regulation of multiple GO/KEGG categories associated with protein metabolism in the B5 subpopulation relative to the PreG material was observed and may simply indicate a decreased overall metabolic rate for the B5 subpopulation relative to the PreG material.

Summary Enriched heterogeneous renal cell populations, e.g., SRCs, are manufactured through iodixinol gradient centrifugation of PreG material. Iodixinol gradient centrifugation of the PreG material results in separation of the PreG material into five, B1-B5, subpopulations. Transcriptomic profiling of the B1-B5 subpopulations identified key transcriptomic networks and concomitant signaling pathways that may underlie enriched heterogeneous renal cell populations, e.g., SRCs, mechanisms of action, as manifested by their reparative, restorative and regenerative bioactivity in treatment of chronic kidney disease. Significant differences in the B2-B5 subpopulations included in the final, enriched heterogeneous renal cell population product, relative to the PreG material include: down-regulation of GO/KEGG categories associated with ECM-receptor interaction in subpopulation B2; up-regulation of GO/KEGG categories associated with immune response and down-regulation GO/KEGG categories associated with regulation of actin polymerization in subpopulation B3; up-regulation of GO/KEGG categories associated with reparative, restorative, regeneration and cell adhesion in B4; and forty differentially regulated GO/KEGG categories, including multiple categories unambiguously associated with reparative, restorative or regenerative activity in B5. Subpopulation B1, which is not included in the enriched heterogeneous renal cell population, relative to the PreG material, exhibited significant down-regulation in GO/KEGG categories associated with cell cycle and transcriptional control and significant up-regulation in GO/KEGG categories associated with inflammation.

Taken together, the transcriptomic data suggest that enriched heterogeneous renal cell populations for therapeutic use, e.g., SRCs, catalyze reparative, restorative, or regenerative outcomes in the kidney in part by activating multiple transcriptomic networks associated with the promotion of regeneration while simultaneously suppressing alternate transcriptional pathways that may promote the continued development of renal disease and pathophysiology.

### Example 4: Identification of Developmental Pathways of Mechanistic Significance for the Regenerative Bioactivity of Enriched Heterogeneous Human Renal Cell Populations Used to Treat Chronic Kidney Disease

Introduction: Further building on the rodent data in Example 3, transcriptomic and epigenomic analyses were performed on human enriched heterogeneous renal cell populations prepared for use in clinical trials. Renal Autologous Cell Therapy (REACT) is a novel regenerative medicine candidate for treatment of chronic kidney disease, diabetic nephropathy and congenital anomalies of the kidney and urinary tract (Stavas 2021. Protocol and Baseline Data on Renal Autologous Cell Therapy Injection in Adults with Chronic Kidney Disease Secondary to Congenital Anomalies of the Kidney and Urinary Tract. Blood Purif 50(4-5):678-683; Stenvinkel P, Wadstr\ddot{p}m J, Bertram T, Detwiler R, Gerber D, Brismar T B, et al. Implantation of autologous selected renal cells in diabetic chronic kidney disease stages 3 and 4-clinical experience of a “First in Human” study. Kidney Int Rep. 2016 1(3):105-13). REACT is formulated with an enriched heterogeneous renal cell population (referred to in this Example as SRCs or an SRC population), prepared, or manufactured, from an initial cell population derived from a subject's own renal biopsy (initial cell population referred to in this Example as BRC0s or a BRC0 population). A comparison of the transcriptomic and epigenomic profiles of the human SRC versus BRC0 populations was performed to confirm and further explore mechanisms underlying the reparative and restorative activities of human SRCs being used for treatment of kidney disease.

Materials and Methods/Preparation of SRCs for Use in Treatment of Chronic Kidney Disease: Methodologies for the preparation of selected renal cells from renal tissue biopsies have been described previously in detail (Bruce, A. T., Guthrie, K. I., Kelley, R. Ex vivo culture and separation of functional renal cells. Methods Mol Biol 1001, 53, 2013; Halberstadt et al., 2013, Methods Mol. Biol.).

Materials and Methods/cDNA Library Construction: For library construction, RNA was extracted from each cell population (n=6). After DNase I treatment and performing quality control (QC), 200 ng of high-quality total RNA was used for library construction. Magnetic beads with Oligo (dT) were used to isolate mRNA. The mRNA was fragmented randomly by adding fragmentation buffer, then cDNA was synthesized by using mRNA template and random hexamer primer after adding dNTPs, RNase H and DNA polymerase I. Short fragments were purified and resolved with EB buffer for end repair and single nucleotide A (adenine) addition. After that, the short fragments were connected with sequencing adapters. The double-stranded cDNA library was completed through size selection and PCR enrichment. During the QC steps, Agilent 2100 Bioanaylzer and ABI StepOnePlus Real-Time PCR System were used in the quantification and qualification of the sample library. Lastly, the qualified RNA-seq libraries were sequenced using Illumina NovaSeq6000 by CD Genomics (Shirley, NY) after pooling according to effective concentration and expected data volume.

Materials and Methods/Bioinformatics analysis: The FastQC tool (http://www.bioinformatics.babraham.ac.uk/projects/fastq) was used to perform basic statistics on the quality of the raw reads. Sequencing adapters and low quality data were removed by Trimmomatic (version 0.36) (Juhling, F., et al., metilene: fast and sensitive calling of differentially methylated regions from bisulfite sequencing data. Genome Res, 2016. 26(2): p. 256-62). The filtered clean reads were mapped to the reference genome by HISAT2 (Kanehisa, M., et al., KEGG for linking genomes to life and the environment. Nucleic Acids Res, 2008. 36(Database issue): p. D480-4), and the position and gene characteristics acquired. The Cuffquant and Cuffnorm components of Cufflinks software were used to quantify transcripts and gene expression levels using mapped reads positional information on the gene. The genes numbers at different expression levels as well as the gene expression level of each single gene were analyzed. DESeq was used to analyze the DEG for samples with biological replicates and EBSeq for the samples without replicates. During the analysis, samples were firstly grouped so that comparison between every two groups as a control-treatment pairwise could be done later. During the process, Fold Change≥2 and FDR<0.01 were set as screening criteria. Gene ontology (GO—Gene Ontology Consortium, 2000) enrichment analysis is the set of internationally standardized classification system of gene functional description that attempts to identify GO terms that are significantly associated with the differentially expressed protein coding genes. GO molecules are divided into three main categories: 1) Cellular Component: used to describe the subcellular structure, location and macromolecular complexes, such as nucleoli, telomere and recognition of the initial complex; 2) Molecular Function: used to describe the gene, gene products, individual functions, such as carbohydrate binding or ATP hydrolase activity; and 3) Biological Process: used to describe the products encoded by genes involved in biological processes, such as mitosis or purine metabolism. KEGG Pathway was used to identify the pathways that were significantly enriched in a particular gene compared to the entire genome background. The formula for this analysis was:

$$p = 1 - {\sum }_{i = 0}^{m - 1} \frac{( \begin{matrix}M \\ i\end{matrix} ) ( \begin{matrix}N - M \\ n - i\end{matrix} )}{( \begin{matrix}N \\ n\end{matrix} )} ,$$

where ‘N’ was number of all the genes that have pathway annotation; ‘n’ was the candidate gene number within N; and ‘m’ was the number of gene that annotated with particular pathway. The pathways with FDR≤0.05 were defined as the significant enriched pathways. KOBAS (2.0) was used to perform pathway enrichment analysis.

Materials and Methods/Reduced Representation Bisulfite Sequencing (RRBS): Genomic DNA was isolated from cell samples (BRC0, SRC, n=4) using Nucleon™ BACC Genomic DNA Extraction Kit (GE Healthcare, Life Sciences) and double stranded DNA content quantified using the Qubit High Sensitivity assay (Life Technologies). 1 μg of genomic DNA from each cell sample was digested with MspI (NEB), followed by end preparation and adaptor ligation using Premium RRBS kit (Diagenode). Size selection was performed using AMPure XP beads (Beckman Coulter, Inc.) to obtain DNA fractions of MspI-digested products enriching for the most CpG-rich regions in the range of 150-350 bp. Subsequently, bisulfite treatment was conducted using the ZYMO EZ DNA Methylation-Gold Kit. The converted DNAs were then amplified by twelve cycles of PCR, using 25 μl KAPA HiFi HotStart Uracil+ ReadyMix (2×) and 8-bp index primers with a final concentration of 1 μM each and cleaned-up using AMPure XP beads. The constructed RRBS libraries were sent to CD Genomics (Shirley, NY) for sequencing and bioinformatics analysis. Briefly, the libraries were quantified by a Qubit fluorometer with Quant-iT dsDNA HS Assay Kit (Invitrogen), and sequenced on Illumina Hiseq platform using paired-end 150 bp strategy.

Materials and Methods/RRBS Data Analysis: The FastQC tool (www.bioinformatics.babraham.ac.uk/projects/fastq) was used to perform basic statistics on the quality of the raw reads. Then, sequencing adapters and low quality data of the sequencing data were removed by Trimmomatic (version 0.36) (Juhling, F., et al., metilene: fast and sensitive calling of differentially methylated regions from bisulfite sequencing data. Genome Res, 2016. 26(2): p. 256-62). The BSMAP software was used to map the bisulfite sequence to the reference genome with parameters ‘−n 0 −g 0 −v 0.08 −m 50 −x 1000 (Kanehisa, M., et al., KEGG for linking genomes to life and the environment. Nucleic Acids Res, 2008. 36(Database issue): p. D480-4). The statistical information of the alignment was collected, with only the unique mapped reads being kept for the following analysis. Only methylated cytosines with sequence depth coverage of at least 5 were used. If the base on the alignment was C, methylation occurred; conversely, if the base on the alignment was T, no methylation occurred. The methylation levels of individual cytosines were calculated as the ratio of the sequenced depth of the ascertained methylated CpG cytosines to the total sequenced depth of individual CpG cytosines, i.e., ML=mC/(mC+umC), where ML was the methylation level, mC and umC represented the number of reads supporting methylation C and the number of reads supporting unmethylated C, respectively. The software metilene (version 0.2-7) was used to identify DMR (differentially methylated regions) by a binary segmentation algorithm combined with a two-dimensional statistical test (parameters: −M 300 −m 5 −d 0.1 −t 1 −f 1 −v 0.7) (Bruce et al., 2013. Ex vivo culture and separation of functional renal cells. Methods Mol Biol 1001:53-640. Gene Ontology (referred to as GO, http://www.geneontology.org/) enrichment analysis of DMR-related genes was applied to uncover biological processes of interest. Pathways with a Q value≤0.05 were deemed significantly enriched with DMR-related genes. Based on the results of the DMR annotation and the database of KEGG (Sha et al., 2021. Genome-wide transcriptional profiling of selected renal cells (SRC): identification of functional pathways for regenerative bioactivity in treatment of chronic kidney disease. Manuscript submitted) functional enrichment analysis was performed on genes whose gene body and upstream and downstream regions (upstream 2 k, gene body, and downstream 2 k) were observed to overlap with DMR.

Results/Differentially Expressed Transcripts that Distinguish Between Cells of SRC and BRC0 Populations

A total of n=6 individual donors were used to prepare total RNA and cDNA for library construction and RNA-seq analysis as described in Materials and Methods. A total of 221 DEG were identified that discriminate SRC populations against BRC0 populations. These 221 DEG are composed of 119 genes specifically up-regulated in SRC relative to BRC0 and 102 genes specifically down-regulated in SRC relative to BRC0. A list of the top 20 up-regulated and down-regulated genes in SRC relative to BRC0 is shown in Tables 14 and 15, respectively. The false discovery rate (FDR) for these transcripts was 10⁻⁶ and smaller, reflecting a very high degree of statistical significance.

TABLE 14
Top 20 up-regulated genes in SRC relative to BRC0

| | |
|---|---|
| CDKN2A | cyclin dependent kinase inhibitor 2A |
| IL11 | interleukin-11 |
| FAM101A | refilin-A |
| FN1 | fibronectin 1 |
| C10orf99 | chromosome 10, ORF 99 |
| RUNX2 | runt related transcription factor 2 |
| HHIPL2 | hedgehog interacting protein like 2 |
| CRISPLD2 | cysteine rich secretory protein LCCL domain |
| | containing 2 |
| PREX2 | phosphatidylinositol-3,4,5-trisphosphate dependent |
| | Rac exchange factor 2 |
| ADAMTS6 | ADAM metallopeptidase with thrombospondin type 1 |
| | motif 6 |
| HS3ST3A1 | heparan sulfate-glucosamine 3-sulfotransferase 3A1 |
| COL11A1 | collagen type XI alpha 1 chain |
| LFNG | lunatic fringe (LFNG O-fucosylpeptide 3-beta-N- |
| | acetylglucosaminyltransferase) |
| CGB | chorionic gonadotropin |
| LOX | lysyl oxidase |
| TGFB2 | transforming growth factor beta 2 |
| CXCL3 | CXC motif chemokine ligand 3 |
| BDNF | brain derived neurotrophic factor |
| MOXD1 | monooxygenase DBH like 1 |
| NEFM | neurofilament medium |

TABLE 15
Top 20 down-regulated genes in SRC relative to BRC0

| | |
|---|---|
| CLDN3 | claudin 3 |
| RAI2 | retinoic acid induced 2 |
| UPP1 | uridine phosphorylase 1 |
| PHYHIPL | phytanoyl-CoA 2-hydroxylase interacting protein like |
| PI3 | peptidase inhibitor 3 |
| GYLTL1B | LARGE xylosyl- and glucuronyltransferase 2 |
| MAN1C1 | mannosidase alpha class 1C member 1 |
| AQP1 | aquaporin 1 |
| LIX1 | limb and CNS expressed 1 |
| SCARA3 | scavenger receptor class A member 3 |
| KLF15 | krupel like factor 15 |
| SLC47A | solute carrier family 47 member 1 |
| WNK2 | WNK lysine deficient protein kinase 2 |
| PLVAP | plasmalemma vesicle associated protein |
| SHISA3 | shisa family member 2 |
| GALNT9 | polypeptide N-acetylgalactosaminyltransferase 9 |
| RGS16 | regulator of G protein signaling 16 |
| PARM1 | prostate androgen-regulated mucin-like protein |
| CASR | calcium sensing receptor |
| RPP25 | ribonuclease P and MRP subunit p25 |

Examination of the genes listed in Tables 14 and 15 showed that the top up-regulated DEG that specifically distinguished SRC from BRC0 included genes mediating aspects of cell cycle control (CDKN2A, IL11, TGFβ2), extracellular matrix reorganization (FN1I, CRISPLD2, COL1A1, LOX) and signaling pathways involved in development (RUNX2, LFNG, BDNF). The top down-regulated DEG that specifically distinguished SRC from BRC0 included genes involved in tight junction assembly and organization (CLDN3), glucose metabolism (UPP1, KLF 15), protein glycosylation (GYLTLl1B, MAN1C1, GALNT9) and water and ion transport (AQP1, SLC47A1, WNK2, CASR) as well as signaling pathways involved in development (RAI2, PLVAP, SHISA3, PARM1, CASR). Other noteworthy up-regulated DEG (not shown in Table 14 but still highly significant) included BDNF, FGF11, FOXE1, WNT5A, WNT10A, TGFβ1 and IGFBP3, all of which are involved in aspects of cell-cell signaling related to development and morphogenesis.

Of these genes, it is likely that the expression of CDKN2A is simply a manifestation of extended passaging of the cell population in vitro, as this gene is an established marker of cellular senescence (Famulski & Halloran, 2005; Molecular events in kidney ageing, Curr Opin Nephrol Hyperten 14:243-248).

With respect to IL11, although it is intimately involved in multiple aspects of fibrotic and inflammatory disease in kidney and other organs (Cook and Schafer, 2020; Hiding in Plain Sight: Interleukin-11 Emerges as a Master Regulator of Fibrosis, Tissue Integrity, and Stromal Inflammation, Ann Rev Med. 71: 263), it has a potential role in regenerative bioactivity that is of particular relevance. IL11 has been shown to be required for organ regeneration in Xenopus, through the induction and maintenance of undifferentiated progenitor populations across multiple cell lineages during tail regeneration (Tsujioka et al., 2017; interleukin-11 induces and maintains progenitors of different cell lineages during Xenopus tadpole tail regeneration. Nature Commun. 8: 495).

The TGFβ2 gene as well as other members of the TGFβ superfamily are known to be important mediators of kidney development, regulating nephron numbers either directly or indirectly (Sims-Lucas et al., 2008, Augmented and accelerated nephrogenesis in TGF-β2 heterozygous mutant mice, Pedr. Res 63: 607-612). Furthermore, TGF-β2, acting synergistically with FGF2, has been shown to induce multiple tubule formation in isolated rodent metanephric mesenchyme explants (Plisov et al., 2001. TGF beta 2, LIF and FGF2 cooperate to induce nephrogenesis, Development 128: 1045).

The branching morphogen CRISPLD2 (also known as LDL1) has been shown to have a specific localization pattern to the developing ureteric branch tip (UBT) (Rutledge et al., 2017. Cellular heterogeneity in the ureteric progenitor niche and distinct profiles of branching morphogenesis in organ development, Development 144:3177). Observations of expression of CRISPLD2/LDL1 suggested that CRISPLD2 secreted from metanephric mesenchymal cells enhances branching morphogenesis of the renal collecting systems, which parallels its putative effects on the early stages of lung bud arborization (Quinlan et al., 2007. LGL1, a novel branching morphogen in developing kidney, is induced by retinoic acid. Am J Physiol Renal Physiol; 293(4):F987-93).

The LOX and LOX-like family of proteins are known to play multiple roles in development and organogenesis through their bioactivity in the assembly and reorganization of the extracellular matrix. LOX and LOX-like proteins have been implicated in the development of multiple tissues and organs, including cerebrum, spinal cord, lung, heart, aorta, tooth, bone, cartilage, muscle and tendon, skin and uterus (Wei et al., 2020; Role of the lysyl oxidase family in organ development (Review), Exp. Ther. Med. 20: 163-172).

LFNG is one of three Notch ligands whose expression is involved in establishing and demarcating proximal/distal polarity in the renal vesicle. The Notch pathway establishes a proximal polarity that is carried through the comma- and S-shaped body stages and is integral for proximal tubule and podocyte development (0-Brian and McMahon, 2014. Induction and patterning of the metanephric nephron. Semin Cell Dev Biol. 36:31-38). Expression of LFNG and the closely related glycosyl transferase radicalfringe have been observed in the dorso-anterior region of the proximal pronephros at the tail bud stages of development in Xenopus. Expression of LFNG has been shown to be absent from the distal and proximal parts of the developing nephron in E14.5 mouse embryos, but strongly detected in the future proximal tubule (similar to expression of Notch ligand DLL1). And by E17.5, LFNG expression was shown to be restricted to portions of developing nephrons in the kidney periphery, suggesting that LFNG may regulated by NOTCH signaling in the cells of the future proximal tubule (Leimeister et al., 2003. Expression of Notch pathway genes in the embryonic mouse metanephros suggests a role in proximal tubule development. Gene Expression Patterns 3: 595-598).

Expression analysis of BDNF in human fetal kidneys has shown BDNF is localized to the apical region of epithelial cells within the developing primitive glomerular structures and mesenchymal derived tubules. While BDNF has been detected in the distal tubules with differentiation, its expression has not been observed in the uninduced mesenchyme, suggesting that BDNF is not involved in early induction events but rather, in the later stages of nephron organization (Huber et al., 1996. Neurotrophins and neurotrophin receptors in human fetal kidney. Developmental biology 179: 369-381). Interestingly, BDNF has also been shown to mediate repair of podocytes in vitro and in vivo through a micro-RNA dependent increase in cytoskeletal actin polymerization (Li et al., 2015. BDNF repairs podocyte damage by microRNA-mediated increase of actin polymerization. J Pathol 235: 731-744). Morpholino-mediated knockdown of BDNF in zebrafish larvae resulted in abnormal glomerular morphology, reduction in the number of podocytes and misexpression of the podocyte markers nephrin and podocin (Endlich et al., 2018. BDNF: mRNA expression in urine cells of patients with chronic kidney disease and its role in kidney function. J Cell Mol Med. 22: 5265-5277).

WNT5A has been shown to be essential in kidney development; congenital kidney and urinary tract abnormalities (CAKUT) in patients presenting with Robinow syndrome are associated with mutations in the WNT5A gene. Abnormalities in ureteric tree development, tubular epithelial cell organization and basement membrane integrity have also been observed in WNT5A deficient rodents (Pietila et al., 2016. Wnt5a Deficiency Leads to Anomalies in Ureteric Tree Development, Tubular Epithelial Cell Organization and Basement Membrane Integrity Pointing to a Role in Kidney Collecting Duct Patterning. Plos One 11: e0147171) as well as duplicated ureters and kidneys due to ectopic Ureteric Bud (UB) induction. During initial UB formation, WNT5A mutant embryos have shown dysregulated positioning of the Metanephric Mesenchyme (MM), resulting in spatiotemporally aberrant interaction between the MM and Wolffian duct, causing inappropriate GDNF signaling with the Wolfian duct. Furthermore, cell proliferation in the mutant MM has been shown to be significantly reduced relative to controls, suggesting a critical role of WNT5A/ROR2 signaling in morphogenesis of the MM to ensure correct epithelial tubule formation of the developing ureteric bud (Nishita et al., 2014. Role of Wnt5a-Ror2 Signaling in Morphogenesis of the Metanephric Mesenchyme during Ureteric Budding; Mol Cell Biol 16: 3096-3105). WNT5A signaling may also be involved in podocyte development and recovery of glomerular podocytes from injury by reorganization of the actin cytoskeleton through the planar cell polarity pathway (Babayeva et al., 2011. Planar cell polarity pathway regulates actin rearrangement, cell shape, motility, and nephrin distribution in podocytes. AJPRP F549-60).

IGFBP3 has been documented in the mature and differentiated cells of the collecting system and ureteric duct in human fetal kidney, likely acting to modulate the bioactivity of IGFs during nephrogenesis (Matsell et al., 1994. Expression of insulin-like growth factor and binding protein genes during nephrogenesis. Kidney Int 46: 1031-42).

Results/GO-analysis of DEG gene set shows enrichment of classifiers associated with development: A Protein ANalysis THrough Evolutionary Relationships (PANTHER) over-representation analysis was performed to identify GO categories specifically enriched in the DEG gene set relative to the human genome as a whole. Results of the PANTHER analysis were broadly consistent with the results shown in Tables 14 and 15, with the DEG dataset showing enrichment of GO-identifiers associated with development, including cell communication, cell differentiation, cell-cell adhesion, developmental process, nervous system development and system development. Key pathways highlighted in REACTOME pathway analysis of the overall DEG set included developmental biology, cell cycle, cell-cell communication, extracellular matrix organization, neuronal system, signal transduction, gene expression (transcription) pathways.

An analysis of GO-BP (biological process), GO-CC (cellular component) and GO-MF (molecular function) of all DEG in SRC relative to BRC0 in comparison to the whole human genome was also performed. Specific enrichment of DEG relative to whole human genome was observed for the following GO-BP categories associated with development: cellular process, single organism process, biological regulation, metabolic process, response to stimulus, multicellular organismal process, developmental process, signaling, localization, cellular component organization or biogenesis, multi-organism process, immune system process, reproduction, reproductive process, locomotion, biological adhesion, behavior, presynaptic process involved in chemical synaptic transmission, cell killing and cell aggregation. Consistent with prior reports showing that exosome mediated cell-cell communication is a significant mechanism of action for SRC (Bruce et al., 2013. Ex vivo culture and separation of functional renal cells. Methods Mol Biol 1001:53-64), enrichment of the following GO-CC categories in the SRC/BRC0 DEG set was noted: extracellular region, extracellular region part, supramolecular complex, synapse, other organism and other organism part. Finally, enrichment of DEG relative to whole human genome was observed for the following GO-MF categories: structural molecule activity, molecular function regulator, transporter activity, nucleic acid binding transcription factor activity, molecular transducer activity. Additional details regarding the gene compositions and ancestor/children of each of these GO classifiers may be found at amigo.geneontology.org.

Results/KEGG-analysis of DEG gene set shows enrichment of classifiers associated with development: KEGG annotation of the major identified metabolic and signal transduction pathways associated with the SRC/BRC0 DEG gene set was also performed. KEGG cellular process classifiers specifically enriched in the DEG gene set were broadly associated with epithelial cell junction formation, organization and maintenance, cell cycle control and regulation of stem cell pluripotency: Adherens junction, p53 signaling pathway, tight junction, signaling pathways regulating pluripotency of stem cells, regulation of action cytoskeleton, endocytosis, cell cycle and focal adhesion. In addition, multiple key signal transduction pathways associated with development, cell cycle control and regeneration were also enriched in key KEGG categories: AMPK, mTOR, WNT, HIF-1, JAK-STAT, RAP-1, TNF, TGFβ, MAPK, FOXO, HIPPO and PI3K-AKT signaling pathways, as well as cytokine-cytokine receptor interaction, ECM receptor interaction.

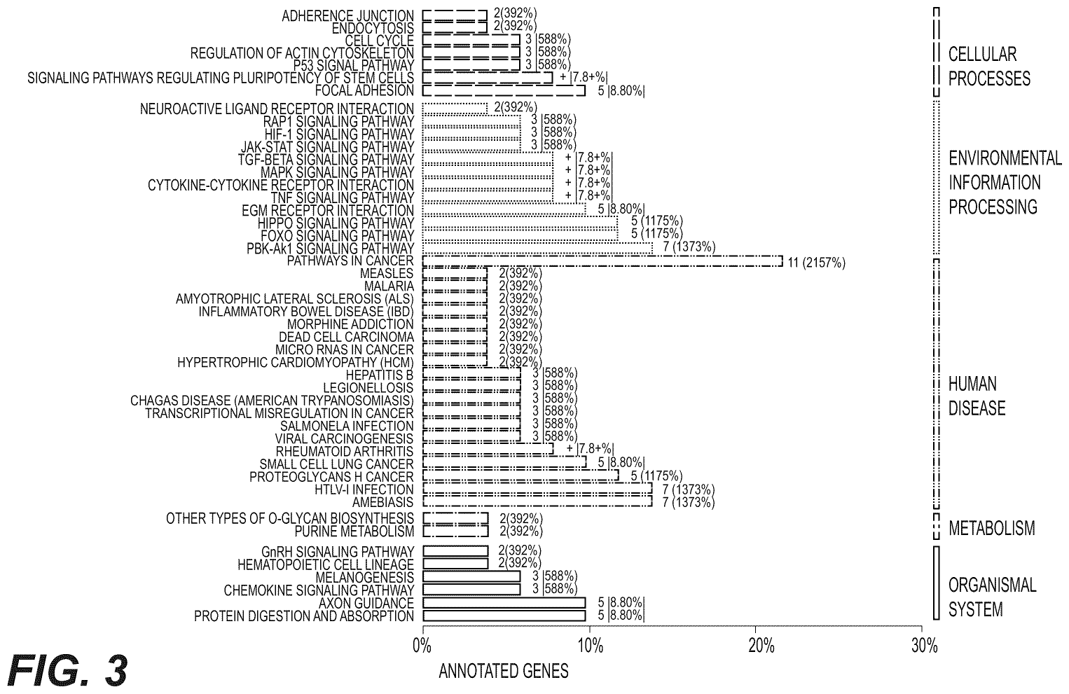

The scatter plot shown as FIG. 2 is a graphical representation of the KEGG enrichment analysis results. In this figure, the degree of KEGG enrichment was measured by rich factor, q-value, and the number of genes enriched in a particular pathway. The rich factor refers to the ratio of the number of differentially expressed genes located in the pathway to the total number of annotated genes located in the pathway. The larger the rich factor, the greater the degree of enrichment. Q-value is the corrected p-value after the multiple hypothesis test. The value range of q-value is [0,1], the closer to zero, the more significant the enrichment is. Key KEGG pathways of relevance to kidney regeneration included TGF-β signaling pathway, HIPPO signaling pathway, FOXO signaling pathway, Cell cycle, ECM-receptor interaction, pyrimidine metabolism.

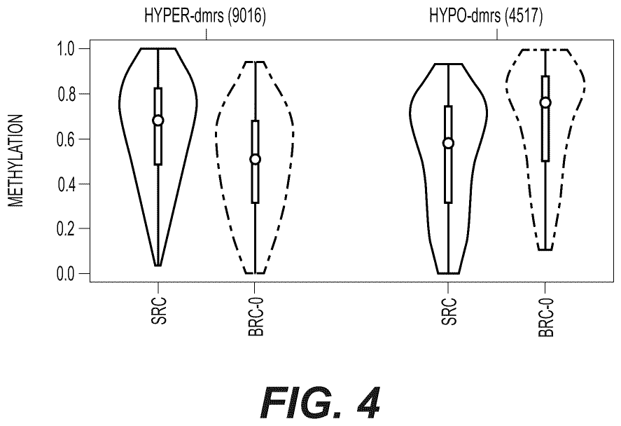

A specific focus on the up-regulated DEG set revealed the following KEGG classifiers to be significant: adherens junction, endocytosis, cell cycle, regulation of actin cytoskeleton, p53 signaling pathway, signaling pathways regulating pluripotency of stem cells and focal adhesion. Additional pathways of significance in the up-regulated DEG set included: Rap1 signaling pathway, HIF1 signaling pathway, JAK-STAT signaling pathway, TGF-β signaling pathway, MAPK signaling pathway, TNF signaling pathway and HIPPO signaling pathway (FIG. 3). A topGO analysis of the up-regulated DEG gene set is shown in FIG. 3. Key GO-BP identifiers especially pertinent to regeneration included: in utero embryonic development, epidermal growth factor receptor signaling pathway, G-protein coupled receptor signaling pathway, Notch signaling pathway and negative regulation of epithelial cell proliferation.

Specific examination of the down-regulated DEG set showed enrichment for the following KEGG classifiers: focal adhesion, apoptosis, lysosome, cell cycle, tight junction and endocytosis as well as signaling pathways: HIPPO, Hedgehog and AMPK. A topGO analysis of the downregulated DEG set showed enrichment for the following BP-classifiers: EGFR signaling pathway, Notch signaling pathway, GPCR signaling pathway, digestive tract morphogenesis, heart looping, negative regulation of epithelial cell proliferation.

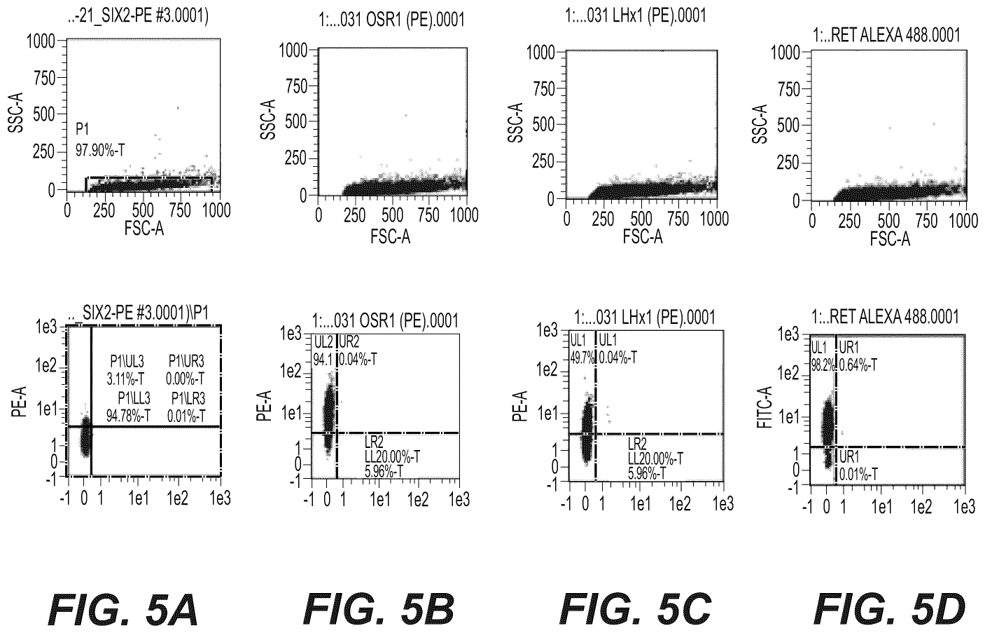

Results/Epigenomic profiling of SRC and BRC0 populations: The differentially methylated regions (DMR) comparison between SRC and BRC0 is presented as a violin boxplot in FIG. 4. It shows that the SRC genome had proportionally greater hypermethylation and less hypomethylation than to BRC0 genome (n=4). Based on the results of DMR annotation, functional enrichment analysis was performed on genes whose gene body and upstream and downstream regions (upstream-2 k, gene-body, and downstream-2 k) overlapped with DMR. The most highly enriched GO-BP classifiers in DMR-associated genes in SRC over BRC0 included cell adhesion, signal transduction and negative/positive regulation of transcription by RNA Polymerase. KEGG pathways that were identified as being highly enriched in SRC populations relative to BRC0 included: regulation of actin cytoskeleton, proteoglycans in cancer, focal adhesion, endocytosis, cAMP signaling pathway, RAS signaling pathway, RAP1 signaling pathway, PI3-AKT signaling pathway, MAPK signaling pathway and HIPPO signaling. Broadly, these classifiers were diagnostic of genes involved in extracellular matrix organization and reorganization, and signaling pathways associated with organismal development and cell proliferation. These classifiers were also consistent with classifiers identified previously in analysis of the DEG set.

Similarly, based on the distribution of DMR on the genome, there were overlapping genes for functional enrichment analysis with the gene promoter region (1.5K upstream from the transcription start site and 0.5K downstream of the transcription start site). GO-classifiers associated with DMR-related genes according to this analysis included regulation of actin cytoskeleton, endocytosis, cAMP signaling pathway, cell adhesion molecules CAM, RAP1 signaling pathway. Also noteable for the greatest fold change in SRC relative to BRC0 was leukocyte trans-endothelial migration, a critical inflammatory stage associated with wound healing and tissue regeneration. Furthermore, KEGG-classifiers associated with DMR-related genes included: regulation of actin cytoskeleton, cAMP signaling pathway, signaling pathways regulating pluripotency of stem cells, RAS signaling pathway, RAP1 signaling pathway, PI3K-AKT signaling pathway, endocytosis, cell adhesion CAM, leukocyte trans-endothelial pathways. These classifiers were also broadly consistent with those identified previously in the DEG set.

Results/Summary: Overall, human sourced SRC presented a DEG set enriched in GO and KEGG classifiers that were associated with development. KEGG cellular process classifiers specifically enriched in the DEG gene set were broadly associated with epithelial cell junction formation, organization and maintenance, cell cycle control and regulation of stem cell pluripotency. A similar pattern of enrichment was observed in the DMR gene set. On the basis of these and other expression data presented herein, SRCs may mediate in vivo regeneration of kidney and improve clinical outcomes of kidney disease patients by, in part, activating mechanistic pathways that parallel aspects of embryonic nephrogenesis that ultimately trigger neo-kidney-like tissue formation at sites of injury or disease.

### Example 5: Enriched Heterogeneous Renal Cell Populations Administered to Moderate to Advanced Type 2 Diabetes-Related CKD Subjects—Assessment of Nephrogenic Marker Expression Levels and Potential Relationship to Clinical Outcomes

Introduction: With the understanding that enriched heterogeneous renal cell populations, e.g., SRCs as referred to in this Example, may be mediating regenerative effects on diseased kidneys by influencing signaling cascades that mediate neo-nephrogenesis, SRCs administered to clinical trial subjects with moderate to advanced type-2 diabetes-related CKD were assessed for expression levels of nephrogenic markers, and were correspondingly compared to patients' clinical responses.

Materials and Methods/Preparation of SRCs for Administration: All patients in the clinical trial underwent a standard percutaneous kidney biopsy to isolate kidney cells from which to prepare their SRCs for administration. SRCs were prepared from the kidney biopsies as described in Examples 1 and 4.

Materials and Methods/Phenotypic Marker Analysis of SRCs: SRCs in suspension were centrifuged at 300×g for 5 min at room temperature. The resulting SRC pellet was washed once with Dulbecco's phosphate-buffered saline (DPBS) and then fixed in BD Fixation/Permeabilization Solution (BD Biosciences, Cat. No. 554714) for 20 min at 4° C. Fixed cells were centrifuged at 500×g for 5 min at room temperature. The cell pellet was resuspended in BD Perm/Wash buffer. Resuspended cells were aliquoted (20 μL containing 150,000 cells) before incubation with 1 μg of primary antibody for 1 hr at 4° C. At the end of this incubation, cells were pelleted, washed with BD Perm/Wash buffer, pelleted again, and resuspended in 150 μL of DPBS. For antibodies not directly conjugated to a fluorophore, 1 μg of secondary antibody was incubated with the cells for an additional 30 minutes before washing and resuspending in DPBS. Cells were then analysed by flow cytometry.

Materials and Methods/FACS Analysis of SRCs: To determine if the SRCs contained markers for cap mesenchyme, nephrogenic interstitium, and ureteric bud, the cells were screened for membrane proteins that identified these specific constituents. The following immunoreagents were used for the screening: Goat anti-rabbit IgG H&L, Alexafluor 488 (Abcam Inc. Cat #ab150077); Rabbit polyclonal to NPHS2 (Abcam Inc. Cat #ab50339); Rabbit monoclonal (Y17R) to Nephrin (Abcam Inc. Cat #ab136894); Alexafluor 488 rabbit monoclonal (EPR2871) to RET (Abcam Inc. ab237105); Rabbit IgG, polyclonal, isotype control (Abcam Inc. ab37415); Six2 Antibody (H-4) PE (Santa Cruz, Cat #sc-377193 PE); OSR1 Antibody (Santa Cruz, Cat #sc376545 PE); Lhxl monoclonal IgG2a (Santa Cruz, Cat #sc515631 PE); RET rabbit monoclonal antibody (Abcam Inc. Cat #ab237105); Goat polyclonal secondary antibody to RB IGG (H&L)-Alexa Fluor 488 (Abcam Inc. Cat #ab150077); Alexa Fluor® 647 Mouse IgG2a, κ Isotype Control (BD, Cat #557715); FgfS monoclonal, full length (Abcam, Cat #ab89550); Rabbit IgG Isotype Control [PE] (Novus Biologicals, Cat #NBP2-36463PE), and PE Mouse IgG1, κ Isotype Control (BD, Cat #555749).

Materials and Methods/FACS phenotypic marker analysis: Phenotypic marker analysis was performed by pelleting at least 50,000 SRCs in a 1.5 mL microcentrifuge tube and fixing the cells in 100 uL of BD Fixation/Permeabilization Solution for 20 min at 4° C. The microcentrifuge tubes were then placed in a microcentrifuge at 500×g for 3 minutes to pellet the fixed cells, which were then washed once with 500 uL of BD Perm/Wash buffer. The pellet of the wash was resuspended in 20 μL of BD Perm/Wash buffer and 1 ug primary antibody was added for incubation for 1 hr at 4° C. The cells were washed once with 500 μL of BD Perm/Wash buffer and resuspended in 20 μL of BD Perm/Wash buffer with addition of 1 μg secondary antibody for incubation for 30 min at 4° C. A last wash was performed with 500 uL of BD Perm/Wash buffer. The pellet was resuspended in 150 uL DPBS and the cells were analysed by flow cytometry.

Results/Marker expression analysis of SRCs: Results of marker expression analysis of SRCs administered to subjects in the clinical trials is provided in Table 16.

TABLE 16
Complied data for FACS analysis

| Marker/ | | | | | | | | |
|---|---|---|---|---|---|---|---|---|
| percent | | | | | | | | |
| positive | Six2 | OSR1 | RET | LHx1 | Nephrin | Podocine | FGF8 | Rack1 |
| SRCs | 10.3 ± 3.87 | 83.2 ± 6.6 | 42.6 ± 9.35 | 71.1 ± 12.8 | 79.5 | No obs | 2.79 ± 1.12 | 92.6 ± 2.83 |
| moderate/high | (n = 8) | (n = 8) | (n = 8) | (n = 8) | (n = 1) | | (n = 8) | (n = 6) |
| responders | | | | | | | | |
| SRCs low | 2.31 ± 1.00 | 69.7 ± 4.04 | 24.6 ± 8.46 | 23.5 ± 7.77 | 86.5 ± 3.6 | 96.5 ± 0.64 | 9.33 ± 5.93 | 95.9 ± 3.09 |
| responders | (n = 12) | (n = 12) | (n = 12) | (n = 12) | (n = 8) | (n = 4) | (n = 11) | (n = 3) |
| p-value | 0.08 | 0.11 | 0.17 | 0.008* | N/A | N/A | 0.30 | 0.46 |

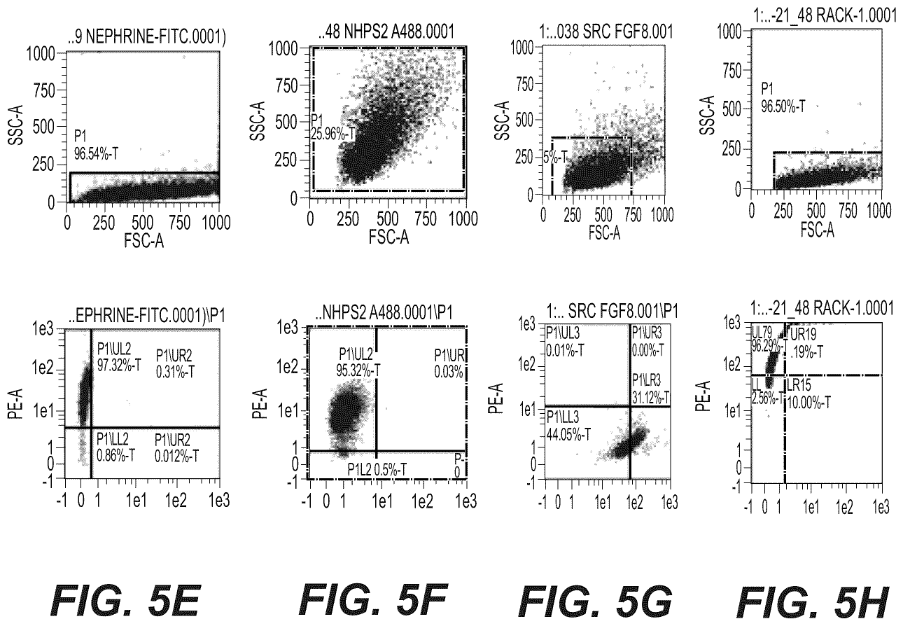

Consistent with the earlier analyses, e.g., presented in Example 2, the clinical trial subjects' SRCs expressed markers of the nephrogenic marker panel, and other markers including nephrin, podocin and RACK-1. The clinical trial subjects' SRCs also included similar percentages of cells expressing the markers as was earlier described. Correlation of various markers found evidence for their co-expression (see FIG. 5A-H).

Further analysis of the expression of the markers by the SRCs revealed that subjects identified as moderate/high responders (improvement in eGFR) relative to those who were identified as low responders (improved eGFR slope, but without improvement in eGFR) showed greater expression of various biomarkers trending towards significance for Six2, and with significance for LHx1 (Table 16). These analyses provide further evidence that SRCs may be mediating neo-nephrogenesis by demonstrating that as cells of SRC populations have higher levels of expression of nephrogenic markers, they may have increased capacity to mediate regenerative and restorative activities, thereby providing therapeutic effects to diseased kidneys.

1. A method of identifying an enriched heterogeneous renal cell population as having a therapeutic potential, comprising:
determining whether cells of the enriched heterogeneous renal cell population express at least one nephrogenic marker; and
identifying the enriched heterogeneous renal cell population as having therapeutic potential if cells of the enriched heterogeneous renal cell population are determined to express the at least one nephrogenic marker;
wherein the at least one nephrogenic marker comprises one or more of SIX2, OSR1, LHX1, RET and FGF8.

2. The method of claim 1, wherein the step of determining comprises determining percentage of cells of the enriched heterogeneous renal cell population that express the at least one nephrogenic marker.

3. The method of claim 1, wherein the at least one nephrogenic marker comprises SIX2.

4. The method of claim 2, wherein the at least one nephrogenic marker comprises SIX2 and
wherein the enriched heterogeneous renal cell population is identified as having a therapeutic potential if greater than 0% but no more than about 6.0% of cells of the enriched heterogeneous renal cell population express SIX2.

5. The method of claim 1, wherein the at least one nephrogenic marker comprises OSR1.

6. The method of claim 2, wherein the at least one nephrogenic marker comprises OSR1 and
wherein the enriched heterogeneous renal cell population is identified as having a therapeutic potential if about 36% to about 85% of cells of the enriched heterogeneous renal cell population express OSR1.

7. The method of claim 1, wherein the at least one nephrogenic marker comprises LHX1.

8. The method of claim 2, wherein the at least one nephrogenic marker comprises LHX1 and
wherein the enriched heterogeneous renal cell population is identified as having a therapeutic potential if about 8% to about 58% of cells of the enriched heterogeneous renal cell population are determined to express LHX1.

9. The method of claim 1, wherein the at least one nephrogenic marker comprises RET.

10. The method of claim 2, wherein the at least one nephrogenic marker comprises RET and
wherein the enriched heterogeneous renal cell population is identified as having a therapeutic potential if about 49% to about 90% of cells of the enriched heterogeneous renal cell population express RET.

11. The method of claim 1, wherein the at least one nephrogenic marker comprises FGF8.

12. The method of claim 2, wherein the at least one nephrogenic marker comprises FGF8 and
wherein the enriched heterogeneous renal cell population is identified as having a therapeutic potential if about 0.48% to about 59% of cells of the enriched heterogeneous renal cell population express FGF8.

13. The method of claim 1, wherein the at least one nephrogenic marker comprises:
(a) SIX2 and OSR1; or
(b) SIX2 and LHX1; or
(c) SIX2 and RET; or
(d) SIX2 and FGF8; or
(e) OSR1 and LHX1; or
(f) OSR1 and RET; or
(g) OSR1 and FGF8; or
(h) LHX1 and RET; or
(i) LHX1 and FGF8; or
(j) RET and FGF8.

14. The method of claim 2, wherein the at least one nephrogenic marker comprises:
(a) SIX2 and OSR1, and
wherein the enriched heterogeneous renal cell population is identified as having a therapeutic potential if it is determined that greater than 0% and at most about 6% of cells of the enriched heterogeneous renal cell population express SIX2 and at least about 36% of cells of the enriched heterogeneous renal cell population express OSR1; or
(b) SIX2 and LHX1, and
wherein the enriched heterogeneous renal cell population is identified as having a therapeutic potential if it is determined that greater than 0% and at most about 6% of cells of the enriched heterogeneous renal cell population express SIX2 and greater than about 8% of cells of the enriched heterogeneous renal cell population express LHX1; or
(c) SIX2 and RET, and
wherein the enriched heterogeneous renal cell population is identified as having a therapeutic potential if it is determined that greater than 0% and at most about 6% of cells of the enriched heterogeneous renal cell population express SIX2 and greater than about 49% of cells of the enriched heterogeneous renal cell population express RET; or
(d) SIX2 and FGF8, and
wherein the enriched heterogeneous renal cell population is identified as having a therapeutic potential if it is determined that greater than 0% and at most about 6% of cells of the enriched heterogeneous renal cell population express SIX2 and greater than 0% and at most about 59% of cells of the enriched heterogeneous renal cell population express FGF8; or
(e) OSR1 and LHX1, and
wherein the enriched heterogeneous renal cell population is identified as having a therapeutic potential if it is determined that at least about 36% of cells of the enriched heterogeneous renal cell population express OSR1 and greater than about 8% of cells of the enriched heterogeneous renal cell population express LHX1; or
(f) OSR1 and RET, and
wherein the enriched heterogeneous renal cell population is identified as having a therapeutic potential if it is determined that at least about 36% of cells of the enriched heterogeneous renal cell population express OSR1 and greater than about 49% of cells of the enriched heterogeneous renal cell population express RET; or
(g) OSR1 and FGF8, and
wherein the enriched heterogeneous renal cell population is identified as having a therapeutic potential if it is determined that at least about 36% of cells of the enriched heterogeneous renal cell population express OSR1 and greater than 0% and at most about 59% of cells of the enriched heterogeneous renal cell population express FGF8; or
(h) LHX1 and RET, and
wherein the enriched heterogeneous renal cell population is identified as having a therapeutic potential if it is determined that greater than about 8% of cells of the enriched heterogeneous renal cell population express LHX1 and greater than about 49% of cells of the enriched heterogeneous renal cell population express RET; or
(i) LHX1 and FGF8, and
wherein the enriched heterogeneous renal cell population is identified as having a therapeutic potential if it is determined that greater than about 8% of cells of the enriched heterogeneous renal cell population express LHX1 and greater than 0% and at most about 59% of cells of the enriched heterogeneous renal cell population express FGF8; or
(j) RET and FGF8, and
wherein the enriched heterogeneous renal cell population is identified as having a therapeutic potential if it is determined that greater than about 49% of cells of the enriched heterogeneous renal cell population express RET and greater than 0% and at most about 59% of cells of the enriched heterogeneous renal cell population express FGF8.

15. The method of claim 2, wherein the at least one nephrogenic marker comprises:
(a) SIX2 and OSR1, and
wherein the enriched heterogeneous renal cell population is identified as having a therapeutic potential if it is determined that greater than 0% and at most about 6% of cells of the enriched heterogeneous renal cell population express SIX2 and about 36% to about 85% of cells of the enriched heterogeneous renal cell population express OSR1; or
(b) SIX2 and LHX1, and
wherein the enriched heterogeneous renal cell population is identified as having a therapeutic potential if it is determined that greater than 0% and at most about 6% of cells of the enriched heterogeneous renal cell population express SIX2 and about 8% to about 58% of cells of the enriched heterogeneous renal cell population express LHX1; or
(c) SIX2 and RET, and
wherein the enriched heterogeneous renal cell population is identified as having a therapeutic potential if it is determined that greater than 0% and at most about 6% of cells of the enriched heterogeneous renal cell population express SIX2 and about 49% to about 90% of cells of the enriched heterogeneous renal cell population express RET; or
(d) SIX2 and FGF8, and
wherein the enriched heterogeneous renal cell population is identified as having a therapeutic potential if it is determined that greater than 0% and at most about 6% of cells of the enriched heterogeneous renal cell population express SIX2 and about 0.48% to about 59% of cells of the enriched heterogeneous renal cell population express FGF8; or
(e) OSR1 and LHX1, and
wherein the enriched heterogeneous renal cell population is identified as having a therapeutic potential if it is determined that about 36% to about 85% of cells of the enriched heterogeneous renal cell population express OSR1 and about 8% to about 58% of cells of the enriched heterogeneous renal cell population are determined to express LHX1; or
(f) OSR1 and RET, and
wherein the enriched heterogeneous renal cell population is identified as having a therapeutic potential if it is determined that about 36% to about 85% of cells of the enriched heterogeneous renal cell population express OSR1 and about 49% to about 90% of cells of the enriched heterogeneous renal cell population express RET; or
(g) OSR1 and FGF8, and
wherein the enriched heterogeneous renal cell population is identified as having a therapeutic potential if it is determined that about 36% to about 85% of cells of the enriched heterogeneous renal cell population express OSR1 and about 0.48% to about 59% of cells of the enriched heterogeneous renal cell population express FGF8; or
(h) LHX1 and RET, and
wherein the enriched heterogeneous renal cell population is identified as having a therapeutic potential if it is determined that about 8% to about 58% of cells of the enriched heterogeneous renal cell population are determined to express LHX1 and about 49% to about 90% of cells of the enriched heterogeneous renal cell population express RET; or
(i) LHX1 and FGF8, and
wherein the enriched heterogeneous renal cell population is identified as having a therapeutic potential if it is determined that about 8% to about 58% of cells of the enriched heterogeneous renal cell population express LHX1 and about 0.48% to about 59% of cells of the enriched heterogeneous renal cell population express FGF8; or
(j) RET and FGF8, and
wherein the enriched heterogeneous renal cell population is identified as having a therapeutic potential if it is determined that about 49% to about 90% of cells of the enriched heterogeneous renal cell population express RET and about 0.48% to about 59% of cells of the enriched heterogeneous renal cell population express FGF8.

16. The method of claim 1, wherein the at least one nephrogenic marker comprises:
(a) SIX2, OSR1 and LHX1; or
(b) SIX2, OSR1 and RET; or
(c) SIX2, OSR1 and FGF8; or
(d) SIX2, LHX1 and RET; or
(e) SIX2, LHX1 and FGF8; or
(f) SIX2, RET and FGF8; or
(g) OSR1, LHX1 and RET; or
(h) OSR1, LHX1 and FGF8; or
(i) OSR1, RET and FGF8; or
(j) LHX1, RET and FGF8.

17. The method of claim 2, wherein the at least on nephrogenic marker comprises:
(a) SIX2, OSR1 and LHX1, and
wherein the enriched heterogeneous renal cell population is identified as having a therapeutic potential if it is determined that greater than 0% and at most about 6% of cells of the enriched heterogeneous renal cell population express SIX2, at least about 36% of cells of the enriched heterogeneous renal cell population express OSR1 and greater than about 8% of cells of the enriched heterogeneous renal cell population express LHX1; or
(b) SIX2, OSR1 and RET, and
wherein the enriched heterogeneous renal cell population is identified as having a therapeutic potential if it is determined that greater than 0% and at most about 6% of cells of the enriched heterogeneous renal cell population express SIX2, at least about 36% of cells of the enriched heterogeneous renal cell population express OSR1 and greater than about 49% of cells of the enriched heterogeneous renal cell population express RET; or
(c) SIX2, OSR1 and FGF8, and
wherein the enriched heterogeneous renal cell population is identified as having a therapeutic potential if it is determined that greater than 0% and at most aboutg 6% of cells of the enriched heterogeneous renal cell population express SIX2, at least about 36% of cells of the enriched heterogeneous renal cell population express OSR1 and greater than 0% and at most about 59% of cells of the enriched heterogeneous renal cell population express FGF8; or
(d) SIX2, LHX1 and RET, and
wherein the enriched heterogeneous renal cell population is identified as having a therapeutic potential if it is determined that greater than 0% and at most about 6% of cells of the enriched heterogeneous renal cell population express SIX2, greater than about 8% of cells of the enriched heterogeneous renal cell population express LHX1 and greater than about 49% of cells of the enriched heterogeneous renal cell population express RET; or
(e) SIX2, LHX1 and FGF8, and
wherein the enriched heterogeneous renal cell population is identified as having a therapeutic potential if it is determined that greater than 0% and at most about 6.0% of cells of the enriched heterogeneous renal cell population express SIX2, greater than about 8% of cells of the enriched heterogeneous renal cell population express LHX1 and greater than 0% and at most about 59% of cells of the enriched heterogeneous renal cell population express FGF8; or
(f) SIX2, RET and FGF8, and
wherein the enriched heterogeneous renal cell population is identified as having a therapeutic potential if it is determined that greater than 0% and at most about 6% of cells of the enriched heterogeneous renal cell population express SIX2, greater than about 49% of cells of the enriched heterogeneous renal cell population express RET and greater than 0% and at most about 59% of cells of the enriched heterogeneous renal cell population express FGF8; or
(g) OSR1, LHX1 and RET, and
wherein the enriched heterogeneous renal cell population is identified as having a therapeutic potential if it is determined that at least about 36% of cells of the enriched heterogeneous renal cell population express OSR1, greater than about 8% of cells of the enriched heterogeneous renal cell population express LHX1 and greater than about 49% of cells of the enriched heterogeneous renal cell population express RET; or
(h) OSR1, LHX1 and FGF8, and
wherein the enriched heterogeneous renal cell population is identified as having a therapeutic potential if it is determined that at least about 36% of cells of the enriched heterogeneous renal cell population express OSR1, greater than about 8% of cells of the enriched heterogeneous renal cell population express LHX1 and greater than 0% and at most about 59% of cells of the enriched heterogeneous renal cell population express FGF8; or
(i) OSR1, RET and FGF8, and
wherein the enriched heterogeneous renal cell population is identified as having a therapeutic potential if it is determined that at least about 36% of cells of the enriched heterogeneous renal cell population express OSR1, greater than about 49% of cells of the enriched heterogeneous renal cell population express RET and greater than 0% and at most about 59% of cells of the enriched heterogeneous renal cell population express FGF8; or
(j) LHX1, RET and FGF8, and
wherein the enriched heterogeneous renal cell population is identified as having a therapeutic potential if it is determined that greater than about 8% of cells of the enriched heterogeneous renal cell population express LHX1, greater than about 49% of cells of the enriched heterogeneous renal cell population express RET and greater than 0% and at most about 59% of cells of the enriched heterogeneous renal cell population express FGF8.

18. The method of claim 2, wherein the at least one nephrogenic marker comprises:
(a) SIX2, OSR1 and LHX1, and
wherein the enriched heterogeneous renal cell population is identified as having a therapeutic potential if it is determined that greater than 0% and at most about 6% of cells of the enriched heterogeneous renal cell population express SIX2, about 36% to about 85% of cells of the enriched heterogeneous renal cell population express OSR1 and about 8% to about 58% of cells of the enriched heterogeneous renal cell population are determined to express LHX1; or
(b) SIX2, OSR1 and RET, and
wherein the enriched heterogeneous renal cell population is identified as having a therapeutic potential if it is determined that greater than 0% and at most about 6% of cells of the enriched heterogeneous renal cell population express SIX2, about 36% to about 85% of cells of the enriched heterogeneous renal cell population express OSR1 and about 49% to about 90% of cells of the enriched heterogeneous renal cell population express RET; or
(c) SIX2, OSR1 and FGF8, and
wherein the enriched heterogeneous renal cell population is identified as having a therapeutic potential if it is determined that greater than 0% and at most about 6% of cells of the enriched heterogeneous renal cell population express SIX2, about 36% to about 85% of cells of the enriched heterogeneous renal cell population express OSR1 and about 0.48% to about 59% of cells of the enriched heterogeneous renal cell population express FGF8; or
(d) SIX2, LHX1 and RET, and
wherein the enriched heterogeneous renal cell population is identified as having a therapeutic potential if it is determined that greater than 0% and at most about 6% of cells of the enriched heterogeneous renal cell population express SIX2, about 8% to about 58% of cells of the enriched heterogeneous renal cell population are determined to express LHX1 and about 49% to about 90% of cells of the enriched heterogeneous renal cell population express RET; or
(e) SIX2, LHX1 and FGF8, and
wherein the enriched heterogeneous renal cell population is identified as having a therapeutic potential if it is determined that greater than 0% and at most about 6% of cells of the enriched heterogeneous renal cell population express SIX2, about 8% to about 58% of cells of the enriched heterogeneous renal cell population are determined to express LHX1 and about 0.48% to about 59% of cells of the enriched heterogeneous renal cell population express FGF8; or
(f) SIX2, RET and FGF8, and
wherein the enriched heterogeneous renal cell population is identified as having a therapeutic potential if it is determined that greater than 0% at most about 6% of cells of the enriched heterogeneous renal cell population express SIX2, about 49% to about 90% of cells of the enriched heterogeneous renal cell population express RET and about 0.48% to about 59% of cells of the enriched heterogeneous renal cell population express FGF8; or
(g) OSR1, LHX1 and RET, and
wherein the enriched heterogeneous renal cell population is identified as having a therapeutic potential if it is determined that about 36% to about 85% of cells of the enriched heterogeneous renal cell population express OSR1, about 8% to about 58% of cells of the enriched heterogeneous renal cell population are determined to express LHX1 and about 49% to about 90% of cells of the enriched heterogeneous renal cell population express RET; or
(h) OSR1, LHX1 and FGF8, and
wherein the enriched heterogeneous renal cell population is identified as having a therapeutic potential if it is determined that about 36% to about 85% of cells of the enriched heterogeneous renal cell population express OSR1, about 8% to about 58% of cells of the enriched heterogeneous renal cell population are determined to express LHX1 and about 0.48% to about 59% of cells of the enriched heterogeneous renal cell population express FGF8; or
(i) OSR1, RET and FGF8, and
wherein the enriched heterogeneous renal cell population is identified as having a therapeutic potential if it is determined that about 36% to about 85% of cells of the enriched heterogeneous renal cell population express OSR1, about 49% to about 90% of cells of the enriched heterogeneous renal cell population express RET and about 0.48% to about 59% of cells of the enriched heterogeneous renal cell population express FGF8; or
(j) LHX1, RET and FGF8, and
wherein the enriched heterogeneous renal cell population is identified as having a therapeutic potential if it is determined that about 8% to about 58% of cells of the enriched heterogeneous renal cell population are determined to express LHX1, about 49% to about 90% of cells of the enriched heterogeneous renal cell population express RET and about 0.48% to about 59% of cells of the enriched heterogeneous renal cell population express FGF8.

19. The method of claim 1, wherein the at least one nephrogenic marker comprises:
(a) SIX2, OSR1, LHX1 and RET;
(b) SIX2, OSR1, LHX1 and FGF8;
(c) SIX2, LHX1, RET and FGF8;
(d) SIX2, OSR1, RET and FGF8;
(e) OSR1, LHX1, RET and FGF8.

20. The method of claim 2, wherein the at least one nephrogenic marker comprises:
(a) SIX2, OSR1, LHX1 and RET, and
wherein the enriched heterogeneous renal cell population is identified as having a therapeutic potential if at it is determined that greater than 0% and at most about 6% of cells of the enriched heterogeneous renal cell population express SIX2, at least about 36% of cells of the enriched heterogeneous renal cell population express OSR1, greater than about 8% of cells of the enriched heterogeneous renal cell population express LHX1 and greater than about 49% of cells of the enriched heterogeneous renal cell population express RET;
(b) SIX2, OSR1, LHX1 and FGF8, and
wherein the enriched heterogeneous renal cell population is identified as having a therapeutic potential if at it is determined that greater than 0% and at most about 6% of cells of the enriched heterogeneous renal cell population express SIX2, at least about 36% of cells of the enriched heterogeneous renal cell population express OSR1, greater than about 8% of cells of the enriched heterogeneous renal cell population express LHX1 and greater than 0% and at most about 59% of cells of the enriched heterogeneous renal cell population express FGF8;
(c) SIX2, LHX1, RET and FGF8, and
wherein the enriched heterogeneous renal cell population is identified as having a therapeutic potential if it is determined that greater than 0% and at most about 6% of cells of the enriched heterogeneous renal cell population express SIX2, greater than about 8% of cells of the enriched heterogeneous renal cell population express LHX1, greater than about 49% of cells of the enriched heterogeneous renal cell population express RET and greater than 0% and at most about 59% of cells of the enriched heterogeneous renal cell population express FGF8;
(d) SIX2, OSR1, RET and FGF8, and
wherein the enriched heterogeneous renal cell population is identified as having a therapeutic potential if at it is determined that greater than 0% and at most about 6% of cells of the enriched heterogeneous renal cell population express SIX2, at least about 36% of cells of the enriched heterogeneous renal cell population express OSR1, greater than about 49% of cells of the enriched heterogeneous renal cell population express RET and greater than 0% and at most about 59% of cells of the enriched heterogeneous renal cell population express FGF8;
(e) OSR1, LHX1, RET and FGF8, and
wherein the enriched heterogeneous renal cell population is identified as having a therapeutic potential if it is determined that at least about 36% of cells of the enriched heterogeneous renal cell population express OSR1, greater than about 8% of cells of the enriched heterogeneous renal cell population express LHX1, greater than about 49% of cells of the enriched heterogeneous renal cell population express RET and greater than 0% and at most about 59% of cells of the enriched heterogeneous renal cell population express FGF8.

21. The method of claim 2, wherein the at least one nephrogenic marker comprises:
(a) SIX2, OSR1, LHX1 and RET, and
wherein the enriched heterogeneous renal cell population is identified as having a therapeutic potential if it is determined that greater than 0% and at most about 6% of cells of the enriched heterogeneous renal cell population express SIX2, about 36% to about 85% of cells of the enriched heterogeneous renal cell population express OSR1, about 8% to about 58% of cells of the enriched heterogeneous renal cell population are determined to express LHX1 and about 49% to about 90% of cells of the enriched heterogeneous renal cell population express RET;
(b) SIX2, OSR1, LHX1 and FGF8, and
wherein the enriched heterogeneous renal cell population is identified as having a therapeutic potential if it is determined that greater than 0% and at most about 6% of cells of the enriched heterogeneous renal cell population express SIX2, about 36% to about 85% of cells of the enriched heterogeneous renal cell population express OSR1, about 8% to about 58% of cells of the enriched heterogeneous renal cell population are determined to express LHX1 and about 0.48% to about 59% of cells of the enriched heterogeneous renal cell population express FGF8;
(c) SIX2, LHX1, RET and FGF8, and
wherein the enriched heterogeneous renal cell population is identified as having a therapeutic potential if it is determined that greater than 0% and at most about 6% of cells of the enriched heterogeneous renal cell population express SIX2, about 8% to about 58% of cells of the enriched heterogeneous renal cell population are determined to express LHX1, about 49% to about 90% of cells of the enriched heterogeneous renal cell population express RET and about 0.48% to about 59% of cells of the enriched heterogeneous renal cell population express FGF8;
(d) SIX2, OSR1, RET and FGF8, and
wherein the enriched heterogeneous renal cell population is identified as having a therapeutic potential if it is determined that greater than 0% and at most about 6% of cells of the enriched heterogeneous renal cell population express SIX2, about 36% to about 85% of cells of the enriched heterogeneous renal cell population express OSR1, about 49% to about 90% of cells of the enriched heterogeneous renal cell population express RET and about 0.48% to about 59% of cells of the enriched heterogeneous renal cell population express FGF8;
(e) OSR1.LHX1, RET and FGF8, and
wherein the enriched heterogeneous renal cell population is identified as having a therapeutic potential if it is determined that about 36% to about 85% of cells of the enriched heterogeneous renal cell population express OSR1, about 8% to about 58% of cells of the enriched heterogeneous renal cell population are determined to express LHX1, about 49% to about 90% of cells of the enriched heterogeneous renal cell population express RET and about 0.48% to about 59% of cells of the enriched heterogeneous renal cell population express FGF8.

22. The method of claim 1, wherein the at least one nephrogenic marker comprises SIX2, OSR1, LHX1, RET and FGF8.

23. The method of claim 2, wherein the at least one nephrogenic marker comprises SIX2, OSR1, LHX1, RET and FGF, and
wherein the enriched heterogeneous renal cell population is identified as having a therapeutic potential if it is determined that greater than 0% and at most about 6% of cells of the enriched heterogeneous renal cell population express SIX2, at least about 36% of cells of the enriched heterogeneous renal cell population express OSR1, greater than about 8% of cells of the enriched heterogeneous renal cell population express LHX1, greater than about 49% of cells of the enriched heterogeneous renal cell population express RET and greater than 0% and at most about 59% of cells of the enriched heterogeneous renal cell population express FGF8.

24. The method of claim 2, wherein the at least one nephrogenic marker comprises SIX2, OSR1, LHX1, RET and FGF8, and
wherein the enriched heterogeneous renal cell population is identified as having a therapeutic potential if it is determined that greater than 0% and at most about 6% of cells of the enriched heterogeneous renal cell population express SIX2, about 36% to about 85% of cells of the enriched heterogeneous renal cell population express OSR1, about 8% to about 58% of cells of the enriched heterogeneous renal cell population are determined to express LHX1, about 49% to about 90% of cells of the enriched heterogeneous renal cell population express RET and about 0.48% to about 59% of cells of the enriched heterogeneous renal cell population express FGF8.

25. The method of any of claims 1-24, further comprising:
determining whether cells of the enriched heterogeneous renal cell population express one or more of nephrin, podocin or RACK-1; and
identifying the enriched heterogeneous renal cell population as having therapeutic potential if cells of the enriched heterogeneous renal cell population are determined to express one or more of nephrin, podocin or RACK-1.

26. The method of claim 25, wherein the one or more comprises nephrin.

27. The method of claim 25, wherein the one or more comprises podocin.

28. The method of claim 26, wherein the one or more further comprises podocin.

29. The method of claim 25, wherein the one or more comprises RACK-1.

30. The method of claim 26, wherein the one or more further comprises RACK-1.

31. The method of claim 27, wherein the one or more further comprises RACK-1.

32. The method of claim 28, wherein the one or more further comprises RACK-1.

33. The method of claim 26, wherein the enriched heterogeneous renal cell population is identified as having a therapeutic potential if it is determined that about 4% to about 99% of cells of the heterogeneous renal cell population express nephrin.

34. The method of claim 27, wherein the enriched heterogeneous renal cell population is identified as having a therapeutic potential if it is determined that at least about 90% of cells of the heterogeneous renal cell population express podocin.

35. The method of claim 28, wherein the enriched heterogeneous renal cell population is identified as having a therapeutic potential if it is determined that about 4% to about 99% of cells of the heterogeneous renal cell population express nephrin and at least about 90% of cells of the heterogeneous renal cell population express podocin.

36. The method of claim 29, wherein the enriched heterogeneous renal cell population is identified as having a therapeutic potential if it is determined that at least about 85% of cells of the heterogeneous renal cell population express RACK-1.

37. The method of claim 30, wherein the enriched heterogeneous renal cell population is identified as having a therapeutic potential if it is determined that about 4% to about 99% of cells of the heterogeneous renal cell population express nephrin and at least about 85% of cells of the heterogeneous renal cell population express RACK-1.

38. The method of claim 31, wherein the enriched heterogeneous renal cell population is identified as having a therapeutic potential if it is determined that at least about 90% of cells of the heterogeneous renal cell population express podocin and at least about 85% of cells of the heterogeneous renal cell population express RACK-1.

39. The method of claim 32, wherein the enriched heterogeneous renal cell population is identified as having a therapeutic potential if it is determined that about 4% to about 99% of cells of the heterogeneous renal cell population express nephrin, at least about 90% of cells of the heterogeneous renal cell population express podocin and at least about 85% of cells of the heterogeneous renal cell population express RACK-1.

40. The method of any of claims 1-39, further comprising:
determining whether cells of the enriched heterogeneous renal cell population express one or more of BMP4, BMP7, GDNF, HOX11, EYA1, SAL1 and SIX4; and
identifying the enriched heterogeneous renal cell population as having therapeutic potential if it is determined that cells of the enriched heterogeneous renal cell population express one or more of BMP4, BMP7, GDNF, HOX11, EYA1, SAL1 and SIX4.

41. The method of any if claims 1-40, further comprising:
determining whether cells of the enriched heterogeneous renal cell population express one or more of PAX2, CITED1, FGFR1, FGF7, FGF10, HOX10, POD1 and MUC1; and
identifying the enriched heterogeneous renal cell population as having therapeutic potential if it is determined that cells of the enriched heterogeneous renal cell population express one or more of PAX2, CITED1, FGFR1, FGF7, FGF10, HOX10, POD1 and MUC1.

42. The method of claim 41, wherein the one or more comprises PAX2.

43. The method of any of claims 1-42, further comprising:
determining whether cells of the enriched heterogeneous renal cell population express one or more of BMP7, SMAD, LIF, FOXD1, HOXB7, ALK3, DKK1 and SPRY1; and
identifying the enriched heterogeneous renal cell population as having therapeutic potential if it is determined that cells of the enriched heterogeneous renal cell population express one or more of BMP7, SMAD, LIF, FOXD1, HOXB7, ALK3, DKK1 and SPRY1.

44. The method of any one of claims 1-43, further comprising:
determining whether cells of the enriched heterogeneous renal cell population express one or more of genes Receptor for Hyaluronan Mediated Motility (RHAMM), C2, C3, C4, fibrinogen, coagulation factor XIII, TEK, KDR, Notch1, Notch3, Timp3, Vwf, Adam15, Gas6, Igfbp1, or Tm4sf4; and
identifying the enriched heterogeneous renal cell population as having therapeutic potential if it is determined that cells of the enriched heterogeneous renal cell population express the one or more of genes RHAMM, C2, C3, C4, fibrinogen, coagulation factor XIII, TEK, KDR, Notch1, Notch3, Timp3, Vwf, Adam15, Gas6, Igfbp1, or Tm4sf4.

45. The method of claim 44, wherein the one or more genes comprises RHAMM.

46. An enriched heterogeneous renal cell population identified according to the method of any of claims 1-45.

47. A pharmaceutical composition comprising the enriched heterogeneous renal cell population of claim 46.

48. A method of treating kidney disease in a patient in need thereof, the method comprising:
administering a therapeutically effective amount of the pharmaceutical composition of claim 47.

49. Use of the pharmaceutical composition of claim 47 in the manufacture of a medicament to treat kidney disease.

50. A method of identifying an enriched heterogeneous renal cell population as having a therapeutic potential, comprising:
determining expression level of one or more of genes RHAMM, C2, C3, C4, fibrinogen, coagulation factor XIII, TEK, KDR, Notch1, Notch3, Timp3, Vwf, Adam15, Gas6, Igfbp1, and Tm4sf4 by cells of the enriched heterogeneous renal cell population; and
identifying the enriched heterogeneous renal cell population as having therapeutic potential if the expression level of the one or more genes by cells of the enriched heterogeneous renal cell population is increased relative to expression level of the one or more genes by cells of a control renal cell population.

51. The method of claim 50, wherein the one or more genes comprises RHAMM.

52. The method of claim 50 or 51, wherein the enriched heterogeneous renal cell population is prepared via a method comprising a step of density gradient separation and the control renal cell population comprises cells subjected to the step of density gradient separation.

53. The method of claim 52, wherein the enriched heterogeneous renal cell population, prepared via the method, comprises cells of a buoyant density greater than about 1.04 g/mL.

54. An enriched heterogeneous renal cell population identified according to the method of any of claims 50-53.

55. A pharmaceutical composition comprising the enriched heterogeneous renal cell population of claim 54.

56. The pharmaceutical composition of claim 55 further comprising hyaluronic acid.

57. A method of treating kidney disease in a patient in need thereof, the method comprising:
administering a therapeutically effective amount of the pharmaceutical composition of claim 55 or 56.

58. Use of the pharmaceutical composition of claim 55 or 56 in the manufacture of a medicament to treat kidney disease.

59. A method of identifying an enriched heterogeneous renal cell population as having a therapeutic potential, comprising:
determining whether cells of the enriched heterogeneous renal cell population express SIX2, OSR1, RET and podocin; and
identifying the enriched heterogeneous renal cell population as having therapeutic potential if cells of the enriched heterogeneous renal cell population are determined to express SIX2, OSR1, RET and podocin.

60. The method of claim 59 wherein the enriched heterogeneous renal cell population is identified as having a therapeutic potential if it is determined that greater than 0% and at most about 10% of cells of the enriched heterogeneous renal cell population express SIX2.

61. The method of claim 60, wherein the enriched heterogeneous renal cell population is identified as having a therapeutic potential if it is determined that greater than 0% and at most about 6% of cells of the enriched heterogeneous renal cell population express SIX2.

62. The method of any of claims 59-61, wherein the enriched heterogeneous renal cell population is identified as having a therapeutic potential if it is determined that at least about 36% of cells of the enriched heterogeneous renal cell population express OSR1.

63. The method of any of claims 59-62, wherein the enriched heterogeneous renal cell population is identified as having a therapeutic potential if it is determined that about 36% to about 85% of cells of the enriched heterogeneous renal cell population express OSR1.

64. The method of any of claims 59-63, wherein the enriched heterogeneous renal cell population is identified as having a therapeutic potential if it is determined that greater than 0% and up to at most about 90% of cells of the enriched heterogeneous renal cell population express RET.

65. The method of any of claims 59-64, wherein the enriched heterogeneous renal cell population is identified as having a therapeutic potential if it is determined that greater than 85% of cells of the population express podocin.

66. The method of any of claims 59-65, further comprising:
determining whether cells of the enriched heterogeneous renal cell population express one or more of LHX1, FGF8, RACK-1 or nephron; and
identifying the enriched heterogeneous renal cell population as having therapeutic potential if cells of the enriched heterogeneous renal cell population are determined to express one or more of LHX1, FGF8, RACK-1 or nephron.

67. The method of claim 66, wherein the one or more comprises LHX1.

68. The method of claim 67, wherein the enriched heterogeneous renal cell population is identified as having a therapeutic potential if it is determined that about 8% to about 58% of cells of the enriched heterogeneous renal cell population are determined to express LHX1

69. The method of any of claims 66-68, wherein the one or more comprises FGF8.

70. The method of claim 69, wherein the enriched heterogeneous renal cell population is identified as having a therapeutic potential if it is determined that about 0.48% to about 59% of cells of the enriched heterogeneous renal cell population express FGF8.

71. The method of any of claims 66-70, wherein the one or more comprises RACK-1.

72. The method of claim 71, wherein the enriched heterogeneous renal cell population is identified as having a therapeutic potential if it is determined that at least about 85% of cells of the heterogeneous renal cell population express RACK-1.

73. The method of any of claims 66-72, wherein the one or more comprises nephrin.

74. The method of claim 73, wherein the enriched heterogeneous renal cell population is identified as having a therapeutic potential if it is determined that about 4% to about 99% of cells of the heterogeneous renal cell population express nephrin.

75. A method of identifying an enriched heterogeneous renal cell population as having a therapeutic potential, comprising:
determining whether cells of the enriched heterogeneous renal cell population express nephrin, podocin and LHX1; and
identifying the enriched heterogeneous renal cell population as having therapeutic potential if cells of the population express nephrin, podocin and LHX1.

76. The method of claim 75, wherein the enriched heterogeneous renal cell population is identified as having a therapeutic potential if it is determined that greater than 70% of cells of the enriched heterogeneous renal cell population express nephrin.

77. The method of claim 76, wherein the enriched heterogeneous renal cell population is identified as having a therapeutic potential if it is determined that greater than 75% of cells of the enriched heterogeneous renal cell population express nephrin.

78. The method of any of claims 75-77, wherein the enriched heterogeneous renal cell population is identified as having a therapeutic potential if it is determined that greater than 80% of cells of the enriched heterogeneous renal cell population express podocin.

79. The method of claim 78, wherein the enriched heterogeneous renal cell population is identified as having a therapeutic potential if it is determined that greater than 90% of cells of the enriched heterogeneous renal cell population express podocin.

80. The method of any of claims 75-79, wherein the enriched heterogeneous renal cell population is identified as having a therapeutic potential if it is determined that greater than 10% of cells of the enriched heterogeneous renal cell population express LHX1.

81. The method of claim 80, wherein the enriched heterogeneous renal cell population is identified as having a therapeutic potential if it is determined that greater than 20% of cells of the enriched heterogeneous renal cell population express LHX1.

82. The method of any of claims 75-81, further comprising
determining whether cells of the enriched heterogeneous renal cell population express one or more of SIX2, OSR1, RET, FGF8 or RACK-1; and
identifying the enriched heterogeneous renal cell population as having therapeutic potential if cells of the enriched heterogeneous renal cell population are determined to express one or more of SIX2, OSR1, RET, FGF8 or RACK-1.

83. The method of claim 82, wherein the one or more comprises SIX2.

84. The method of claim 83, wherein the enriched heterogeneous renal cell population is identified as having a therapeutic potential if it is determined that greater than 0% and at most about 15% of cells of the enriched heterogeneous renal cell population express SIX2.

85. The method of any of claims 82-84, wherein the one or more comprises OSR1.

86. The method of claim 85, wherein the enriched heterogeneous renal cell population is identified as having a therapeutic potential if it is determined that about 36% to about 84% of cells of the enriched heterogeneous renal cell population express OSR1.

87. The method of any of claims 82-86, wherein the one or more comprises RET.

88. The method of claim 87, wherein the enriched heterogeneous renal cell population is identified as having a therapeutic potential if it is determined that greater than 0% and up to at most about 90% of cells of the enriched heterogeneous renal cell population express RET.

89. The method of any of claims 82-88, wherein the one or more comprises FGF8.

90. The method of claim 89, wherein the enriched heterogeneous renal cell population is identified as having a therapeutic potential if it is determined that about 0.48% to about 59% of cells of the enriched heterogeneous renal cell population express FGF8.

91. The method of any of claims 82-90, wherein the one or more comprises RACK-1.

92. The method of claim 91, wherein the enriched heterogeneous renal cell population is identified as having a therapeutic potential if it is determined that at least about 85% of cells of the heterogeneous renal cell population express RACK-1.

93. A pharmaceutical composition comprising the enriched heterogeneous renal cell population identified as having therapeutic potential according to the method of any of claims 75-92.

94. A method of treating kidney disease in a patient in need thereof, the method comprising:
administering a therapeutically effective amount of the pharmaceutical composition of claim 93.

95. Use of the pharmaceutical composition of claim 93 in the manufacture of a medicament to treat kidney disease.
